(19) United States
(12) Patent Application Publication
(10) Pub. No.: US 2011/0313672 A1
(43) Pub. Date: Dec. 22, 2011

(54) CELLULAR ANTIOXIDANT ACTIVITY (CAA) ASSAY

(75) Inventors: Rui Hai Liu, Ithaca, NY (US); Kelly L. Nehmer, Decatur, IL (US)

(73) Assignee: CORNELL UNIVERSITY, Ithaca, NY (US)

(21) Appl. No.: 12/674,544

(22) PCT Filed: Sep. 11, 2008
(86) PCT No.: PCT/US2008/075947

§ 371 Date: Jun. 29, 2011

Related U.S. Application Data
(60) Provisional application No. 60/993,246, filed on Sep. 11, 2007.

(51) Int. Cl. G06F 19/00; C12Q 1/02

(52) U.S. Cl. 702/19; 435/29

(57) ABSTRACT

A cellular antioxidant activity (CAA) assay for quantifying the antioxidant activity of phytochemicals, food extracts, and dietary supplements has been developed. Dichlorofluorescin is a probe that is trapped within cells and is easily oxidized to fluorescent dichlorofluorescein (DCF). The method measures the ability of compounds to prevent the formation of DCF by 2,2′-azo-bis(2-amidinopropane) dihydrochloride (ABAP)-generated peroxyl radicals in human hepatocarcinoma HepG2 cells. The decrease in cellular fluorescence when compared to the control cells indicates the antioxidant capacity of the compounds. The antioxidant activities of selected phytochemicals and fruit extracts were evaluated using the CAA assay and the results were expressed in μ-mol quercetin equivalents/100 μ-mol phytochemical or μ-mol quercetin equivalents/100 g fresh fruit. Quercetin had the highest CAA value, followed by kaempferol, epigallocatechin gallate (EGCG), myricetin, and luteolin among the pure compounds tested. Among the selected fruits tested, blueberry had the highest CAA value, followed by cranberry>apple=red grape>green grape. The CAA assay is a more biologically relevant method than the popular chemistry antioxidant activity assays because it accounts for aspects of uptake, metabolism, and location of species within cells.

### FIELD OF THE INVENTION

The present invention relates to methods for measuring and standardizing antioxidant capacity of a plant extract or test compound.

### BACKGROUND OF THE INVENTION

Heart disease and cancer are the two leading causes of death in the United States (Minino, A. et al (2006) Deaths: Preliminary Data for 2004.; National Center for Health Statistics: Hyattsville, Md.) and oxidative stress is thought to be an important contributing factor in their development. Oxidative stress is an imbalance between the production of reactive oxygen species (ROS) and antioxidant defense and may lead to oxidative damage (Ames, B. N.; and Gold, L. S., (1991) Mutat. Res. 250(1-2):3-16; Halliwell, B. and Gutteridge, J. M. C., (1999) Free Radicals in Biology and Medicine. 3rd ed.; Oxford University Press, Inc.: New York). It can result from a deficiency in antioxidant defense mechanisms, or from an increase in ROS, due to exposure to elevated ROS levels, the presence of toxins metabolized to ROS, or excessive activation of ROS systems, such as those mediated by chronic infection and inflammation (Liu, R. H. and Hotchkiss, J. H., (1995) Mutat. Res. 339(2):73-89). In addition to endogenously produced antioxidants and enzymes that catalyze the metabolism of ROS, ROS can be scavenged by exogenously obtained antioxidants, such as phenolics, carotenoids, and vitamins found in fruits and vegetables. Fruits and vegetables are excellent sources of phenolic compounds (Chu, et al (2002) J. Agric. Food Chem. 50(23):6910-6; Sun, J. et al (2002) J. Agric. Food Chem. 50 (25):7449-54). Consumption of these compounds from dietary plant sources may increase protective antioxidants in the body and help combat cardiovascular diseases and cancer, as supported by epidemiological studies (Block, G. et al (1992) Nutr. Cancer. 18(1):1-29; Bazzano, L. A. et al (2002) Am. J. Clin. Nutr. 76(1):93-9; Hung, H. C. et al (2004) J. Natl. Cancer Inst. 96 (21):1577-84; Joshipura, K. J. (2001) Ann. Intern. Med. 134(12):1106-14; Liu, S. et al (2000) Am. J. Clin. Nutr. 72(4):922-8; Smith-Warner, S. A, et al (2003) Int. J. Cancer 107(6):1001-11; Steinmetz, K. A and Potter, J. D., (1006). J. Am. Diet Assoc. 96(10):1027-39). The 2005 Dietary Guidelines for Americans recommends consumption of at least 4 servings of fruits and 5 servings of vegetables per day based on a dietary requirement of 2000 kcalories (U.S. Department of Health and Human Services and U.S. Department of Agriculture., Dietary Guidelines for Americans, 2005. 6 ed.; Government Printing Office Washington, D.C., 2005).

The measurement of antioxidant activity is an important screening method to compare the oxidation/reduction potentials of fruits and vegetables and their phytochemicals in various systems. Many chemistry methods are currently in wide use, including the Oxygen-Radical Absorbance Capacity (ORAC)(Cao, G.; et al, (1993) Free Radic. Biol. Med. 14(3):303-11), Total Radical-Trapping Antioxidant Parameter (TRAP) (Ghiselli, A.; et al (1995) Free Radic. Biol. Med. 18(1):29-36; Wayner, D. D et al (1985) FEBS Lett. 187(1):33-7), Trolox Equivalent Antioxidant Capacity (TEAC) (Miller, N. J.; et al (1993) Clin. Sci. (Lond) 84(4):407-12), Total Oxyradical Scavenging Capacity (TOSC) (Winston, G. W.; et al (1998) Free Radic. Biol. Med. 24(3):480-93), and the Peroxyl Radical Scavenging Capacity (PSC) assay recently developed by our laboratory (Adom, K. K.; et al (2005). J. Agric. Food Chem. 53(17):6572-80), all of which determine the ability of substances to delay or quench ROS produced by free radical generators. The Ferric Reducing/Antioxidant Power (FRAP) assay (Benzie, I. F.; et al (1996) Anal. Biochem. 239(1):70-6) and the DPPH free radical method (Brand-Williams, W. et al (1995) Lebensm. Wiss. Technol. 28(1):25-30) measure the ability of antioxidants to reduce ferric iron and 2,2-diphenyl-picrylhydrazyl, respectively.

Despite wide usage of these chemical antioxidant activity assays, their ability to predict in vivo activity is questioned for a number of reasons. Some are performed at non-physiological pH and temperature, and none of them take into account the bioavailability, uptake, and metabolism of the antioxidant compounds (Liu, R. H. and Finley, J., (2005) J. Agric. Food Chem 53(10):4311-4). The protocols often do not include the appropriate biological substrates to be protected, relevant types of oxidants encountered, or the partitioning of compounds between the water and lipid phases and the influence of interfacial behavior (Frankel, E. N and Meyer, A. S., (2000) J. Sci. Food. Agric. 80(13):1925-1941).

Biological systems are much more complex than the simple chemical mixtures employed and antioxidant compounds may operate via multiple mechanisms (Liu, R. H., (2004) J. Nutr. 134(12):34795-3485). The different efficacies of compounds in the various assays attest to the functional variation. The best measures are from animal models and human studies; however, these are expensive and time-consuming and not suitable for initial antioxidant screening of foods and dietary supplements (Liu, R. H. and Finley, J., (2005), supra). Cell culture models provide an approach that is cost-effective, relatively fast, and addresses some issues of uptake, distribution, and metabolism.

### SUMMARY OF THE INVENTION

Described herein is a cell-based antioxidant activity assay to screen foods, phytochemicals and dietary supplements for potential biological activity by determining the antioxidant capacity. Also described herein is a method for determining a standardized antioxidant capacity for a plant extract, plant mixture, or purified compound that can be used to compare values among laboratories, to compare values measured at different times or by different users, or to compare the antioxidant capacity of unrelated compounds or extracts.

One aspect described herein is a method of measuring antioxidant capacity of a test compound, the method comprising the steps of: (a) contacting a cultured cell with 2′,7′-dichlorofluorescin diacetate in the presence and absence of a test compound, wherein the 2′,7′-dichlorofluorescin diacetate enters the cell and is cleaved to 2′,7′-dichlorofluorescin; (b) contacting the cell with a peroxyl radical initiator; (c) measuring fluorescence in an emission wavelength of 2′,7′-dichlorofluorescein at a plurality of time points, (d) determining the area-under-the-curve of a graph plotting 2′,7′-dichlorofluorescein diacetate fluorescence vs. time for the cultured cell in the presence and absence of the test compound, wherein a decrease in the area-under-the-curve in the presence of the test compound, relative to the area-under-the-curve in the absence of the test compound indicates antioxidant capacity of the test compound.

Another aspect disclosed herein is a method of predicting in vivo antioxidant capacity of a compound, the method comprising the steps of: (a) contacting a first cultured cell with 2′,7′-dichlorofluorescin diacetate, in the presence of a test compound to form a first mixture, (b) contacting a second cultured cell with 2′,7′-dichlorofluorescin diacetate, in the absence of the test compound to form a second mixture, wherein the 2′,7′-dichlorofluorescin diacetate enters the first and the second cells and is cleaved therein to 2′,7′-dichlorofluorescin; (c) contacting the first and second mixtures with a peroxyl radical initiator; (d) measuring fluorescence in an emission wavelength of 2′,7′-dichlorofluorescein at a plurality of time points in the first and the second mixtures, and (e) determining area-under-the-curve of a graph plotting 2′,7′-dichlorofluorescein diacetate fluorescence vs. time for the first and second mixtures, wherein a decrease in the area-under-the-curve at an emission wavelength of 2′,7′-dichlorofluorescein in the first mixture, relative to the area-under-the-curve in the second mixture provides a prediction of in vivo antioxidant capacity of the test compound.

Another aspect disclosed herein is a kit for measuring the antioxidant capacity of a compound, the kit comprising: (a) 2′,7′-dichlorofluorescin diacetate; (b) a peroxyl radical initiator; (c) a standard; (d) a computer readable medium comprising instructions for determining antioxidant capacity of a test compound, and (e) packaging materials therefor.

Another aspect disclosed herein is a method for determining an absolute value of antioxidant activity for a test compound, the method comprising the steps of: (a) contacting a first cultured cell with 2′,7′-dichlorofluorescin diacetate, in the presence of a test compound, (b) contacting a second cultured cell with 2′,7′-dichlorofluorescin diacetate, in the absence of the test compound, wherein the 2′,7′-dichlorofluorescin diacetate enters the first and second cells and is cleaved therein to 2′,7′-dichlorofluorescin; (c) contacting the first and second cells with a peroxyl radical initiator; and (d) measuring fluorescence in an emission wavelength of 2′,7′-dichlorofluorescein at a plurality of time points in the first and second cultured cells, (e) determining the ratio of area-under-the-curve of a graph plotting 2′,7′-dichlorofluorescein diacetate fluorescence vs. time for the first and second cultured cells, (f) normalizing the ratio of area-under-the-curve of step (e) to area-under-the-curve of a graph plotting 2′,7′-dichlorofluorescein diacetate fluorescence vs. time for a standard compound.

Another aspect described herein is a computer-readable medium comprising instructions for obtaining an absolute antioxidant value from fluorescence measured at a plurality of time points, the medium comprising: (a) instructions for receiving a plurality of fluorescence values, the values representing fluorescence at a plurality of time points for a cultured cell in the presence and absence of a test compound; (b) instructions for receiving a plurality of fluorescence values, the values representing fluorescence at a plurality of time points for a cultured cell in the presence of a standard compound; (c) instructions for calculating an absolute antioxidant value, CAA_{abs}, for the test compound, the instructions comprising applying the values received according to instructions (a) and (b) to the relationship of Equation (1)

$$CAA abs = \frac{( 1 - ( \int SA / \int CA ) )}{( 1 - ( \int Sq / \int CA ) )} \qquad Equation ( 1 )$$

wherein ∫SA is the area-under-the-curve for fluorescence vs. time of the test compound, ∫CA is the area-under-the-curve for fluorescence vs. time in the absence of the test compound, and ∫S_{q} is the area-under-the-curve for fluorescence vs. time of the standard compound; and (d) instructions for transmitting a value for CAA_{abs} to an output device.

### BRIEF DESCRIPTION OF THE DRAWINGS

FIG. 1. Total phenolic contents of selected fruits (mean±SD, n=3). Bars with different letters are significantly different (p<0.05).

FIG. 2. Method and proposed principle of the cellular antioxidant activity (CAA) assay. Cells were pretreated with antioxidant compounds or fruit extracts and DCFH-DA. The antioxidants bound to the cell membrane and/or passed through the membrane to enter the cell. DCFH-DA diffused into the cell where cellular esterases cleaved the diacetate moiety to form the more polar DCFH, which was trapped within the cell. Cells were treated with ABAP, which was able to diffuse into cells. ABAP spontaneously decomposed to form peroxyl radicals. These peroxyl radicals attacked the cell membrane to produce more radicals and oxidized the intracellular DCFH to the fluorescent DCF. Antioxidants prevented oxidation of DCFH and membrane lipids and reduced the formation of DCF.

FIG. 3. Peroxyl radical-induced oxidation of DCFH to DCF in HepG2 cells, and the inhibition of oxidation by quercetin (A, B), gallic acid (C, D), and blueberry extracts (E, F) over time, using the protocol involving no PBS wash between antioxidant and ABAP treatments (A, C, E) and the protocol with a PBS wash (B, D, F), to remove antioxidants in the medium not associated with cells. The curves shown in each graph are from a single experiment (mean±SD, n=3).

FIG. 4. Dose-response curves for inhibition of peroxyl radical-induced DCFH oxidation by quercetin (A, B) and blueberry extracts (C, D) without a PBS wash between treatments in the protocol involving no PBS wash between antioxidant and ABAP treatments (A, C) and the protocol with a PBS wash (B, D). The curves shown are each from a single experiment (mean±SD, n=3).

FIG. 5. Median effect plots for inhibition of peroxyl radical-induced DCFH oxidation by quercetin (A, B) and blueberry extracts (C, D) in the protocol involving no PBS wash between antioxidant and ABAP treatments (A, C) and the protocol with a PBS wash (B, D). The curves shown are from a single experiment (n=3).

FIG. 6. Cellular antioxidant activity (CAA) of selected pure phytochemical compounds (mean±SD, n=3). Bars with different letters are significantly different (p<0.05).

FIG. 7. Cellular antioxidant activity (CAA) of selected fruits (mean±SD, n=3). Bars with different letters are significantly different (p<0.05).

FIG. 8. Total phenolic content of selected fruits (mean±SD, n=3). Bars with no letters in common are significantly different (p<0.05).

FIG. 9. ORAC values of selected fruits (mean±SD, n=3). Bars with no letters in common are significantly different (p<0.05).

FIG. 10. CAA values of selected fruits in the no PBS wash protocol (mean±SD, n=3). Bars with no letters in common are significantly different (p<0.05).

FIG. 11. CAA values of selected fruits with quantifiable activity in the PBS wash protocol (mean±SD, n=3). Bars with no letters in common are significantly different (p<0.05).

FIG. 12. Contribution of total phenolics from selected fruits as a percent of total phenolics from all fruits consumed by Americans.

FIG. 13. Contribution of (A) CAA from no PBS wash protocol and (B) CAA from PBS wash protocol from selected fruits as a percent of total cellular antioxidant activity from all fruits consumed by Americans.

FIG. 14. Generic structure of flavonoids.

FIG. 15. EC₅₀ values for selected flavonoids in the CAA assay (mean±SD, n=3). In each graph, bars having no letters in common are significantly different (p<0.05).

FIG. 16. Structures of flavonoids showing differences in B-ring hydroxylation within subclasses.

FIG. 17. Flavonoids with similar B-ring hydroxylation patterns and different C-ring structural features.

FIG. 18. Quercetin glycoside structures.

FIG. 19. Isoflavone structures.

FIG. 20. Structures of flavanols (catechins).

### DETAILED DESCRIPTION

Described herein is a quantifiable cellular antioxidant activity (CAA) assay, which is an improvement over the currently used “test tube” chemistry methods of measuring antioxidant activity (e.g., ORAC assay). This model better represents the complexity of biological systems than the popular chemistry antioxidant activity assays and is an important tool for screening foods, phytochemicals, and dietary supplements for potential biological activity.

One aspect described herein is a method of measuring antioxidant capacity of a test compound, the method comprising the steps of: (a) contacting a cultured cell with 2′,7′-dichlorofluorescin diacetate in the presence and absence of a test compound, wherein the 2′,7′-dichlorofluorescin diacetate enters the cell and is cleaved to 2′,7′-dichlorofluorescin; (b) contacting the cell with a peroxyl radical initiator; (c) measuring fluorescence in an emission wavelength of 2′,7′-dichlorofluorescein at a plurality of time points, (d) determining the area-under-the-curve of a graph plotting 2′,7′-dichlorofluorescein diacetate fluorescence vs. time for the cultured cell in the presence and absence of the test compound, wherein a decrease in the area-under-the-curve in the presence of the test compound, relative to the area-under-the-curve in the absence of the test compound indicates antioxidant capacity of the test compound.

In one embodiment of this aspect and all other aspects described herein, the peroxyl radical initiator comprises a 2,2′-azobis(2-amidinopropane) salt.

In another embodiment of this aspect and all other aspects described herein, the 2,2′-azobis(2-amidinopropane) salt comprises 2,2′-azobis(2-amidinopropane) dihydrochloride.

In another embodiment of this aspect and all other aspects described herein, the method further comprises comparing the antioxidant capacity of the test compound to an antioxidant capacity of a standard compound, wherein the antioxidant capacity of a standard compound is generated by the steps of: (a) contacting a cultured cell with 2′,7′-dichlorofluorescin diacetate in the presence of a standard compound, wherein the 2′,7′-dichlorofluorescin diacetate enters the cell and is cleaved to 2′,7′-dichlorofluorescin; (b) contacting the cell with a peroxyl radical initiator; and (c) measuring fluorescence in an emission wavelength of 2′,7′-dichlorofluorescein at a plurality of time points, (d) determining the area-under-the-curve of a graph plotting 2′,7′-dichlorofluorescein diacetate fluorescence vs. time for the cultured cell in the presence the standard compound.

In another embodiment of this aspect and all other aspects described herein, the standard compound is selected from the group consisting of quercetin, galangin, EGCG and kaempferol.

In another embodiment of this aspect and all other aspects described herein, the emission wavelength is 538 nm.

In another embodiment of this aspect and all other aspects described herein, the test compound is produced by a plant.

In another embodiment of this aspect and all other aspects described herein, the test compound is a phytochemical.

In another embodiment of this aspect and all other aspects described herein, the cultured cell is a eukaryotic cell.

In another embodiment of this aspect and all other aspects described herein, the eukaryotic cell is a human cell.

In another embodiment of this aspect and all other aspects described herein, the eukaryotic cell is a cell of a human cell line.

In another embodiment of this aspect and all other aspects described herein, the human cell line is HepG2.

In another embodiment of this aspect and all other aspects described herein, the method further comprises a step of washing the cultured cell prior to the step of contacting the cell with the peroxyl initiator and comparing antioxidant activity data derived from washed cells with antioxidant activity data derived from unwashed cells.

Another aspect disclosed herein is a method of predicting in vivo antioxidant capacity of a compound, the method comprising the steps of: (a) contacting a first cultured cell with 2′,7′-dichlorofluorescin diacetate, in the presence of a test compound to form a first mixture, (b) contacting a second cultured cell with 2′,7′-dichlorofluorescin diacetate, in the absence of the test compound to form a second mixture, wherein the 2′,7′-dichlorofluorescin diacetate enters the first and the second cells and is cleaved therein to 2′,7′-dichlorofluorescin; (c) contacting the first and second mixtures with a peroxyl radical initiator; (d) measuring fluorescence in an emission wavelength of 2′,7′-dichlorofluorescein at a plurality of time points in the first and the second mixtures, and (e) determining area-under-the-curve of a graph plotting 2′,7′-dichlorofluorescein diacetate fluorescence vs. time for the first and second mixtures, wherein a decrease in the area-under-the-curve at an emission wavelength of 2′,7′-dichlorofluorescein in the first mixture, relative to the area-under-the-curve in the second mixture provides a prediction of in vivo antioxidant capacity of the test compound.

Another aspect disclosed herein is a kit for measuring the antioxidant capacity of a compound, the kit comprising: (a) 2′,7′-dichlorofluorescin diacetate; (b) a peroxyl radical initiator; (c) a standard; (d) a computer readable medium comprising instructions for determining antioxidant capacity of a test compound, and (e) packaging materials therefor.

Another aspect disclosed herein is a method for determining an absolute value of antioxidant activity for a test compound, the method comprising the steps of: (a) contacting a first cultured cell with 2′,7′-dichlorofluorescin diacetate, in the presence of a test compound, (b) contacting a second cultured cell with 2′,7′-dichlorofluorescin diacetate, in the absence of the test compound, wherein the 2′,7′-dichlorofluorescin diacetate enters the first and second cells and is cleaved therein to 2′,7′-dichlorofluorescin; (c) contacting the first and second cells with a peroxyl radical initiator; and (d) measuring fluorescence in an emission wavelength of 2′,7′-dichlorofluorescein at a plurality of time points in the first and second cultured cells, (e) determining the ratio of area-under-the-curve of a graph plotting 2′,7′-dichlorofluorescein diacetate fluorescence vs. time for the first and second cultured cells, (f) normalizing the ratio of area-under-the-curve of step (e) to area-under-the-curve of a graph plotting 2′,7′-dichlorofluorescein diacetate fluorescence vs. time for a standard compound.

In one embodiment of this aspect and all other aspects described herein, an absolute value of antioxidant activity is determined for a test compound by applying the fluorescent values obtained to Equation (1)

$$CAA abs = \frac{( 1 - ( \int SA / \int CA ) )}{( 1 - ( \int Sq / \int CA ) )} \qquad Equation ( 1 )$$

wherein ∫SA is the area-under-the-curve for fluorescence vs. time of the test compound, ∫CA is the area-under-the-curve for fluorescence vs. time in the absence of the test compound, and ∫S_{q} is the area-under-the-curve for fluorescence vs. time of the standard compound, and wherein CAA_{abs} is the absolute value of antioxidant activity for a test compound.

Another aspect described herein is a computer-readable medium comprising instructions for obtaining an absolute antioxidant value from fluorescence measured at a plurality of time points, the medium comprising: (a) instructions for receiving a plurality of fluorescence values, the values representing fluorescence at a plurality of time points for a cultured cell in the presence and absence of a test compound; (b) instructions for receiving a plurality of fluorescence values, the values representing fluorescence at a plurality of time points for a cultured cell in the presence of a standard compound; (c) instructions for calculating an absolute antioxidant value, CAA_{abs}, for the test compound, the instructions comprising applying the values received according to instructions (a) and (b) to the relationship of Equation (1)

$$CAA abs = \frac{( 1 - ( \int SA / \int CA ) )}{( 1 - ( \int Sq / \int CA ) )} \qquad Equation ( 1 )$$

wherein ∫SA is the area-under-the-curve for fluorescence vs. time of the test compound, ∫CA is the area-under-the-curve for fluorescence vs. time in the absence of the test compound, and ∫S_{q} is the area-under-the-curve for fluorescence vs. time of the standard compound; and (d) instructions for transmitting a value for CAA_{abs} to an output device.

As used above, and throughout the description of the present invention, the following terms, unless otherwise indicated, shall be understood to have the following meanings.

As used herein, the term “test compound” is used to describe a purified compound, an extract or a mixture derived from a plant and can be used for the purpose of measuring the antioxidant capacity of a fruit, green plant, or vegetable. In its simplest mode a plant is homogenized in an appropriate buffer and assayed using a whole plant mixture. If so desired, a portion of the mixture can be extracted using e.g., an organic phase separation method or a test compound can be purified by using e.g., affinity binding columns. These methods are well within the abilities of one skilled in the art to perform.

As used herein, the term “antioxidant capacity” is used to describe the ability of a test compound to produce an antioxidant effect in the presence of free radicals (i.e., quenching of oxidants). A test compound is considered to be an “antioxidant” if the compound is effective in reducing the amount of free radicals (as measured by 2′,7′-dichlorofluoroscein diacetate fluorescence) in a cultured cell by at least 10% compared to a cell not treated with the test compound; preferably the free radicals are reduced by at least 20%, at least 30%, at least 40%, at least 50%, at least 60%, at least 70%, at least 80%, at least 90%, at least 95%, at least 99%, or even 100% (i.e., absent) in cells cultured in the presence of the test compound compared to cells cultured in the absence of the compound. Since the methods described herein utilize cells for analysis, the antioxidant capacity encompasses both the membrane bound and the intracellular antioxidant capacity of a test compound. The “intracellular antioxidant capacity”, as that term is used herein includes both the bioavailability of the compound (i.e., the amount taken up by a cell), and the effect of the compound once it is internalized into the cell (i.e., the proportion of active compound remaining once intracellular metabolism or other alterations occur). Thus, the methods described herein are especially useful for predicting the antioxidant capacity of a test compound when administered to a subject in need thereof, referred to herein as “in vivo antioxidant capacity”. The antioxidant capacity of a compound is determined by plotting measured values for 2′,7′-dichlorofluorescein fluorescence vs. time in the presence and absence of the compound. The “area-under-the-curve”, as that term is used herein, refers to the integral of the function of fluorescence vs. time from t=0 (addition of peroxyl initiator) to t=final, wherein the final time point is determined by the beginning of the plateau phase of the fluorescent compound. It should be understood that where the term “plotting” is used, the term encompasses both literally plotting the data on a graph, as well as simply calculating the area-under-the-curve as the integral of the function of fluorescence vs. time from t=0 to t=final, i.e., without necessarily using a graph.

In one embodiment of the methods described herein, the “absolute antioxidant capacity” of a test compound is determined. The term “absolute antioxidant capacity” or “absolute value of antioxidant capacity” as used herein, refers to a standardized value for antioxidant capacity of a test compound that can be used to compare the antioxidant capacity among different test compounds, different assay times, different laboratories, different periods of time, and/or different users. The “absolute antioxidant capacity” refers to the antioxidant capacity of a test compound normalized to the antioxidant capacity of a standard compound.

A “standard compound” as that term is used herein, refers to a compound that has a high antioxidant capacity both in the presence and absence of a PBS wash as described herein. By “high antioxidant capacity” is meant at least 65 μmol of quercetin equivalents (QE)/100 μmol standard compound; preferably the standard compound has at least 70 μmol QE/100 μmol standard compound, at least 75, at least 80, at least 85, at least 90, at least 95, at least 99, at least 100 (i.e., quercetin), at least 200, at least 500, at least 1000, at least 10000, at least 100,000 or more μmol QE/100 μmol standard compound. In addition, an appropriate standard is one that is readily taken up into cells and thus has a high bioavailability. By “high bioavailability” is meant that the activity of the standard compound using a PBS wash is at least 50% of the activity of the standard compound in the absence of a PBS wash; preferably the activity is at least 60%, at least 70%, at least 80%, at least 90%, at least 95%, at least 99%, or even 100% (i.e., most or all of the compound is taken up into cells) in cells wherein a PBS wash is utilized compared to cells treated without the use of a PBS wash. In one embodiment, the standard compound is quercetin. In an alternative embodiment, the standard compound is ECGC or galangin. In another embodiment, the standard is kaempferol.

As used herein, the term “plurality of time points” means that fluorescence is measured at least 3 time points, at least 4, at least 5, at least 6, at least 7, at least 8, at least 9, at least 10, at least 15, at least 20, at least 30, at least 40, at least 50, at least 60, at least 70, at least 80, at least 90 time points or more. The total time necessary for an experiment will depend on the kinetics of the fluorescence/time curve and should be sufficiently long to permit adequate signaling but should not proceed past the plateau phase of the fluorescent compound in the absence of a test compound. Thus, to be clear the area-under-the-curve should be calculated during the linear phase of the curve from the time the peroxyl initiator is added until the fluorescence enters a plateau phase.

As used herein, the term “peroxyl radical initiator” refers to an oxidant compound that promotes the production of intracellular free radicals, thus shifting the balance of oxidants to antioxidants in favor of the oxidants. The peroxyl radical initiator may itself be a free radical, may be converted to a free radical, or in some cases may promote the production of a free radical from an intracellular source such as e.g., xanthine oxidase. In one embodiment, the peroxyl radical initiator is hydrogen peroxide. Alternatively, in another embodiment the peroxyl radical initiator is 2,2′-azobis(2-amidinopropane) dihydrochloride.

As used herein, the term “phytochemical” refers to a plant derived compound having, or having the potential for, health promoting properties.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

### Fruit Samples

Fruit samples can be obtained from any number of sources, including a local supermarket, a farmer's market, an orchard, a field etc. In the Examples described herein, wild blueberries were obtained from the Wild Blueberry Association of North America (Orono, Me.). Red Delicious apples were obtained from Cornell Orchards (Ithaca, N.Y.). Green and red seedless table grapes and frozen cranberries were purchased at a local supermarket (Ithaca, N.Y.).

### Fruit Extractions

Extracts are obtained from the fruits using e.g., an organic phase separation method such as described previously using eg., acetone (Sun, J. et al, (2002), supra), methanol, ethanol, ethyl acetate, and water. When organic solvents are desired for use, the solvents can be prepared as solutions comprising about 50-100% solvent, about 60-100%, about 70-100%, about 80-100%, about 90-100%, about 95-100%, about 99-100%, about 50-60%, about 50-70%, about 50-80%, about 50-90%, about 60-80%, about 65%-75% solvent or any range in between.

### Chemicals

Chemicals for the methods described herein can be obtained from a variety of commercial sources. For example, Folin-Ciocalteu reagent, 2′,7′-dichlorofluorescin diacetate (DCFH-DA), ethanol, glutaraldehyde, methylene blue, ascorbic acid, caffeic acid, (+)-catechin, (−)-epicatechin, (−)-epigallocatechin gallate (EGCG), ferulic acid, kaempferol, luteolin, myricetin, phloretin, quercetin dihydrate, resveratrol, and taxifolin are available for purchase from Sigma-Aldrich, Inc. (St. Louis, Mo.). Gallic acid can be obtained from ICN Biomedicals, Inc. (Aurora, Ohio). Dimethyl sulfoxide and acetic acid can be obtained from Fisher Scientific (Pittsburgh, Pa.) and 2,2′-azobis (2-amidinopropane) dihydrochloride (ABAP) are available for purchase from Wako Chemicals USA, Inc. (Richmond, Va.). Sodium carbonate, acetone, and methanol can be obtained from Mallinckrodt Baker, Inc. (Phillipsburg, N.J.). The HepG2 cells can be obtained from the American Type Culture Collection (ATCC) (Rockville, Md.). Williams' Medium E (WME) and Hanks' Balanced Salt Solution (HBSS) can be purchased from Gibco Life Technologies (Grand Island, N.Y.). Fetal bovine serum (FBS) can be obtained from Atlanta Biologicals (Lawrenceville, Ga.).

### Assay Medium

Essentially any cell culture medium can be used (e.g., WME, MEM, HBSS) with the exception of DMEM, which is known to increase the variability of the assay (data not shown). It is preferred that the variability among different sample or assay replicates is less than 30%, preferably less than 25%, less than 20%, less than 15%, less than 10%, less than 5%, less than 4%, less than 3%, less than 2%, less than 1%, less than 0.1%, less than 0.01% or more. Thus, a medium should be chosen that maintains variability among sample or assay replicates to a minimum (i.e., below 10% variability).

### Cells

Essentially any cell type can be used for the methods described herein but it is preferred that the cells utilized are eukaryotic or mammalian in origin. In this context, the cell can be of any cell type including, but not limited to, epithelial, endothelial, neuronal, adipose, cardiac, skeletal muscle, fibroblast, immune cells, hepatic, splenic, lung, circulating blood cells, reproductive cells, gastrointestinal, macrophage, lymphocyte, colon cells, renal, bone marrow, and pancreatic cells. The cell can be a cell line, a stem cell, or a primary cell isolated from any tissue including, but not limited to brain, liver, lung, gut, stomach, fat, muscle, testes, uterus, ovary, skin, endocrine organ and bone, etc. In one embodiment, the cell type is a human liver cell line, HepG2. When HepG2 cells are used, the cells are grown in growth medium (e.g., WME supplemented with 5% FBS, 10 mM Hepes, 2 mM L-glutamine, 5 μg/mL insulin, 0.05 μg/mL hydrocortisone, 50 units/mL penicillin, 50 μg/mL streptomycin, and 100 μg/mL gentamicin) and are maintained at 37° C. and 5% CO₂ as described previously (Liu, R. H.; et al (1994) Carcinogenesis 15(12):2875-7; Liu, R. H.; et a; (1992) Cancer Res. 52(15):4139-43). HepG2 cells should be used between passages 12 and 35.

### Preparation of Chemical and Fruit Sample Solutions

A 20 mM stock solution of DCFH-DA in methanol can be prepared, aliquoted, and stored at −20° C. A 200 mM ABAP stock solution is prepared and aliquots are stored at −40° C. Working phytochemical and fruit extract solutions should be prepared just prior to use. Caffeic acid, (+)-catechin, EGCG, (−)-epicatechin, ferulic acid, gallic acid, kaempferol, myricetin, phloretin, resveratrol, and taxifolin can be dissolved in ethanol, luteolin dissolved in methanol, and quercetin dissolved in dimethyl sulfoxide before further dilution in treatment medium (WME with 2 mM L-glutamine and 10 mM Hepes). Fruit extracts should be diluted in treatment medium. Final treatment solutions should contain less than 2% solvent to prevent cytotoxicity.

### Cytotoxicity

Cytotoxicity can be measured, for example by using the method of Oliver et al. (Oliver, M. H.; et al (1989) J. Cell Sci. 92(Pt 3):513-8) with slight modifications (Yoon, H.; et al (2007) J. Agric. Food Chem. 55(8):3167-3173). Briefly, HepG2 cells are seeded at 4×10⁴/well on a 96-well plate in 100 μL growth medium and incubated for 24 h at 37° C. The medium is removed and the cells are washed with PBS. Treatments of fruit extracts or antioxidant compounds in 100 μL treatment medium (Williams' Medium E supplemented with 2 mM L-glutamine and 10 mM Hepes) are applied to the cells and the plates are incubated at 37° C. for 24 h. The treatment medium is removed and the cells are washed with PBS. A volume of 50 μL/well methylene blue staining solution (98% HBSS, 0.67% glutaraldehyde, 0.6% methylene blue) is applied to each well and the plate is incubated at 37° C. for 1 h.

Excess dye is removed by immersing the plate in fresh deionized water until the water appears clear. The excess water should be tapped out of the wells and the plate allowed to air-dry briefly before addition of 100 μL elution solution (49% PBS, 50% ethanol, 1% acetic acid) to each well. The microplate is then placed on a bench-top shaker for 20 minutes to allow uniform elution. The absorbance is read at 570 nm with blank subtraction using, for example a MRX II DYNEX spectrophotometer (DYNEX Inc., Chantilly, Va.). Concentrations of pure compounds or fruit extracts that decrease the absorbance by more than 10% when compared to the control are considered to be cytotoxic.

### Exemplary Method for Performing the Cellular Antioxidant Activity of Pure Phytochemicals and Fruit Extracts

Cells (e.g., human hepatocellular carcinoma cells; HepG2) are seeded at a density of e.g., 6×10⁴/well on a 96-well microplate in 100 μL growth medium/well. It is preferred that only the inside wells of e.g., a 96-well microplate are used for the assay, since the outer wells have increased variation compared to that of the inner wells. Twenty-four hours after seeding the growth medium is removed and the wells are washed with PBS. Triplicate wells are treated for 1 h with 100 μL of a test compound (e.g., pure phytochemical compounds or fruit extracts) plus 25 μM DCFH-DA dissolved in treatment medium.

600 μM ABAP is then applied to the cells in 100 μL HBSS and the 96-well microplate is placed into a plate reader e.g., Fluoroskan Ascent FL plate-reader (ThermoLabsystems, Franklin, Mass.) at 37° C. Emission at 538 nm is measured with excitation at 485 nm, for example every 5 min for 1 h. Each plate should include triplicate control and blank wells: control wells contain cells treated with DCFH-DA and oxidant; blank wells contain cells treated with dye and HBSS without oxidant.

### PBS Wash

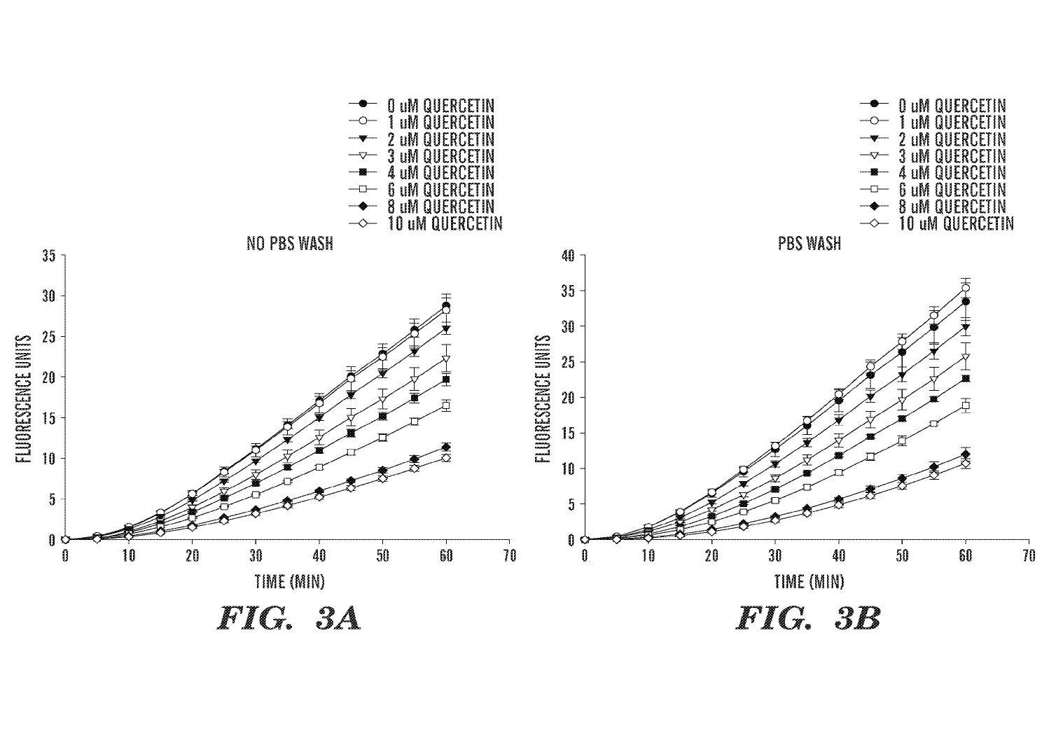

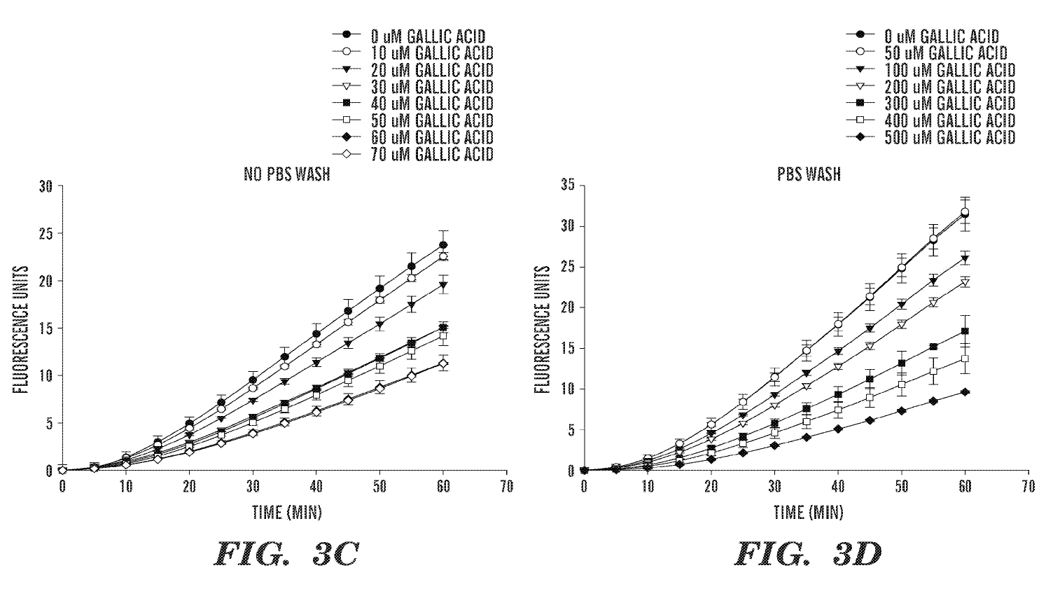

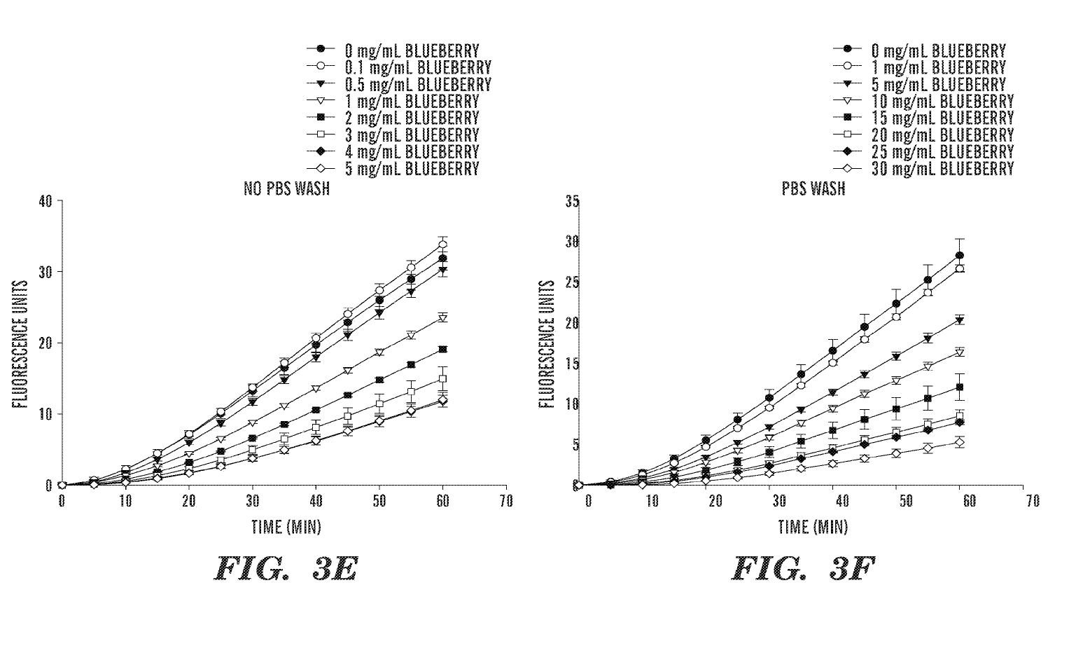

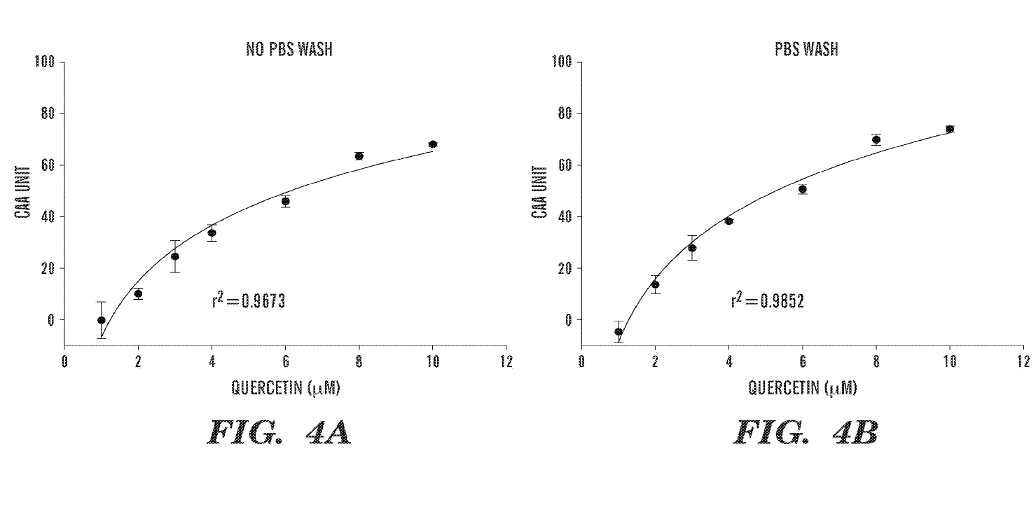

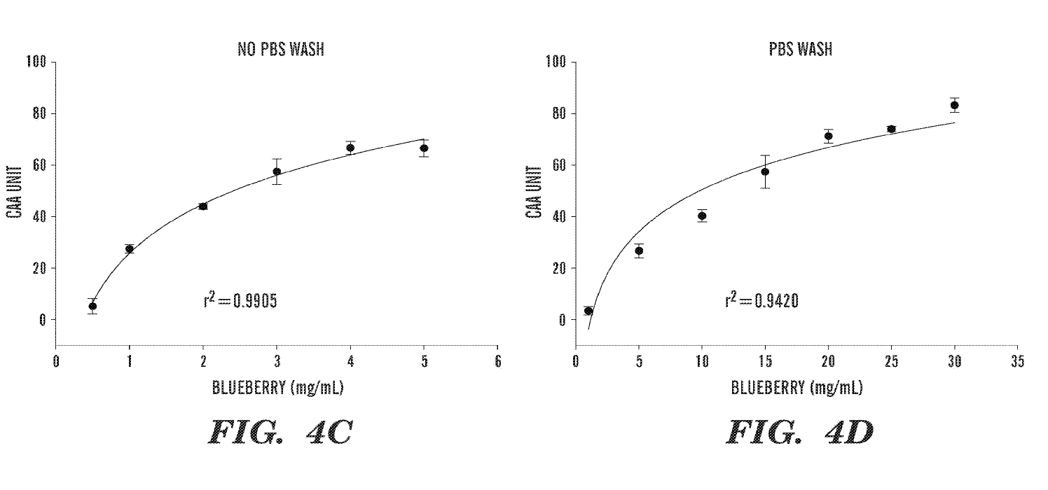

A test compound is assayed in multiple wells of e.g., a 96-well cell culture plate. Some wells are washed with 100 μL of phosphate-buffered saline (PBS) prior to the addition of ABAP, while other wells were not washed prior to the addition of ABAP. It was noted that the measured antioxidant activity of some fruit extracts (e.g., blueberries) was different when a PBS wash was used compared to when there was no PBS wash (see Table 2; FIGS. 3 and 4). This is likely due to non-specific binding of certain antioxidant compounds to the outer membrane, or a reduced uptake of the compound, thus the ratio of antioxidant activity values obtained with a PBS wash compared to a non-PBS wash indicates the bioavailability of the test compound. In order to accurately measure the intracellular antioxidant activity of a test compound, a PBS wash prior to the addition of a peroxyl initiator (e.g, ABAP) is necessary. The values obtained for cells assayed using a PBS wash should be compared to values obtained for the same cells assayed without a PBS wash. One of skill in the art can plan and perform experiments on a test compound in the presence and absence of a PBS wash, in order to accurately assess the intracellular antioxidant capacity of the test compound.

### Quantification of Cellular Antioxidant Activity (CAA).

After blank subtraction from the fluorescence readings, the area under the curve of fluorescence versus time is integrated to calculate the cellular antioxidant activity (CAA) value at each concentration of pure phytochemical compound or fruit extract as follows:

CAA unit=100−(∫SA/∫CA)×100

where ∫SA is the integrated area under the sample fluorescence versus time curve and ∫CA is the integrated area from the control curve. The median effective dose (EC₅₀) was determined for the pure phytochemical compounds and fruit extracts from the median effect plot of log (fₐ/fᵤ) vs. log (dose), where fₐ is the fraction affected and fᵤ is the fraction unaffected by the treatment. To quantify intra-experimental variation, the EC₅₀ values are stated as mean±SD for triplicate sets of data obtained from the same experiment. Inter-experimental variation is obtained for some representative pure phytochemical compounds and fruit extracts by averaging the fluorescence values from triplicate wells in each trial to obtain one EC₅₀ value per experiment and calculating the mean±SD for at least four trials. In each experiment, a standard is used, for example quecetin, thus permitting the cellular antioxidant activities for a test compounds to be expressed as μmol quercetin equivalents (QE)/100 μmol compound. Fruit extracts are expressed as μmol QE/100 g fruit. In order to compare the antioxidant quality of different fruits, cellular antioxidant activity (CAA) is also calculated as μmol QE/100 μmol total phenolics.

### Determination of Total Phenolic Content

The total phenolic contents of the fruit extracts can be determined using the Folin-Ciocalteu colorimetric method (Singleton, V. et al (1999), supra), as modified by the Liu laboratory (Dewanto, V. et al (2002), supra; Wolfe, K. et al (2003), supra). Results can be expressed as mean μmol gallic acid equivalents (GAE)/100 g fresh fruit±SD for three replicates.

### Statistical Analyses

Comparisons between two means can be performed using unpaired Student's t-tests. When there are more than two means, differences can be detected by ANOVA followed by multiple comparisons using Fisher's least significant difference test. Differences are considered to be significant when p<0.05.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents. The present invention may be as defined in any one of the following numbered paragraphs.
1. A method of measuring antioxidant capacity of a test compound, the method comprising the steps of:
   a) contacting a cultured cell with 2′,7′-dichlorofluorescin diacetate in the presence and absence of a test compound, wherein said 2′,7′-dichlorofluorescin diacetate enters said cell and is cleaved to 2′,7′-dichlorofluorescin;
   b) contacting said cell with a peroxyl radical initiator; and
   c) measuring fluorescence in an emission wavelength of 2′,7′-dichlorofluorescein at a plurality of time points,
   d) determining the area-under-the-curve of a graph plotting 2′,7′-dichlorofluorescein diacetate fluorescence vs. time for said cultured cell in the presence and absence of said test compound,
   wherein a decrease in said area-under-the-curve in the presence of said test compound, relative to said area-under-the-curve in the absence of said test compound indicates antioxidant capacity of said test compound.
2. The method of paragraph 1 wherein said peroxyl radical initiator comprises a 2,2′-azobis(2-amidinopropane) salt.
3. The method of paragraph 2 wherein said 2,2′-azobis(2-amidinopropane) salt comprises 2,2′-azobis(2-amidinopropane) dihydrochloride.
4. The method of paragraph 1, further comprising comparing said antioxidant capacity of said test compound to an antioxidant capacity of a standard compound, wherein said antioxidant capacity of a standard compound is generated by the steps of:
   (a) contacting a cultured cell with 2′,7′-dichlorofluorescin diacetate in the presence of a standard compound, wherein the 2′,7′-dichlorofluorescin diacetate enters the cell and is cleaved to 2′,7′-dichlorofluorescin;
   (b) contacting the cell with a peroxyl radical initiator;
   (c) measuring fluorescence in an emission wavelength of 2′,7′dichlorofluorescein at a plurality of time points; and
   (d) determining the area-under-the-curve of a graph plotting 2′,7′-dichlorofluorescein diacetate fluorescence vs. time for the cultured cell in the presence the standard compound.
5. The method of paragraph 4, wherein said standard compound is selected from the group consisting of quercetin, galangin, EGCG and kaempferol.
6. The method of paragraph 1 wherein said emission wavelength is 538 nm.
7. The method of paragraph 1 wherein said test compound is produced by a plant.
8. The method of paragraph 7 wherein said test compound is a phytochemical.
9. The method of paragraph 1 wherein said cultured cell is a eukaryotic cell.
10. The method of paragraph 9 wherein said eukaryotic cell is a human cell.
11. The method of paragraph 9 wherein said eukaryotic cell is a cell of a human cell line.
12. The method of paragraph 11 wherein said human cell line is HepG2.
13. The method of paragraph 1, further comprising the step of washing the cultured cell prior to the step of contacting the cell with the peroxyl initiator and comparing antioxidant activity data derived from washed cells with antioxidant activity data derived from unwashed cells.
14. A method of predicting in vivo antioxidant capacity of a compound, the method comprising the steps of:
   a) contacting a first cultured cell with 2′,7′-dichlorofluorescin diacetate, in the presence of a test compound to form a first mixture,
   b) contacting a second cultured cell with 2′,7′-dichlorofluorescin diacetate, in the absence of said test compound to form a second mixture, wherein said 2′,7′-dichlorofluorescin diacetate enters said first and said second cells and is cleaved therein to 2′,7′-dichlorofluorescin;
   c) contacting said first and second mixtures with a peroxyl radical initiator; and
   d) measuring fluorescence in an emission wavelength of 2′,7′-dichlorofluorescein at a plurality of time points in said first and said second mixtures,
   e) determining area-under-the-curve of a graph plotting 2′,7′-dichlorofluorescein diacetate fluorescence vs. time for said first and second mixtures,
      wherein a decrease in said area-under-the-curve at an emission wavelength of 2′,7′-dichlorofluorescein in said first mixture, relative to said area-under-the-curve in said second mixture provides a prediction of in vivo antioxidant capacity of said test compound.
15. The method of paragraph 14, wherein said peroxyl radical initiator comprises a 2,2′-azobis(2-amidinopropane) salt.
16. The method of paragraph 14 wherein said 2,2′-azobis(2-amidinopropane) salt comprises 2,2′-azobis(2-amidinopropane) dihydrochloride.
17. The method of paragraph 14 wherein said emission wavelength is 538 nm.
18. The method of paragraph 14 wherein said cultured cell is a eukaryotic cell.
19. The method of paragraph 18 wherein said eukaryotic cell is a human cell.
20. The method of paragraph 19 wherein said eukaryotic cell is a cell of a human cell line.
21. The method of paragraph 20 wherein said human cell line is HepG2.
22. The method of paragraph 14 wherein said in vivo antioxidant capacity is compared to an in vivo antioxidant capacity of a standard compound, wherein said in vivo antioxidant capacity of a standard compound is generated by the steps of:
   (a) contacting a cultured cell with 2′,7′-dichlorofluorescin diacetate in the presence of a standard compound, wherein the 2′,7′-dichlorofluorescin diacetate enters the cell and is cleaved to 2′,7′-dichlorofluorescin;
   (b) contacting the cell with a peroxyl radical initiator;
   (c) measuring fluorescence in an emission wavelength of 2′,7′dichlorofluorescein at a plurality of time points; and
   (d) determining the area-under-the-curve of a graph plotting 2′,7′-dichlorofluorescein diacetate fluorescence vs. time for the cultured cell in the presence the standard compound.
23. The method of paragraph 22, wherein said standard compound is selected from the group consisting of quercetin, galangin, EGCG and kaempferol.
24. The method of paragraph 14, further comprising a step of washing said cultured cell prior to the step of contacting said first and second cells with said peroxyl initiator and comparing antioxidant activity data derived from washed cells with antioxidant activity data derived from unwashed cells.
25. A kit for measuring the antioxidant capacity of a compound, the kit comprising:
   a) 2′,7′-dichlorofluorescin diacetate;
   b) a peroxyl radical initiator;
   c) a standard;
   d) computer readable medium comprising instructions for determining antioxidant capacity of a test compound, and
   e) packaging materials therefor.
26. The kit of paragraph 25, further comprising a viable eukaryotic cell.
27. The kit of paragraph 26, wherein said viable eukaryotic cell is a human cell.
28. The kit of paragraph 27, wherein said human cell is a cell of a cell line.
29. The kit of paragraph 25, wherein said peroxyl radical initiator comprises a 2,2′-azobis(2-amidinopropane) salt.
30. The kit of paragraph 29 wherein said 2,2′-azobis(2-amidinopropane) salt comprises 2,2′-azobis(2-amidinopropane) dihydrochloride.
31. The kit of paragraph 29, wherein said standard is selected from the group consisting of quercetin, galangin, EGCG and kaempferol.
32. A method for determining an absolute value of antioxidant activity for a test compound, the method comprising the steps of:
   a) contacting a first cultured cell with 2′,7′-dichlorofluorescin diacetate, in the presence of a test compound,
   b) contacting a second cultured cell with 2′,7′-dichlorofluorescin diacetate, in the absence of said test compound, wherein said 2′,7′-dichlorofluorescin diacetate enters said first and second cells and is cleaved therein to 2′,7′-dichlorofluorescin;
   c) contacting said first and second cells with a peroxyl radical initiator; and
   d) measuring fluorescence in an emission wavelength of 2′,7′-dichlorofluorescein at a plurality of time points in said first and second cultured cells,
   e) determining the ratio of area-under-the-curve of a graph plotting 2′,7′-dichlorofluorescein diacetate fluorescence vs. time for said first and second cultured cells,
   f) normalizing the ratio of area-under-the-curve of step (e) to area-under-the-curve of a graph plotting 2′,7′-dichlorofluorescein diacetate fluorescence vs. time for a standard compound.
33. The method of paragraph 32, wherein said area-under-the-curve for said standard compound is generated by the steps of:
   a) contacting a cultured cell with 2′,7′-dichlorofluorescin diacetate in the presence of a standard compound, wherein said 2′,7′-dichlorofluorescin diacetate enters said cell and is cleaved to 2′,7′-dichlorofluorescin;
   b) contacting said cell with a peroxyl radical initiator; and
   c) measuring fluorescence in an emission wavelength of 2′,7′-dichlorofluorescein at a plurality of time points,
   d) determining the area-under-the-curve of a graph plotting 2′,7′-dichlorofluorescein diacetate fluorescence vs. time for said cultured cell in the presence said standard compound.
34. The method of paragraph 32, wherein an absolute value of antioxidant activity is determined for a test compound by applying the values obtained in paragraph 31 to Equation (1)

$$CAA abs = \frac{( 1 - ( \int SA / \int CA ) )}{( 1 - ( \int Sq / \int CA ) )} \qquad Equation ( 1 )$$

wherein ∫SA is the area-under-the-curve for fluorescence vs. time of said test compound, ∫CA is the area-under-the-curve for fluorescence vs. time in the absence of said test compound, and ∫S_{q} is the area-under-the-curve for fluorescence vs. time of said standard compound, and wherein CAA_{abs} is the absolute value of antioxidant activity for a test compound.
35. The method of paragraph 32 wherein said peroxyl radical initiator comprises a 2,2′-azobis(2-amidinopropane) salt.
36. The method of paragraph 35 wherein said 2,2′-azobis(2-amidinopropane) salt comprises 2,2′-azobis(2-amidinopropane) dihydrochloride.
37. The method of paragraph 33, wherein said standard compound is selected from the group consisting of quercetin, galangin, EGCG and kaempferol.
38. The method of paragraph 32 wherein said emission wavelength is 538 nm.
39. The method of paragraph 32 wherein said test compound is produced by a plant.
40. The method of paragraph 32 wherein said test compound is a phytochemical.
41. The method of paragraph 32 wherein said cultured cell is a eukaryotic cell.
42. The method of paragraph 41 wherein said eukaryotic cell is a human cell.
43. The method of paragraph 41 wherein said eukaryotic cell is a cell of a human cell line.
44. The method of paragraph 43 wherein said human cell line is HepG2.
45. The method of paragraph 32, further comprising a step of washing said cultured cell prior to the step of contacting said cell with said peroxyl initiator and comparing antioxidant activity data derived from washed cells with antioxidant activity data derived from unwashed cells.
46. A computer-readable medium comprising instructions for obtaining an absolute antioxidant value from fluorescence measured at a plurality of time points, the medium comprising:
   (a) instructions for receiving a plurality of fluorescence values, the values representing fluorescence at a plurality of time points for a cultured cell in the presence and absence of a test compound;
   (b) instructions for receiving a plurality of fluorescence values, the values representing fluorescence at a plurality of time points for a cultured cell in the presence of a standard compound;
   (c) instructions for calculating an absolute antioxidant value, CAA_{abs}, for said test compound, said instructions comprising applying the values received according to instructions (a) and (b) to the relationship of Equation (1)

$$CAA abs = \frac{( 1 - ( \int SA / \int CA ) )}{( 1 - ( \int Sq / \int CA ) )} \qquad Equation ( 1 )$$

wherein ∫SA is the area-under-the-curve for fluorescence vs. time of said test compound, ∫CA is the area-under-the-curve for fluorescence vs. time in the absence of said test compound, and ∫S_{q} is the area-under-the-curve for fluorescence vs. time of said standard compound; and
(d) instructions for transmitting a value for CAA_{abs} to an output device.

### EXAMPLES

### Example 1

### Cellular Antioxidant Activity Assay for Assessing Antioxidants, Food and Dietary Supplements

### A. Materials and Methods

### Chemicals

Folin-Ciocalteu reagent, 2′,7′-dichlorofluorescin diacetate (DCFH-DA), ethanol, glutaraldehyde, methylene blue, ascorbic acid, caffeic acid, (+)-catechin, (−)-epicatechin, (−)-epigallocatechin gallate (EGCG), ferulic acid, kaempferol, luteolin, myricetin, phloretin, quercetin dihydrate, resveratrol, and taxifolin were purchased from Sigma-Aldrich, Inc. (St. Louis, Mo.). Gallic acid was obtained from ICN Biomedicals, Inc. (Aurora, Ohio). Dimethyl sulfoxide and acetic acid were obtained from Fisher Scientific (Pittsburgh, Pa.) and 2,2′-azobis (2-amidinopropane) dihydrochloride (ABAP) was purchased from Wako Chemicals USA, Inc. (Richmond, Va.). Sodium carbonate, acetone, and methanol were obtained from Mallinckrodt Baker, Inc. (Phillipsburg, N.J.). The HepG2 cells were obtained from the American Type Culture Collection (ATCC) (Rockville, Md.). Williams' Medium E (WME) and Hanks' Balanced Salt Solution (HBSS) were purchased from Gibco Life Technologies (Grand Island, N.Y.). Fetal bovine serum (FBS) was obtained from Atlanta Biologicals (Lawrenceville, Ga.).

### Fruit Samples

Wild blueberries were obtained from the Wild Blueberry Association of North America (Orono, Me.). Red Delicious apples were obtained from Cornell Orchards (Ithaca, N.Y.). Green and red seedless table grapes and frozen cranberries were purchased at a local supermarket (Ithaca, N.Y.).

### Fruit Extractions

Extracts were obtained from the fruits using 80% acetone, as described previously (6).

### Determination of Total Phenolic Content

The total phenolic contents of the fruit extracts were determined using the Folin-Ciocalteu colorimetric method (26), as modified by our laboratory (27, 28). Results were expressed as mean mol gallic acid equivalents (GAE)/100 g fresh fruit±SD for three replicates.

### Preparation of Chemical and Fruit Sample Solutions

A 20 mM stock solution of DCFH-DA in methanol was prepared, aliquoted, and stored at −20° C. A 200 mM ABAP stock solution was prepared and aliquots were stored at −40° C. Working phytochemical and fruit extract solutions were prepared just prior to use. Caffeic acid, (+)-catechin, EGCG, (−)-epicatechin, ferulic acid, gallic acid, kaempferol, myricetin, phloretin, resveratrol, and taxifolin were dissolved in ethanol, luteolin was dissolved in methanol, and quercetin was dissolved in dimethyl sulfoxide before further dilution in treatment medium (WME with 2 mM L-glutamine and 10 mM Hepes). Fruit extracts were diluted in treatment medium. Final treatment solutions contained less than 2% solvent and there was no cytotoxicity to HepG2 cells at those concentrations.

### Cell Culture

HepG2 cells were grown in growth medium (WME supplemented with 5% FBS, 10 mM Hepes, 2 mM L-glutamine, 5 μg/mL insulin, 0.05 μg/mL hydrocortisone, 50 units/mL penicillin, 50 μg/mL streptomycin, and 100 μg/mL gentamycin) and were maintained at 37° C. and 5% CO2 as described previously (Wolfe, K. L and Liu, R. H. (2007), supra). Cells used in this study were between passages 12 and 32.

### Cytotoxicity

Cytotoxicity was measured using the method of Oliver et al. (31) with modifications by our laboratory (32). HepG2 cells were seeded at 4×104/well on a 96-well plate in 100 μL growth medium and incubated for 24 h at 37° C. The medium was removed and the cells were washed with PBS. Treatments of fruit extracts or antioxidant compounds in 100 μL treatment medium (Williams' Medium E supplemented with 2 mM L-glutamine and 10 mM Hepes) were applied to the cells and the plates were incubated at 37° C. for 24 h. The treatment medium was removed and the cells were washed with PBS. A volume of 50 μL/well methylene blue staining solution (98% HBSS, 0.67% glutaraldehyde, 0.6% methylene blue) was applied to each well and the plate was incubated at 37° C. for 1 h. The dye was removed and the plate was immersed in fresh deionized water three times, or until the water was clear. The water was tapped out of the wells and the plate was allowed to air-dry briefly before 100 μL elution solution (49% PBS, 50% ethanol, 1% acetic acid) was added to each well. The microplate was placed on a bench-top shaker for 20 minutes to allow uniform elution. The absorbance was read at 570 nm with blank subtraction using the MRX II DYNEX spectrophotometer (DYNEX Inc., Chantilly, Va.). Concentrations of pure compounds or fruit extracts that decreased the absorbance by more than 10% when compared to the control were considered to be cytotoxic.

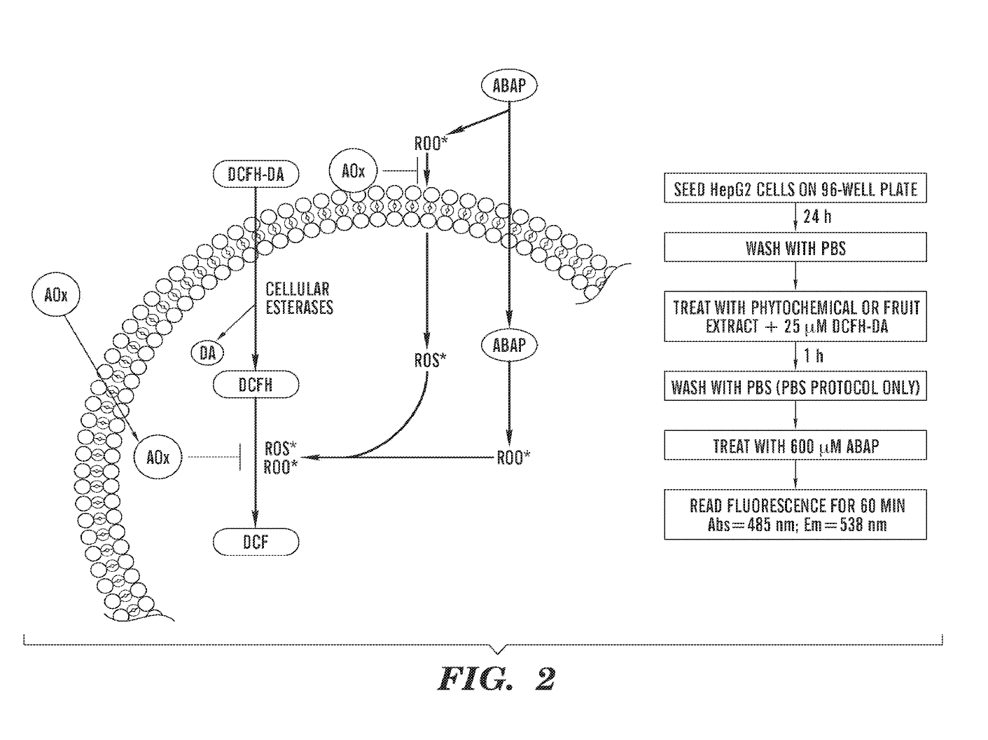

### Cellular Antioxidant Activity of Pure Phytochemicals and Fruit Extracts (FIG. 2)

Human hepatocellular carcinoma HepG2 cells were seeded at a density of 6×104/well on a 96-well microplate in 100 μL growth medium/well. The outside wells of the plate were not used as there was much more variation from them than from the inner wells. Twenty-four hours after seeding the growth medium was removed and the wells were washed with PBS. Triplicate wells were treated for 1 h with 100 μL pure phytochemical compounds or fruit extracts plus 25 μM DCFH-DA dissolved in treatment medium. When a PBS wash was utilized, wells were then washed with 100 μL PBS. Then 600 μM ABAP was applied to the cells in 100 μL HBSS and the 96-well microplate was placed into Fluoroskan Ascent FL plate-reader (ThermoLabsystems, Franklin, Mass.) at 37° C. Emission at 538 nm was measured with excitation at 485 nm every 5 min for 1 h. Each plate included triplicate control and blank wells: control wells contained cells treated with DCFH-DA and oxidant; blank wells contained cells treated with dye and HBSS without oxidant.

### Quantification of Cellular Antioxidant Activity (CAA)

After blank subtraction from the fluorescence readings, the area under the curve of fluorescence versus time was integrated to calculate the cellular antioxidant activity (CAA) value at each concentration of pure phytochemical compound or fruit extract as follows:

CAA unit=100−(∫SA/∫CA)×100

where ∫SA is the integrated area under the sample fluorescence versus time curve and ∫CA is the integrated area from the control curve. The median effective dose (EC50) was determined for the pure phytochemical compounds and fruit extracts from the median effect plot of log (fa/fu) vs. log (dose), where fa is the fraction affected and fu is the fraction unaffected by the treatment. To quantify intraexperimental variation, the EC50 values were stated as mean±SD for triplicate sets of data obtained from the same experiment. Interexperimental variation was obtained for some representative pure phytochemical compounds and fruit extracts by averaging the fluorescence values from triplicate wells in each trial to obtain one EC50 value per experiment and calculating the mean±SD for at least four trials. In each experiment, quercetin was used as a standard and cellular antioxidant activities for pure phytochemical compounds were expressed as mol quercetin equivalents (QE)/100 mol compound, while for fruit extracts they were expressed as mol QE/100 g fruit. In order to compare the antioxidant quality of different fruits, cellular antioxidant activity (CAA) was also calculated as mol QE/100 mol total phenolics.

### Statistical Analyses

All results were presented as mean±SD. Comparisons between two means were performed using unpaired Student's t-tests. When there were more than two means, differences were detected by ANOVA followed by multiple comparisons using Fisher's least significant difference test. Differences were considered to be significant when p<0.05.

### B. Total Phenolic Contents of Fruit Extracts

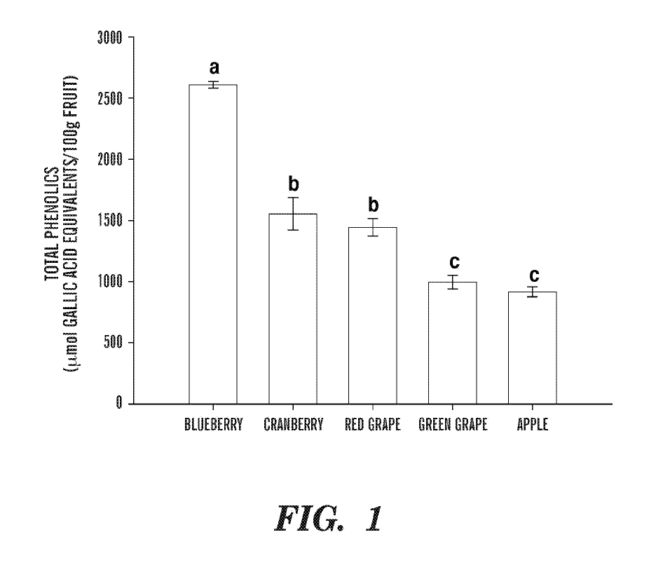

In order to characterize the fruit extracts used in the cellular antioxidant activity assay, the total phenolic contents of the fruits were quantified (FIG. 1). Blueberry contained the most phenolics with 2609±28 μmol GAE/100 g fresh fruit, followed by cranberry (1554±134 μmol GAE/100 g), red grape (1443±72 μmol GAE/100 g), green grape (994±56 μmol GAE/100 g), and apple (916±41 μmol GAE/100 g).

### C. Cellular Antioxidant Activity (CAA)

The proposed principle of the CAA assay is shown in FIG. 2. Based on the optimization trials (data not shown), a concentration of 25 μM DCFH-DA was used because lower levels did not yield consistent fluorescence measurements and higher concentrations decreased the sensitivity of the assay. ABAP caused oxidation of DCFH-DA in a dose-response manner up to a dose of 2 mM (data not shown). The treatment level of 600 μM was chosen because it yielded adequate fluorescence readings while inducing a reasonable level of oxidation that could be inhibited by many phytochemicals and fruit extracts. The kinetics of DCFH oxidation in HepG2 cells by peroxyl radicals generated from ABAP is shown in FIG. 3. The increase in fluorescence from DCF formation was inhibited by pure phytochemical compounds and fruit extracts in a dose-dependent manner, as demonstrated by the curves generated from cells treated with quercetin (FIGS. 3A, 3B), gallic acid (FIGS. 3C, 3D), and blueberry extracts (FIGS. 3E, 3F). Inhibition of oxidation was seen when no PBS wash was done between antioxidant and ABAP treatments (FIGS. 3A, 3C, 3E) and when a PBS wash was performed (FIGS. 3B, 3D, 3F).

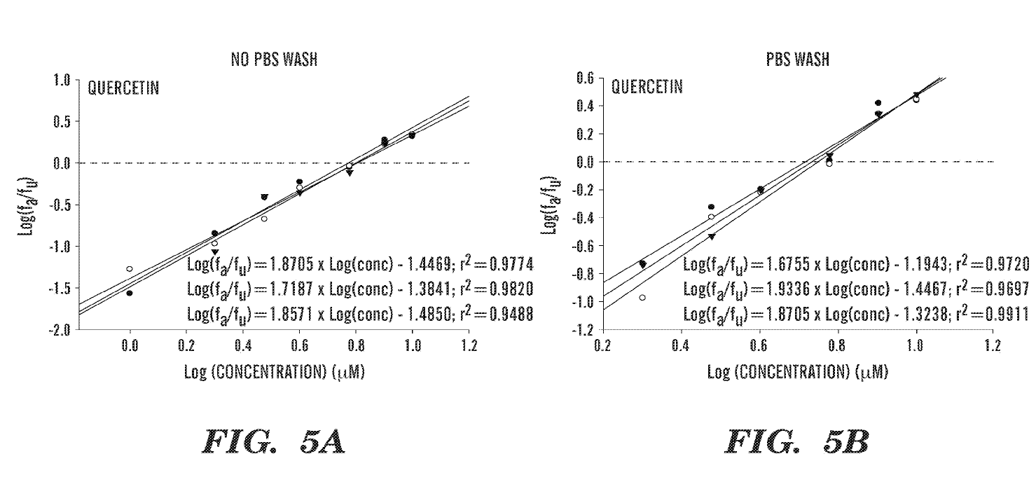

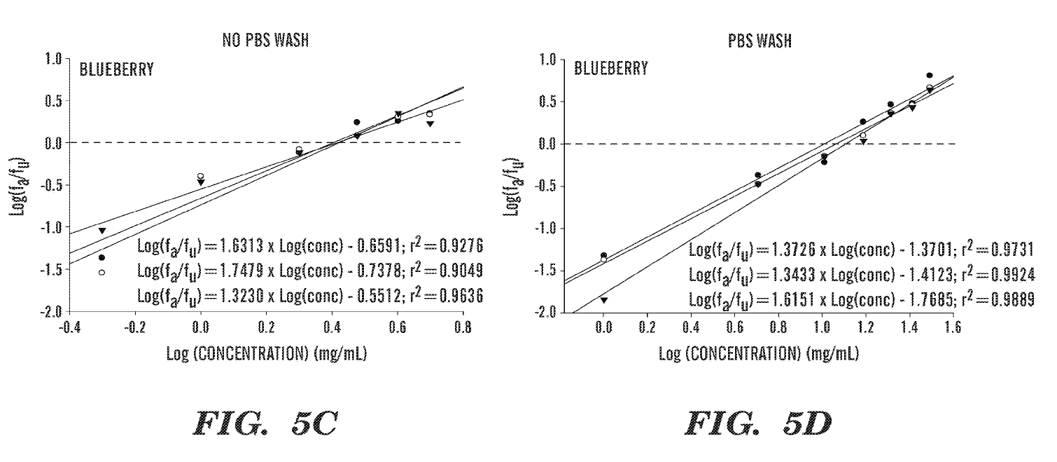

In order to calculate the EC₅₀, the dose-response curve from the ratio of the area under the curve of the sample to that of the control, and the median effect curve were plotted for each sample. The dose-response curves and median effect plots generated from the data presented from quercetin and blueberry extracts in FIG. 3 are shown in FIG. 4 and FIG. 5, respectively. The EC₅₀ is the concentration at which fₐ/fᵤ=1 (i.e., CAA unit=50), as calculated from the linear regression of the median effect curve.

The EC₅₀ values of CAA for pure phytochemical compounds and fruit extracts are listed in Table 1 along with their cytotoxic concentrations. The values presented are from triplicate samples in the same experiment and the coefficient of variation (CV) represents intraexperimental variation. When more than one experiment was performed for the sample, representative results from one trial were presented. In the protocol involving no PBS wash between antioxidant and ABAP treatments (no PBS wash) for the pure phytochemical compounds, quercetin was the most efficacious antioxidant, followed by kaempferol, EGCG, myricetin, luteolin, gallic acid, ascorbic acid, caffeic acid, and catechin (Table 1). Epicatechin and ferulic acid had low activity within the doses tested and their EC₅₀ values could not be calculated. Phloretin, resveratrol, and taxifolin had activity only at doses much higher than their cytotoxic concentrations.

For those experiments including a PBS wash between treatments, the order of efficacies was similar to that obtained from the no PBS wash protocol, except that the CAA activity from ascorbic acid and catechin was low and EC₅₀ values of CAA could not be calculated (Table 1). Quercetin, kaempferol, and luteolin had slightly higher EC₅₀ concentrations when a PBS wash was done between treatments (p<0.05). Myricetin had similar EC₅₀ values in each of the two protocols (p>0.05), as did EGCG (p>0.05). Gallic acid and caffeic acid had much lower activity when a PBS wash was performed compared to that in the no PBS wash protocol (p<0.05). The intraexperimental coefficient of variation (CV %) for the pure compounds was under 10% when a PBS wash was utilized, and modestly higher when a PBS wash was not done (Table 1).

The EC₅₀ values of CAA for the fruit extracts are presented in Table 1. Blueberry was the most effective at inhibiting peroxyl radical-induced DCFH oxidation, followed by cranberry, apple, red grape, and green grape. The order of efficacy was the same with or without a PBS wash between fruit extracts and ABAP treatments. The fruit extracts all had lower EC₅₀ values in the no PBS protocol than in the PBS wash protocol (p<0.05). The intraexperimental CV (%) ranged from 2.59 to 16.0%, with the majority of trials yielding a CV of less than 10% (Table 1).

The relationship between EC₅₀ values and total phenolic contents of fruit extracts was examined. When no PBS wash was employed between treatments, the EC₅₀ values for CAA were not significantly correlated to total phenolic contents in fruits (R²=0.450; p=0.215). EC₅₀ values were weakly correlated to total phenolic contents (R²=0.830; p=0.032) in fruits when a PBS wash was done.

The reproducibility of EC₅₀ values of CAA from similar experiments performed on different days (interexperimental variation) was evaluated for representative compounds tested using the no PBS wash and PBS wash protocols (Table 2 and Table 3). For pure compounds with no PBS wash performed between treatments, the CV (%) of interexperimental variation for quercetin and gallic acid were 6% and 17%, respectively (Table 2). The CV of the no PBS wash interexperimental variation for blueberry was 22% (Table 2). When a PBS wash was done between antioxidant and oxidant treatments, the CV for quercetin, gallic acid, and blueberry extracts were 11.3, 11.4, and 7.56%, respectively (Table 3).

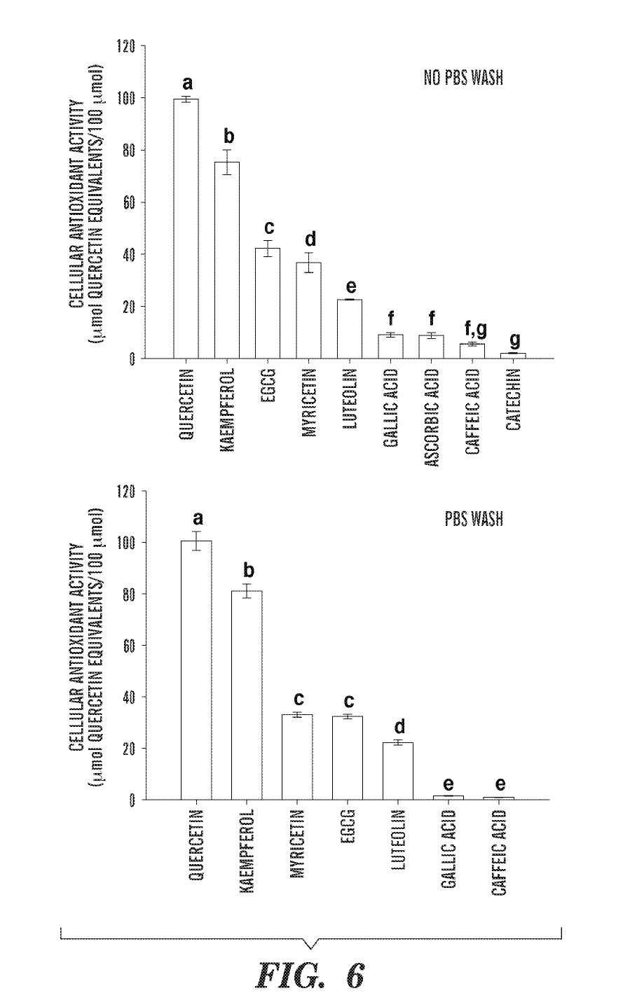

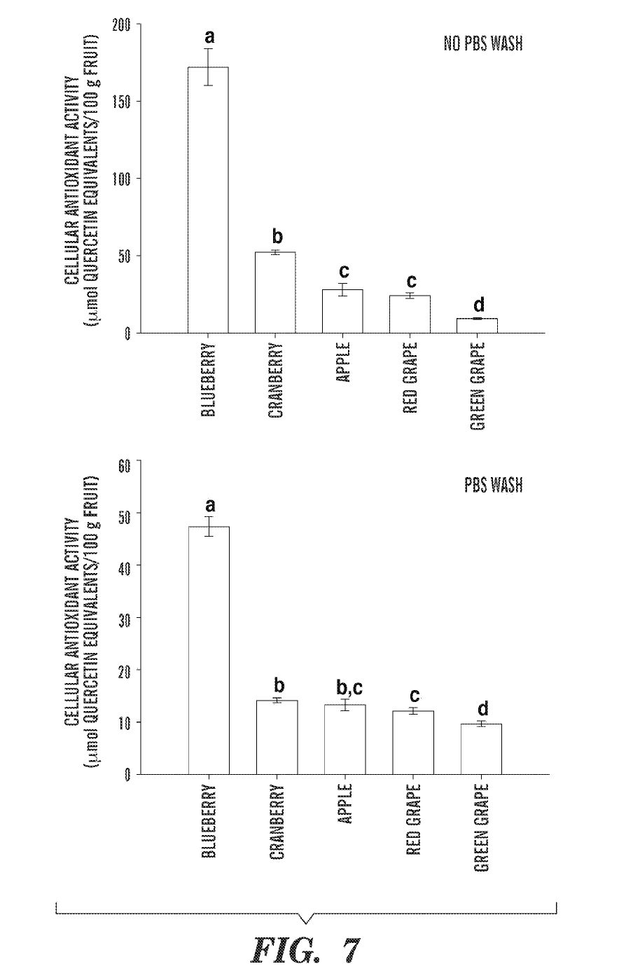

The EC₅₀ values were converted to cellular antioxidant activity (CAA) values, expressed as μmol QE/100 μmol compound for pure antioxidant compounds (FIG. 6) and μmol QE/100 g fresh fruit for fruit extracts (FIG. 7). When no PBS wash was done between antioxidant and ABAP treatments, quercetin had the highest CAA value (p<0.05), followed by kaempferol (75.3±4.7 μmol QE/100 μmol), EGCG (42.2±3.1), myricetin (36.8±3.8), and luteolin (22.6±0.2), which were all significantly different (p<0.05). The CAA values for gallic acid, ascorbic acid, and caffeic acid were not significantly different (9.08±0.95, 8.84±1.18, and 5.59±0.70, respectively) (p>0.05), and catechin's CAA value was similar to caffeic acid's at 2.03±0.24 μmol QE/100 μmol (p>0.05).

When the HepG2 cells were washed with PBS between treatments, the order of activity was nearly the same: quercetin>kaempferol (81.1±2.7 μmol QE/100 mol)>myricetin (33.1±1.0)=EGCG (32.3±0.9)>luteolin (22.2±1.0)>gallic acid (1.53±0.12)=caffeic acid (0.997±0.074) at a significance level of p<0.05.

For the fruit extracts in the no PBS wash group, blueberry had the highest CAA value (171±12 μmol QE/100 g) (p<0.05). The remaining fruits had activity in the order of cranberry (52.1±1.3)>apple (28.1±4.1)=red grape (24.1±1.7)>green grape (9.39±0.49) at a significance level p<0.05. Again, in the PBS wash protocol, blueberry had the greatest activity (47±1.9 μmol QE/100 g) (p<0.05), followed by cranberry (14.2±0.5) (p<0.05). The CAA value of apple (13.3±1.1) was not significantly different from that of cranberry (p>0.05), and the CAA value of red grape (12.1±0.6) was similar to that of apple (p>0.05). Green grape had the lowest CAA value (9.67±0.57) (p<0.05) when a PBS wash was performed between treatments.

In order to compare the antioxidant quality of different fruits, cellular antioxidant activity (CAA) values can be expressed as μmol QE per 100 μmol total phenolics (Table 4). This value makes it possible to compare the antioxidant quality of the total phytochemicals in whole foods compared to pure phytochemical compounds. In the no PBS wash protocol, blueberry exhibited the highest antioxidant quality (8.70±0.09 μmol QE/100 μmol total phenolics) (p<0.05), followed by similar values from cranberry (3.36±0.09) and apple (3.07±0.45), then red grape (1.67±0.12) and green grape (1.04±0.05). These values are comparable to the activities of 100 μmol gallic acid, ascorbic acid, caffeic acid, and catechin (FIG. 6). When a PBS wash was done, the antioxidant quality values were 1.82±0.07 μmol QE/100 μmol total phenolics for blueberry, 1.45±0.12 for apple, 0.973±0.057 for green grape, 0.914±0.030 for cranberry, and 0.839±0.044 for red grape, comparable to the efficacies of 100 μmol gallic acid or caffeic acid (FIG. 6). There were no significant differences between the antioxidant quality of cranberry and green grape or cranberry and red grape (p>0.05) in the PBS wash protocol.

### D. Principle of the Cellular Antioxidant Activity (CAA) Assay

Described herein is a method to measure antioxidant activity of a test compound in cell culture. As indicated at the First International Congress on Antioxidant Methods, there is a need for more appropriate methods to evaluate the antioxidant activity of dietary supplements, phytochemicals, and foods than the chemistry methods in common usage (Liu, R. H. and Finley, J., (2005), supra). The cellular antioxidant activity assay (CAA) addresses this need for a biologically relevant protocol. In this method (FIG. 2) the probe, DCFH-DA, is taken up by HepG2 human hepatocarcinoma cells and deacetylated to DCFH. Peroxyl radicals generated from ABAP lead to the oxidation of DCFH to fluorescent DCF, and the level of fluorescence measured upon excitation is proportional to the level of oxidation. Pure phytochemical compounds and fruit extracts quench peroxyl radicals and inhibit the generation of DCF. Thus, the CAA assay uses the ability of peroxyl radicals, reactive products of lipid oxidation, to induce the formation of a fluorescent oxidative stress indicator in the cell culture and measures the prevention of oxidation by antioxidants. In addition, the CAA assay uses a measurement of fluorescence over time (i.e., area-under-the-curve), which permits slight variations among assays to be reduced, thus determining a more accurate measure of antioxidant capacity. Similarly, the use of a standard compound in the CAA assay permits the comparison of the antioxidant activities of (1) unrelated compounds, (2) results from different laboratories, and/or (3) results measured at different times or by different users. The methods described herein are necessary for reducing variability among antioxidant capacity measurements and permitting the standardization of antioxidant capacity measurements by normalizing to a known antioxidant standard.

### E. DCFH-DA as an Indicator of Oxidation

Keston and Brandt (Keston, A. S. and Brandt, R., (1965) Anal. Biochem. 11:1-5) first reported the use of DCFH oxidation to measure hydrogen peroxide levels in a cell-free system. DCFH-DA was first “activated” by alkali removal of the diacetate moiety. When added to hydrogen peroxide and peroxidase solutions, DCFH was oxidized to form fluorescent DCF and the fluorescence measurements were proportional to the concentration of hydrogen peroxide. Several years later an assay to measure respiratory burst H₂O₂ in phorbol myristate acetate (PMA)-stimulated polymorphonuclear leukocytes was developed (Bass, D. A.; et al (1983) J. Immunol. 130(4):1910-7). Cells loaded with DCFH-DA fluoresced after PMA stimulation and the fluorescence could be quantified by flow cytometry. A DCFH-DA oxidation mechanism in cells was proposed: non-polar DCFH-DA diffused through the cell membrane and once within the cell it was deacetylated by cellular esterases, forming DCFH, which was trapped within the cell due to its more polar nature. H₂O₂ generated by PMA stimulation, possibly in combination with cellular peroxidases, then oxidized DCFH to DCF, a polar fluorescent compound that was also trapped with the cell. Spontaneous deacetylation of DCFH-DA does not seem to be a problem, as it is slow under cell-free conditions (Adom, K. K, et al (2005), supra; Royall, J. A. and Ischiropoulos, H., (1993) Arch. Biochem. Biophys. 302(2):348-55). Cellular uptake of DCFH-DA is rapid and final concentrations are relatively stable, as cultured bovine aorta endothelial cells exposed to 11 μM DCFH-DA in the medium reached maximum intracellular levels of the probe within 15 minutes and the level remained constant for one hour (Royall, J. A. and Ischiropoulos, H., (1993), supra).

In addition to H₂O₂, various other species have been found to oxidize DCFH to DCF in cell culture. In PC12, rat neuroendocrine cells, DCF can be generated from DCFH by treatment with peroxynitrite (ONOO⁻), nitric oxide (NO.), dopamine, peroxyl radicals, and H₂O₂ (Wang, H and Joseph, J. A., (1999) Free Radic. Biol. Med. 27(5-6):612-6). Xanthine oxidase, ferrous iron, superoxide, and hydroxyl radicals have also been implicated in DCFH oxidation in renal epithelial cells (Scott, J. A.; et al (1988) Free Radic. Biol. Med. 4(2):79-83). In neutrophils, DCF was generated from DCFH by Arochlor A1242 (a polychlorinated biphenyl mixture that induces respiratory burst), H₂O₂, nitric oxide, and FeSO₄ (Myhre, O.; et al (2003) Biochem. Pharmacol. 65(10):1575-82). DCFH-DA has also been used as an indicator to measure oxidative stress due to exposure to irradiation in MCF10 human breast epithelial cells (Wan, X. S.; et al (2005) Radiat. Res. 163(4):364-8; Wan, X. S.; et al (2003) Radiat. Res. 160 (6):622-30). The wide array of reactive oxygen species (ROS) that are able to oxidize DCFH to fluorescent DCF make it an attractive tool to measure general oxidative stress in cells.

There are a number of potential problems with the use of DCF as an indicator of oxidizing species. Exposure of DCFH-loaded cells to light should be minimized because DCF in the presence of reducing agents was photo-reduced under conditions of visible irradiation (Marchesi, E.; et al (1999) Free Radic. Biol. Med. 26(1-2):148-61). The resulting free radicals in the presence of oxygen can be generated continuously, and contribute to oxidation. DCFH and DCF also may not be trapped intracellularly, as generally thought. When endothelial cells previously exposed to DCFH-DA were exposed to medium free of DCFH-DA, the levels of DCFH and DCF decreased intracellularly and increased extracellularly (Royall, J. A. and Ischiropoulos, H., (1993), supra). Leakage of DCFH from mouse neuroblastoma N18 cells was also reported and it was suggested that subsequent treatments should occur as quickly as possible after loading cells with the probe (Sawada, G. A.; et al (1996) Cytometry 25(3):254-62). Finally, DCFH oxidation decreased with increasing reduced glutathione levels in Saccharomyces cereviseae cells, showing that cellular antioxidant status can influence DCF response (Jakubowski, W. and Bartosz, G., (2000) Cell Biol. Int. 24(10):757-60). Despite potential misinterpretation of results due to the above factors, DCFH-DA is useful as an indicator of general cellular oxidation levels in a well-defined protocol.

### F. ABAP as a Generator of Peroxyl Radicals

ABAP (2,2′-azobis(2-amidinopropane)) is an azo radical initiator used as a oxidant source in many antioxidant activity protocols (Cao, G.; et al (1993), supra; Ghiselli, A.; et al (1995), supra; Adom, K. K et al (2005), supra; Chu, Y. F. and Liu, R. H., (2004) J. Agric. Food Chem. 52(22):6818-23; Regoli, F. and Winston, G. W., (1999) Toxicol. Appl. Pharmacol. 156(2):96-105). It thermally decomposes to generate nitrogen gas and two carbon-centered radicals. These radicals can then react with each other or form peroxyl radicals by reacting with molecular oxygen. The half-life of ABAP at 37° C. in neutral water is about 175 h, and the rate of radical generation is constant for the first few hours (Niki, E., (1990) Methods. Enzymol. 186: 100-8). The peroxyl radicals are generated in the aqueous phase, where they can cause chain reactions and damage organs indiscriminately in vivo. ABAP has been shown to induce the formation of DCF in cell culture in a dose-dependent manner (Wang, H., et al (1999), supra).

The use of azo compounds, such as ABAP, to form peroxyl radicals in biomimetic experiments has been criticized (Frankel, E. N.; Meyer, A. S (2000), supra; Paul, T.; et al (2000) Biochemistry 39(14):4129-35). In particular, azo initiators form an abundance of peroxyl radicals that do not have time to perpetuate chain reactions in the time employed in antioxidant activity assays, so their use overemphasizes the initiation phase of lipid oxidation and largely ignores the propagation and decomposition phases (Frankel, E. N.; Meyer, A. S (2000), supra). Although ABAP is not a physiologically relevant compound, peroxyl radicals, which are generated by ABAP decomposition, are a major type of ROS in vivo, so it is a good tool for the examination of peroxyl radical-induced damage to membranes and other biological molecules and for studying the inhibition of these effects by antioxidants (Niki, E., (1990), supra).

### G. Suggested Standards for CAA Assay

To be able to compare data in the literature from different laboratories, the CAA method should be standardized. For the methods described herein, it is strongly recommended that quercetin be used as a standard in this new assay for quantifying cellular antioxidant activity for the following reasons: 1) quercetin has high CAA activity compared to other phytochemicals (FIG. 6); 2) the pure compound is easily and economically obtained; 3) quercetin and its conjugates are found widely in fruits, vegetables, and other plants; and 4) it is relatively stable. Other standards can include galangin, ECGC, and kaempherol, among others.

### H. CAA of Selected Phytochemicals and Fruits

There are two opportunities for compounds to exert their antioxidant effects in the CAA model described herein. They can act at the cell membrane and break peroxyl radical chain reactions at the cell surface, or they can be taken up by the cell and react with ROS intracellularly. Therefore, the efficiency of cellular uptake and/or membrane-binding combined with the radical scavenging activity likely dictates the efficacy of the tested compound. Among the pure phytochemicals examined in the CAA assay, quercetin, kaempferol, EGCG, myricetin, and luteolin showed the highest cellular antioxidant activities, exhibiting between 22 and 100 percent of the antioxidant activity of quercetin. These flavonoids were likely well-absorbed by the HepG2 cells, as quercetin, kaempferol, and luteolin were also shown to be absorbed and incorporated into Caco-2 cells (Yokomizo, A. and Moriwaki, M., (2006) Biosci. Biotechnol. Biochem. 70(6):1317-24.), although there was no myricetin uptake in that study and EGCG was not examined. Other phytochemicals, such as ascorbic acid, gallic acid, caffeic acid and catechin, had less than ten percent of the activity of quercetin in the CAA assay.

The physical properties of flavonoids (and presumably other classes of phytochemicals) determine their interactions with the cell membrane (Oteiza, P. I.; et al (2005) Clin. Dev. Immunol. 12(1):19-25). Hydrophobic flavonoids may become deeply embedded in membranes where they can influence membrane fluidity and break oxidative chain reactions. More polar compounds interact with membrane surfaces via hydrogen bonding, where they are able to protect membranes from external and internal oxidative stresses. There is also some evidence that uptake in vivo may be related to the polarity of the compounds because the net transfer of flavonoids across the brush border of rat small intestine was found to be related to their lipophilicity, rather than their spatial conformation (Crespy, V.; et al (2003) Am. J. Physiol. Gastrointest. Liver Physiol. 284(6):G980-8).

The hydrophobicity of compounds may be important, but it is not the only factor determining their effectiveness as antioxidants in cell culture, as there was no relationship between log P (octanol-water partitioning coefficients) and activity in this model (data not shown). This was supported by a study using PC12 cells treated with H₂O₂ which showed the effectiveness of flavonoids to decrease oxidative stress as measured by DCFH oxidation, was strongly associated with structural principles, not octanol-water partitioning behaviors (Wang, H. and Joseph, J. A., (1999) Free Radic. Biol. Med. 27(5-6):683-94). In the evaluation of quercetin and compounds structurally similar to quercetin, they found that the 3′,4′-hydroxyl groups in the B ring and a 2,3-double bond conjugated with a 4-oxo group in the C ring of quercetin conferred it with most activity against H₂O₂ oxidation. Further phenolic compounds should be tested to further elucidate structure-function relationships that exist for the CAA protocol; however, the flavonoids with a 2,3-double bond and 4-oxo group, which include quercetin, kaempferol, myricetin, and luteolin, all had high activity. It is unknown why catechin, epicatechin, ferulic acid, and resveratrol had low efficacy in this model and why EGCG had such high activity. A more comprehensive screening of a phytochemicals and their conjugates is necessary to fully determine the structural and physical properties that dictate effectiveness in the CAA assay.

Some compounds, such as quercetin, kaempferol, myricetin, EGCG, and luteolin showed little, if any, difference in antioxidant efficacy whether or not a PBS wash was done between antioxidant and ABAP treatments, as measured by EC₅₀ values for CAA. Gallic acid, ascorbic acid, caffeic acid, and catechin, on the other hand, displayed dramatically lower effects when a PBS wash was done. The comparisons in antioxidant activities using the protocols with and without a PBS wash may provide information on the degree of uptake and membrane association of the pure phytochemicals or the compounds present in the fruit extracts. When a PBS wash is employed, compounds must either be taken up by the cells or be closely associated with the cell membrane in order to have antioxidant effects, as the PBS will remove compounds that are only loosely associated with the membrane. The results infer that gallic acid, ascorbic acid, caffeic acid, and catechin adsorb more loosely to the cell membrane and are taken up less readily than the flavonols, luteolin, and EGCG.

In addition to the differences in activity using the two protocols, there were also differences in variation. When no PBS wash was done between treatments, the activity may have been higher, but the coefficient of variation (CV) also tended to be higher (Tables 1-3). This was likely due to the interaction of the samples and oxidants with other factors in the residual medium on the cells. Washing the cells with PBS removed most of the interfering medium components and increased the consistency of the results. Other sources of variation may include differences in cell characteristics due to the passage number of the cells, deviation in the actual number of cells plated or surviving between experiments, and cell clumping. In addition, “cross-talk” and variation may have been decreased by using black 96-well plates instead of the clear plates employed. Differences in the content in cellular antioxidant defenses naturally present, such as glutathione, vitamin E, cysteine, phenolic amino acids and proteins, may also contribute to the variation in CAA activity between experiments. However, using the area under the curve ratios of treated cells to controls should negate the effects of these compounds.

### I. Advantages of CAA Assay

It has been suggested that the following should be considered in choosing appropriate methods to measure antioxidant activity: physiologically relevant substrates; conditions that mimic biological systems; low levels of oxidants that represent all stages of lipid oxidation; measurement of different compounds at comparable concentrations and use of plant extracts where the phenolic composition is known; and quantify based on induction period, percent inhibition, rates of product formation/decomposition, or median effective dose (Frankel, E. N., et al (2000), supra). The cellular antioxidant activity assay presented here is believed to address many of those issues. A relatively low level of ABAP, 600 μM, is used to generate peroxyl radicals to initiate oxidation and the use of excessive levels of antioxidants was avoided. The area under the kinetic curve is employed to calculate cellular antioxidant activity, which takes into consideration both the oxidation lag time increases and degree of ROS scavenging by the antioxidants tested. The median effective dose is calculated and expression of the results in μmol quercetin equivalents relates the activities to an inexpensive and ubiquitous phytochemical with biological activity. It also allows for direct comparisons of activities of different sample types and of results from other laboratories. The use of molarity instead of mass makes comparisons of antioxidant activity of compounds with different molecular weights more valid. Expression of results in quercetin equivalents per mg phytochemical may be more accessible, but it does little to describe the relative efficacy of compounds. By describing antioxidant activity per μmol phytochemical, molecules of compounds with different molecular weights and functional groups can be compared directly.

Popular antioxidant activity/capacity assays, such as ORAC (Cao, G. et al (1993), supra), TRAP (Ghiselli, A., et al (1995), supra), TEAC (Miller, N. J. et al (1993), supra), TOSC (Winston, G. W., et al (1998), supra), PSC (Adom, K. K, et al (2005), supra) and FRAP (Benzie, I. F, et al (1996), supra), all have the limitation of the inability to represent the complexity of biological systems. They measure chemical reactions only and these reactions cannot be interpreted to represent activity in vivo, as they cannot account for the bioavailability, stability, tissue retention, or reactivity of the compounds under physiological conditions (Huang, D., et al (2005). J. Agric. Food Chem. 53(6):1841-56). Oxidation of DCFH to DCF has been used as an indicator of oxidative stress and its attenuation by phytochemicals and food extracts in cell cultures (Yokomizo, A., et al (2006), supra; Wang, H., et al (1999), supra; Eberhardt, M. V, et al (2005) J. Agric. Food Chem. 53(19):7421-31; Wan, X. S. et al (2006) Int. J. Radiat. Oncol. Biol. Phys. 64(5):1475-81), but these assays are not designed to measure antioxidant activity and there is no consistency in the protocols used. The methods described herein are unique in that they use area-under-the-curve to determine the antioxidant capacity, rather than measuring fluorescence at a single time point, thus permitting values from a CAA assay performed at one time to be directly compared to values from a CAA assay performed at a different time or even by a different user. In addition, differences exist in the cell lines, types of oxidants, media, concentrations of reagents, treatment orders and times, and oxidative stress quantification methods for the previously noted assays. In order for results to be comparable among laboratories, a standardized method should be adopted.

The importance of using a more biologically relevant model in the determination of antioxidant activity is highlighted by the differences between the results of pure chemistry assays and those based in cell culture. Of the phytochemicals tested in this model, quercetin, catechin, caffeic acid had the most activity in the ORAC assay (Ou, B., et al (2001) J. Agric. Food Chem. 49(10):4619-26); gallic acid, epicatechin, and EGCG were the most effective in the TEAC assay (Kim, D. O and Lee, C. Y., (2004) Crit. Rev. Food Sci. Nutr. 44(4):253-73); quercetin, myricetin, and kaempferol were the best using the FRAP method (Firuzi, O., et al (2005) Biochim. Biophys. Acta 1721(1-3):174-84) and EGCG, chlorogenic acid, and caffeic acid were the most efficacious in the PSC protocol (Adom, K. K., et al (2005), supra). Not only are the results different from those yielded from our cellular antioxidant activity model, but they are also different from each other. Similarly, there is no consistency in the order of antioxidant activity of fruit extracts in different assays. In this model, the order of antioxidant activity was blueberry>cranberry>apple≈red grape>green grape. In the PSC and TOSC assays, the order of efficacy cranberry>apple>red grape was the same (Sun, J, et al (2002), supra; Adom, K. K., et al (2005), supra). However, oxidation of LDL by cupric ions was prevented best by cranberry, then blueberry, apple, green grape, and red grape (Vinson, J. A., et al (2001) Agric. Food Chem. 49(11):5315-21); and in the ORAC assay, red grape had higher activity than apple (Wang, H, et al (1996) J. Agric. Food Chem. 44(3):701-705). In a study that compared results from a cell-based model to those from a chemistry model using the same samples, the prevention of ABAP-induced DCFH oxidation in HepG2 cells by broccoli extracts was not correlated to ORAC, indicating that the chemical assay may not be a good measure of antioxidant activity in biological models (Eberhardt, M. V., et al (2005), supra).

### J. Summary.

The CAA assay reported here is a great improvement over the “test tube” chemical methods used to evaluate the efficacy of pure phytochemical compounds, plant extracts, and dietary supplements. It is an assay for screening antioxidants that considers cellular uptake, distribution, and efficiency of protection against peroxyl radicals under physiological conditions. The CAA assay presented here answers the demand for the next step forward from chemistry assays to assess the potential bioactivity of antioxidants.

### Example 2

### Cellular Antioxidant Activity of Common Fruits

Example 2 shows that the methods described herein are useful for evaluating the antioxidant activity of common fruits consumed in the United States.

### A. Background

Free radicals are reactive molecules with unpaired electrons that are able to exist independently. Endogenous metabolic processes, especially in chronic inflammations, are important sources of free radicals (Liu, R. H. et al, (1995) Mutat. Res. 339(2):73-89), which can react with and damage all types of biomolecules, lipids, proteins, carbohydrates, and DNA (Ames, B. N. and Gold, L. S. (1991) Mutat. Res. 250(1-2):3-16). If damaged DNA is left unrepaired, and the mutated cell gains the ability to survive and divide aberrantly, it may become cancerous. Thus, an increase in antioxidants, which can scavenge free radicals, may be a strategy to prevent cancer cell initiation, an important beginning stage of carcinogenesis.

Doll and Peto (Doll, R. and Peto, R. (1981) J. Natl. Cancer Inst. 66(6):1191-1308) proposed that diet is responsible for about one-third of cancer incidence. Several associations have been made between fruit and vegetable intake and a reduced risk of cancer (Steinmetz, K. A. and Potter, J. D. (1996) J. Am. Diet. Assoc. 96(10):1027-1039; Block, G, et al (1992) Nutr. Cancer 18(1):1-29; Cohen, J. H. et al (2000) J. Natl. Cancer Inst. 92(1):61-68; Chan, J. M. et al (2005) Cancer Epidemiol. Biomarkers PreV. 14(9):2093-2097; Feskanich, D. et al (2000) J. Natl. Cancer Inst. 92(22):1812-1823; Michels, K. B. et al (2006) Cancer Res. 66(7):3942-3953; Lunet, N. et al (2005) Nutr. Cancer 53(1):1-10)

Higher fruit intake in childhood has also been related to lower adult cancer risk (Maynard, M. et al (2003) J. Epidemiol. Community Health 57(3):218-225). Fruits are rich in bioactive phenolic compounds such as flavonoids, phenolic acids, stilbenes, coumarins, and tannins. The combined phytochemicals in plant foods have a variety of mechanisms of action, including effects on antioxidant activity and free radicals, cell cycle, oncogene and tumor suppressor gene expression, apoptosis, detoxifying enzyme activity, immunity, metabolism, and infection (Liu, R. H. (2004) J. Nutr. 134(12):34795-34855). In a study that evaluated the effect of antioxidant activity on gastric cancer risk, antioxidant activity obtained from fruit and vegetable consumption was inversely associated with risk of gastric cancer (Serafini, M. et al (2002) Gastroenterology 123(4):985-991). The latest report by the Economic Research Service states that U.S. fruit and vegetable consumption increased between 1970 and 2005, but that Americans are still not eating enough of these plant foods for optimum health (Wells, H. F. and Buzby, J. C. (2008) Economic Research Bulletin 3. Dietary Assessment of Major Trends in U.S. Food Consumption, 1970-2005; U.S. Department of Agriculture: Washington, D.C.). The 2005 Dietary Guidelines for Americans (U.S. Department of Agriculture, U.S. Department of Health and Human Services. Dietary Guidelines for Americans, 2005, 6th ed.; U.S. Government Printing Office: Washington, D.C.) recommend each person eats 2 cups (four servings) of fruit and 2.5 cups (five servings) of vegetables, based on a 2000 kcal diet, but the study found that in 2005 the average intake of fruits was only 0.9 cups and vegetable intake was 1.7 cups per day (Wells, H. F. and Buzby, J. C. (2008), supra).

Due to the potential of antioxidants to decrease the risk of developing cancer and other chronic diseases, it is important to be able to measure antioxidant activity using biologically relevant assays such as cellular antioxidant activity (CAA) assay described herein. The antioxidant activity of fruits has been surveyed using the oxygen radical absorbance capacity (ORAC) assay (Wang, H. et al (1996) J. Agric. Food Chem. 44:701-705; Proteggente, A. R. et al (2002) Free Radical Res. 36(2):217-233), inhibition of cupric ion-induced oxidation of lipoproteins (Vinson, J. A.; et al (2001) J. Agric. Food Chem. 49:5315-5321), total oxyradical scavenging capacity (TOSC) assay (Sun, J. et al (2002) J. Agric. Food Chem. 50:7449-7454), ferric reducing/antioxidant power (FRAP) assay (Proteggente, A. R. et al (2002), supra; Halvorsen, B. L. et al (2002) J. Nutr. 132(3):461-471; Pellegrini, N. et al. (2003) J. Nutr. 133(9): 2812-2819), Trolox equivalent antioxidant capacity (TEAC) assay (Proteggente, A. R. et al (2002), supra, Pellegrini, N. et al. (2003), supra), and total radical-trapping antioxidant parameter (TRAP) assay (Pellegrini, N. et al. (2003), supra). The antioxidant activities of a wide variety of fruits using a cell-based model are described herein below.

The objective of this study was to determine the cellular antioxidant activity of 25 commonly consumed fruits using the CAA assay, as described herein. The total phenolic contents and ORAC values of the fruits were also measured to determine if they could be used to predict CAA values. The antioxidant quality of the fruits in the CAA assay and their individual contributions to the antioxidant activity of fruits in the American diet were calculated.

### B. Materials and Methods

### Chemicals

2′,7′-Dichlorofluorescin diacetate (DCFH-DA), fluorescein disodium salt, 6-hydroxy-2,5,7,8-tetramethylchoman-2-carboxylic acid (Trolox), Folin-Ciocalteu reagent, and quercetin dehydrate were purchased from Sigma-Aldrich, Inc. (St. Louis, Mo.). Dimethyl sulfoxide was obtained from Fisher Scientific (Pittsburgh, Pa.), and 2,2′-azobis (2-amidinopropane) dihydrochloride (ABAP) was purchased from Wako Chemicals USA, Inc. (Richmond, Va.). Sodium carbonate, methanol, acetone, and potassium phosphate were bought from Mallinckrodt Baker, Inc. (Phillipsburg, N.J.), and gallic acid was from ICN Biomedical Inc. (Costa Mesa, Calif.). HepG2 liver cancer cells were obtained from the American Type Culture Collection (ATCC) (Rockville, Md.). Williams' Medium E (WME) and Hanks' Balanced Salt Solution (HBSS) were purchased from Gibco Life Technologies (Grand Island, N.Y.). Fetal bovine serum (FBS) was obtained from Atlanta Biologicals (Lawrenceville, Ga.).

### Preparation of Fruit Extracts

Apples were purchased from Cornell Orchards (Cornell University, Ithaca, N.Y.), and wild blueberries were obtained from the Wild Blueberry Association of North America (Damariscotta, Me.). All other fruits were purchased at a local supermarket (Ithaca, N.Y.). Fruit phytochemical extracts were prepared from the edible portions of fruits using a modified method, as reported previously (Sun, J. et al (2002), supra). Briefly, in triplicate, fresh fruit samples were blended for 5 min in chilled 80% acetone (1:2, w/v) using a Waring blender. Samples were then homogenized with a Polytron homogenizer for 3 min. The homogenates were filtered through Whatman no. 1 paper, and the filtrates were evaporated to dryness under vacuum at 45° C. The samples were reconstituted in 70% methanol and stored at −40° C. Before use, the methanol was evaporated under a stream of nitrogen, and the extracts were reconstituted in water.

### Preparation of Solutions

A 200 mM stock solution of DCFH-DA in methanol was prepared, aliquoted, and stored at −20° C. A 200 mM ABAP stock solution in water was prepared, and aliquots were stored at −40° C. Quercetin solutions were prepared in dimethyl sulfoxide before further dilution in treatment medium (WME with 2 mM L-glutamine and 10 mM Hepes).

### Cell Culture

HepG2 cells were cultured as described herein in Example 1.

### Cytotoxicity

The cytotoxicity of fruits toward HepG2 cells was measured, as described previously (Wolfe, K. L and Liu, R. H. (2007), supra; Yoon, H. and Liu, R. H. (2007) J. Agric. Food Chem. 55:3167-3176). The median cytotoxic concentration (CC50) was calculated for each fruit.

### Cellular Antioxidant Activity (CAA) of Fruit Extracts

The CAA assay protocol was described previously (Wolfe, K. L and Liu, R. H. (2007), supra). Briefly, HepG2 cells were seeded at a density of 6×10⁴/well on a 96-well microplate in 100 μL of growth medium/well. Twenty-four hours after seeding, the growth medium was removed, and the wells were washed with PBS. Wells were treated in triplicate for 1 h with 100 μL of treatment medium containing tested fruit extracts plus 25 μM DCFH-DA. When a PBS wash was utilized, wells were washed with 100 μL of PBS. Then 600 μM ABAP was applied to the cells in 100 μL of HBSS, and the 96-well microplate was placed into a Fluoroskan Ascent FL plate reader (ThermoLabsystems, Franklin, Mass.) at 37° C. Emission at 538 nm was measured after excitation at 485 nm every 5 min for 1 h.

### Quantification of CAA

After blank subtraction and subtraction of initial fluorescence values, the area under the curve for fluorescence versus time was integrated to calculate the CAA value at each concentration of fruit as (Wolfe, K. L and Liu, R. H. (2007), supra)

CAA unit=1−(∫SA/∫CA)

where ∫SA is the integrated area under the sample fluorescence versus time curve and ∫CA is the integrated area from the control curve. The median effective dose (EC₅₀) was determined for the fruits from the median effect plot of log(fa/fu) versus log(dose), where fa is the fraction affected (CAA unit) and fu is the fraction unaffected (1-CAA unit) by the treatment. The EC₅₀ values were stated as mean±SD for triplicate sets of data obtained from the same experiment. EC₅₀ values were converted to CAA values, expressed as micromoles of quercetin equivalents (QE) per 100 g of fruit, using the mean EC50 value for quercetin from five separate experiments.

### Determination of Total Phenolic Content

The total phenolic contents of the fruits were measured using a modified colorimetric Folin-Ciocalteu method (Wolfe, K. L and Liu, R. H. (2007), supra, Singleton, V. L. et al (1999) In Methods in Enzymology; Academic Press: New York; Vol. 299, pp 152-178). Volumes of 0.5 mL of deionized water and 0.125 mL of diluted fruit extracts were added to a test tube. Folin-Ciocalteu reagent (0.125 mL) was added to the solution and allowed to react for 6 min. Then, 1.25 mL of 7% sodium carbonate solution was aliquoted into the test tubes, and the mixture was diluted to 3 mL with deionized water. The color was developed for 90 min, and the absorbance was read at 760 nm using a MRX II Dynex spectrophotometer (Dynex Technologies, Inc., Chantilly, Va.). The measurement was compared to a standard curve of gallic acid concentrations and expressed as milligrams of gallic acid equivalents (GAE) per 100 g of fresh fruit±SD for triplicate fruit extracts.

### Measurement of Oxygen Radical Scavenging Capacity (ORAC)

The peroxyl radical scavenging efficacy of selected fruits was measured using the ORAC assay (Prior, R. L., et al (2003) J. Agric. Food Chem. 51:3273-3279). Briefly, 20 μL of blank, Trolox standard, or fruit extracts in 75 mM potassium phosphate buffer, pH 7.4 (working buffer), was added to triplicate wells in a black, clear-bottom, 96-well microplate. The triplicate samples were distributed throughout the microplate and were not placed side-by-side, to avoid any effects on readings due to location. In addition, no outside wells were used, as use of those wells results in greater variation. A volume of 200 μL of 0.96 μM fluorescein in working buffer was added to each well and incubated at 37° C. for 20 min, with intermittent shaking, before the addition of 20 μL of freshly prepared 119 mM ABAP in working buffer using a 12-channel pipetter. The microplate was immediately inserted into a Fluoroskan Ascent FL plate reader (ThermoLabsystems) at 37° C. The decay of fluorescence at 538 nm was measured with excitation at 485 nm every 4.5 min for 2.5 h. The areas under the fluorescence versus time curve for the samples minus the area under the curve for the blank were calculated and compared to a standard curve of the areas under the curve for 6.25, 12.5, 25, and 50 μM Trolox standards minus the area under the curve for blank. ORAC values were expressed as mean micromoles of Trolox equivalents (TE) per 100 g of fruit±SD for triplicate data from one experiment.

### Statistical Analyses

All results are presented as mean±SD, and statistical analyses were performed using Minitab 15 (Minitab Inc., State College, Pa.). Differences between means were detected by ANOVA, followed by multiple comparisons using Fisher's least significant difference test. ANOVA was performed on log-transformed total phenolic, ORAC, and CAA values because the assumptions of normally distributed residuals and equal variances were not met by the untransformed data. Correlations were determined using linear regression on log-transformed data. Differences between mean EC₅₀ values for CAA, comparing the results from the no PBS wash and PBS wash protocols, were evaluated using a two-tailed paired Student's t test. Determination of differences between cellular antioxidant quality for each fruit was performed using a paired Student's t test on normalized (antioxidant quality−mean antioxidant quality)/standard deviation for antioxidant qualities in protocol) values for those fruits with activity in both the no PBS wash and PBS wash protocols. Normalization was necessary because the two values could not be compared directly. For those fruits with no activity in the PBS wash protocol, the difference between the cellular antioxidant quality in the no PBS wash protocol and zero was determined using a one-way Student's t test. Interaction between the fruit and the protocol in cellular antioxidant quality was assessed by two-way ANOVA of the normalized antioxidant qualities. Results were considered to be significant when p value <0.05.

### C. Results

### Total Phenolic Content

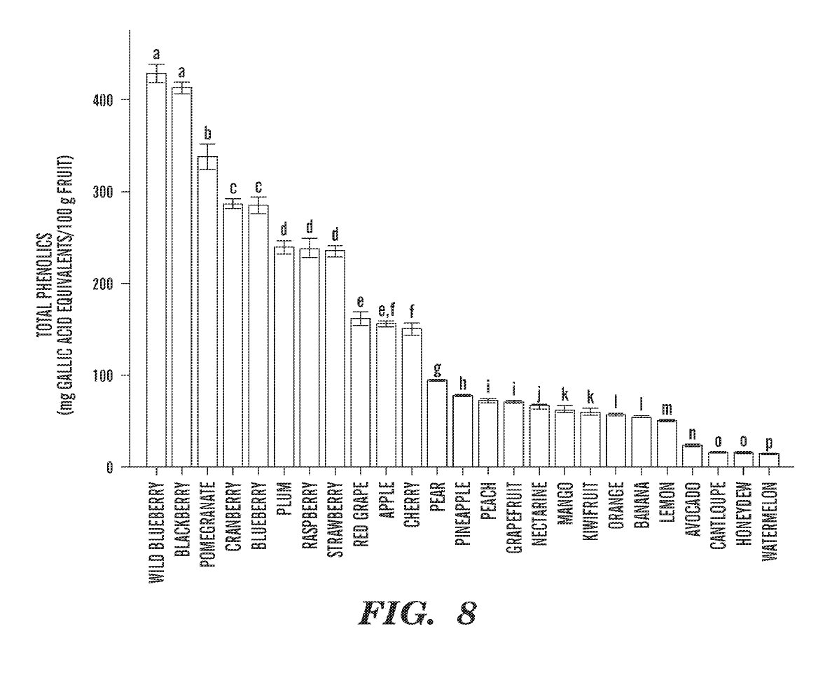

The total phenolic content of selected fruits (FIG. 8) was determined from their extracts using the Folin-Ciocalteu method. Among the fruits, wild blueberry and blackberry had the highest total phenolic contents (429±10 and 412±6 mg of GAE/100 g, respectively), followed by pomegranate (338±14 mg of GAE/100 g); cranberry and blueberry (287±5 and 285±9 mg of GAE/100 g, respectively); plum, raspberry, and strawberry (239±7, 239±10, and 235±6 mg of GAE/100 g, respectively); and red grape and apple (161±7 and 156±3 mg of GAE/100 g, respectively). The total phenolic content of cherry (151±6 mg of GAE/100 g) was not significantly different from that of apple. The remaining fruits in order of total phenolic content were pear (94.8±0.7 mg GAE/100 g)>pineapple (78.1±0.8 mg of GAE/100 g)>peach (73.1±2.4 mg of GAE/100 g)=grapefruit (71.0±1.3 mg of GAE/100 g)>nectarine (66.3±2.1 mg of GAE/100 g)>mango (62.6±4.2 mg of GAE/100 g)=kiwifruit (60.4±3.3 mg of GAE/100 g)>orange (56.9±0.8 mg of GAE/100 g)=banana (54.8±1.3 mg of GAE/100 g)>lemon (50.8±0.9 mg of GAE/100 g)>avocado (23.9±0.7 mg of GAE/100 g)>cantaloupe (16.0±0.4 mg of GAE/100 g)=honeydew (15.5±0.9 mg of GAE/100 g)>watermelon (14.1±0.3 mg of GAE/100 g).

### ORAC

The antioxidant activities of the selected fruits (FIG. 9) were evaluated using the ORAC assay. Wild blueberry, cranberry, and strawberry had the greatest peroxyl radical scavenging ability in this method, with ORAC values of 9621±1080, 8394±1405, and 8348±888 μmol of TE/100 g of fruit, respectively. The next highest ORAC values were obtained from blackberry (6221±43 μmol of TE/100 g), cherry (5945±978 μmol of TE/100 g), plum (5661±440 μmol of TE/100 g), and raspberry (5292±877 μmol of TE/100 g of fruit), which were similar (p>0.05). The other fruits had ORAC values of 4826±649 μmol of TE/100 g (blueberry), 4592±201 μmol of TE/100 g (apple), 4479±378 μmol of TE/100 g (pomegranate), 2887±717 μmol of TE/100 g (orange), 2605±487 μmol of TE/100 g (red grape), 2235±278 μmol of TE/100 g (peach), 1848±186 μmol of TE/100 g (lemon), 1759±136 μmol of TE/100 g (pear), 1640±299 μmol of TE/100 g (grapefruit), 1586±51 μmol of TE/100 g (nectarine), 1385±11 μmol of TE/100 g (watermelon), 1343±158 μmol of TE/100 g (avocado), 1262±132 μmol of TE/100 g (kiwifruit), 1164±155 μmol of TE/100 g (mango), 1055±84 μmol of TE/100 g (pineapple), and 565±18 μmol of TE/100 g (banana). Cantaloupe and honeydew melon had the lowest antioxidant capacity in the ORAC assay (237±22 and 274±31 μmol of TE/100 g of fruit, respectively). With a few exceptions, the ORAC data described herein in this example for fruits correspond well with those reported by the U.S. Department of Agriculture (U.S. Department of Agriculture, Agriculture Research Service, Oxygen Radical Absorbance Capacity (ORAC) of Selected Foods (2007). Nutrient Data Laboratory Website, available on the world wide web at .ars.usda.gov/nutrientdata): only strawberry, cherry, red grape, and watermelon tested in this study had higher ORAC values.

### CAA

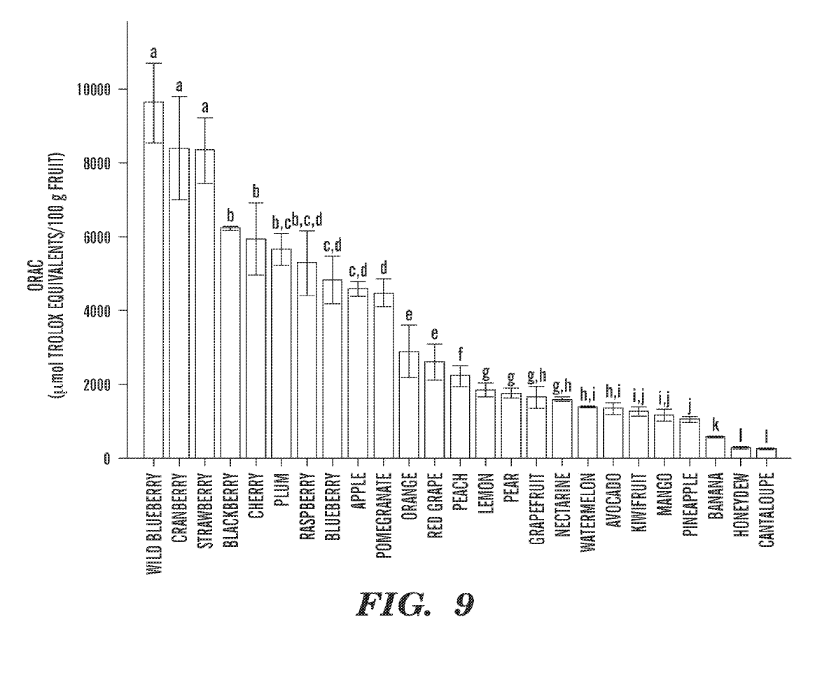

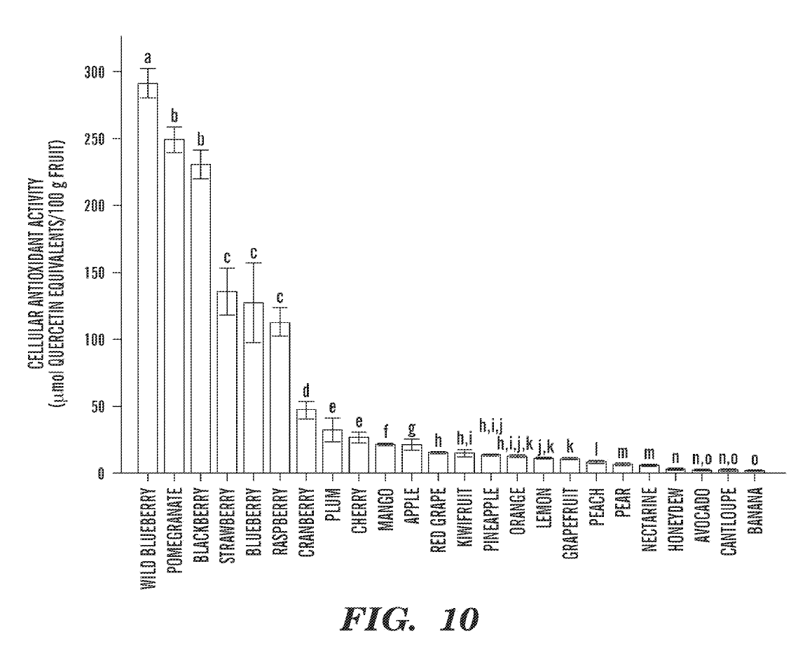

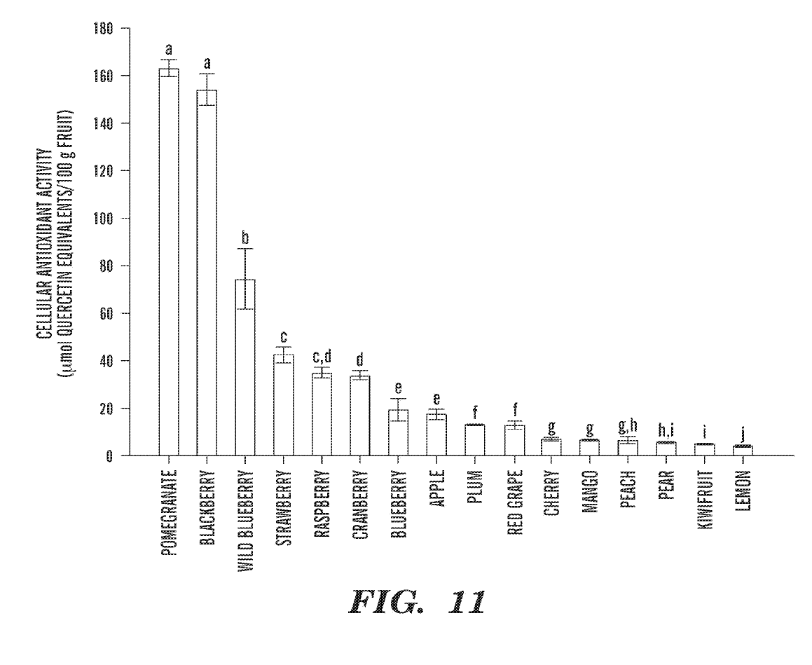

The cellular antioxidant activities of selected fruits were measured using the CAA assay. The EC₅₀ and CAA values for the fruits, along with their median cytotoxicity doses, are listed in Table 5. The cellular antioxidant activities were measured using two protocols, as described previously (Wolfe, K. L and Liu, R. H. (2007), supra): in the PBS wash protocol, the HepG2 cells were washed with PBS between fruit extract and ABAP treatments; in the no PBS wash protocol, the cells were not washed between treatments. Both protocols were used because the difference between them provides insight into how the antioxidants interact with the cells. In most cases, the EC₅₀ values were significantly lower, and efficacy was higher, in the no PBS wash protocol compared to the PBS wash protocol for each fruit. However, there were no significant differences between the EC₅₀ values obtained from the two protocols for pomegranate, blackberry, cranberry, apple, red grape, peach, and pear. The CAA values for the fruits in the no PBS wash protocol are shown in FIG. 10 and Table 5. Wild blueberry had the highest CAA value (292±11 μmol of QE/100 g of fruit), followed by pomegranate and blackberry, which had similar CAA values (p>0.05). Strawberry, blueberry, and raspberry were next and were not significantly different from each other (p>0.05). These were followed by cranberry, plum, cherry, mango, apple, red grape, kiwifruit, pineapple, orange, lemon, grapefruit, peach, pear, nectarine, and honeydew. Banana, cantaloupe, and avocado had the lowest CAA values, among the fruits. Watermelon was the only fruit tested that did not have quantifiable activity. In the PBS wash protocol, pomegranate and blackberry had the greatest cellular antioxidant activity, with CAA values of 163±4 and 154±7 μmol of QE/100 g of fruit, respectively (FIG. 11; Table 5). Wild blueberry ranked second for efficacy, and strawberry and raspberry were third. In declining order of cellular antioxidant activity, the remaining fruits were cranberry, blueberry, apple, plum, red grape, cherry, mango, peach, pear, and kiwifruit. Lemon had the lowest CAA value (3.68±0.211 μmol of QE/100 g of fruit). Pineapple, orange, peach, nectarine, honeydew, avocado, cantaloupe, banana, and watermelon all had very low activities that could not be quantified in the PBS wash protocol.

### Correlation Analyses

Using regression analyses, the relationships between total phenolic content, ORAC value, and CAA value for the fruits were determined. Total phenolics were significantly correlated to ORAC values (R²=0.761, p<0.05) and CAA values from the no PBS wash protocol (R²=0.811, p<0.05) and PBS wash protocols (R²=0.793, p<0.05). ORAC values for fruits were also significantly positively related to CAA values, although the correlation coefficients were lower (R²=0.678, p<0.05 for no PBS wash protocol; R²=0.522, p<0.05 for PBS wash protocol).

### Cellular Antioxidant Quality

The cellular antioxidant quality of the phytochemical extracts was determined for the fruits from their CAA values and total phenolic contents (Table 6). This is a measurement of the cellular antioxidant activity, in quercetin equivalents, per 100 μmol of phenolic compounds present in the fruit and was described previously (Wolfe, K. L and Liu, R. H. (2007), supra). The cellular antioxidant quality from the fruits in the no PBS protocol ranged from 1.0 (0.1 μmol of QE/100 μmol of phenolics (banana) to 12.6±0.5 μmol of QE/100 μmol of phenolics (pomegranate). Pomegranate was followed by wild blueberry, strawberry, blackberry, raspberry, blueberry, kiwifruit, honeydew, mango, lemon, orange, cantaloupe, pineapple, cherry, cranberry, grapefruit, avocado, apple, plum, peach, nectarine, red grape, and pear. The range of antioxidant qualities in the PBS wash protocol was from 0.8±0.1 μmol of QE/100 μmol of phenolics (cherry) to 8.2±0.2 μmol of QE/100 μmol of phenolics (pomegranate). The remaining fruits, in order of highest to lowest cellular antioxidant quality, were blackberry, strawberry, wild blueberry, raspberry, cranberry, apple, mango, peach, red grape, kiwifruit, lemon, blueberry, pear, and plum. There was a significant interaction between the protocol and fruits (p<0.05). Because the antioxidant qualities of each fruit obtained from the no PBS wash and PBS wash protocols could not be compared directly, the values were normalized. After normalization, it was found that, relative to the other fruits, the antioxidant qualities of pomegranate, blackberry, cranberry, apple, peach, red grape, and pear were significantly lower in the no PBS wash protocol than in the PBS protocol, whereas the antioxidant qualities of wild blueberry, raspberry, and blueberry were higher in the no PBS wash protocol (p<0.05). There was no difference between normalized antioxidant qualities from the two protocols for strawberry, kiwifruit, honeydew, mango, lemon, cantaloupe, pineapple, cherry, plum, and nectarine (p>0.05).

### Contribution of Fruits to Dietary Phenolics and Cellular Antioxidant Activity

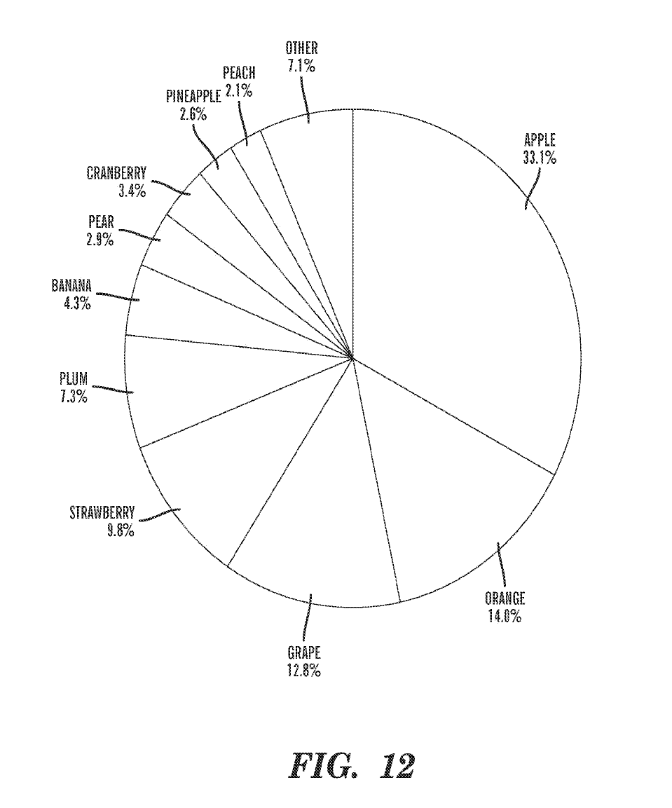

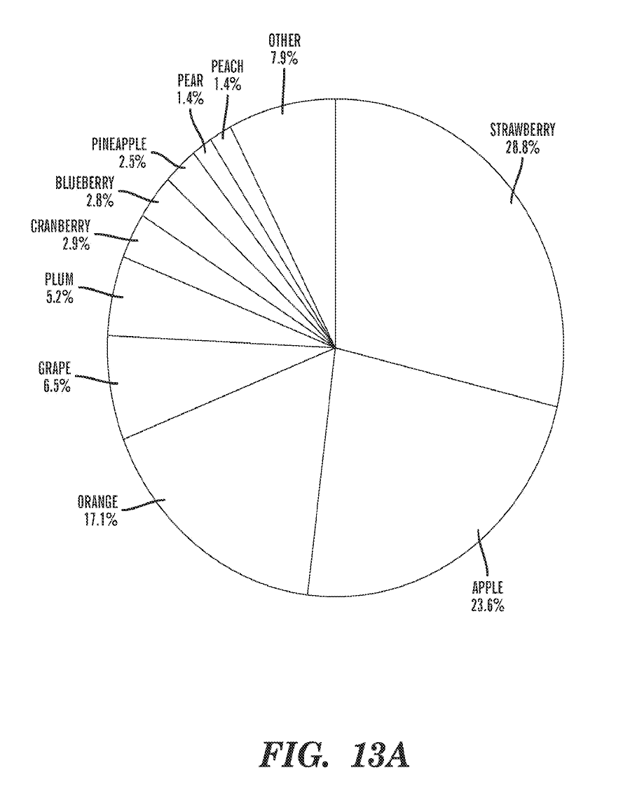

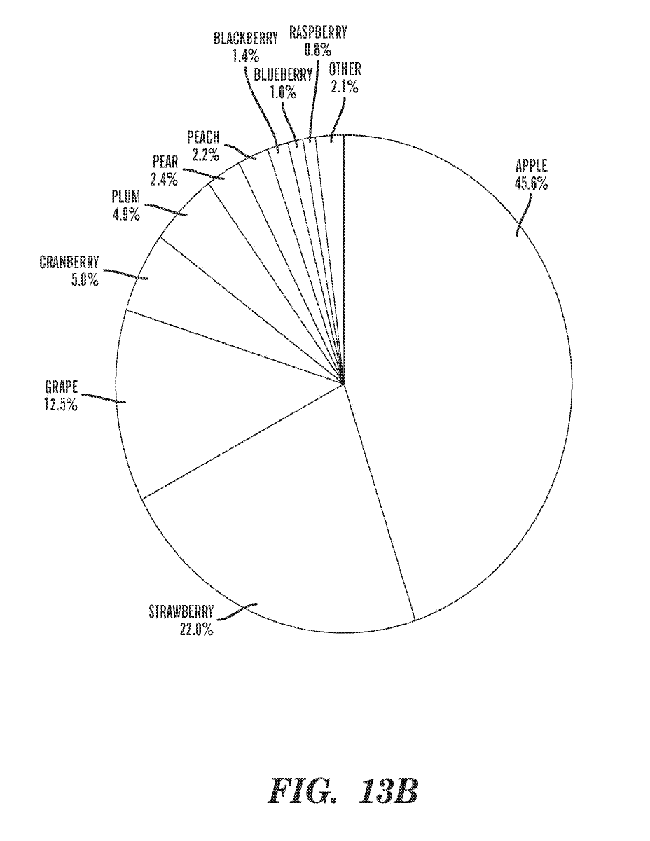

The contribution of the selected fruits to the total phenolics and CAA in the United States from all fruits in the American diet was calculated from consumption data from the U.S. Department of Agriculture Food Availability (Per Capita) Data for 2005 (U.S. Department of Agriculture, Economic Research Service. (2007) ERS Loss-Adjusted Food Availability Data, available on the world wide web at ers.usda.gov/Data/FoodConsumption/FoodAvail-Index.htm). Loss-adjusted food availability data for fresh, canned, frozen, dried, and juice were used, which are adjusted for nonedible fruit parts and losses due to waste, spoilage, and other factors. The top 10 phenolic contributors expressed as a percentage of the total phenolic contribution from fruits in the American diet are shown in FIG. 12. Apples were the largest supplier of fruit phenolics to the population (33.1%), followed by orange (14.0%), grape (12.8%), and strawberry (9.8%). Plum, banana, pear, cranberry, pineapple, and peach rounded out the top 10. The contributions of the selected fruits to cellular antioxidant activity, as calculated from the no PBS wash protocol results (FIG. 13A), were similar to the phenolic contributions, with strawberry (28.8%), apple (23.6%), orange (17.1%), and grape (6.5%) providing the most CAA. Plum, cranberry, blueberry, pineapple, pear, and peach were also top 10 contributors. From the PBS wash protocol data (FIG. 13B), the most cellular antioxidant activity for fruits, by far, was supplied by apple at 45.6%, followed by strawberry (22.0%) and grape (12.5%). Most of the remaining activity from fruits was contributed by cranberry, plum, pear, peach, blackberry, blueberry, and raspberry.

The CAA assay described herein is a valuable new tool for measuring the antioxidant activity of antioxidants, dietary supplements, and foods in cell culture (Wolfe, K. L and Liu, R. H. (2007), supra). It is an improvement over the traditional chemistry antioxidant activity assays because it takes into account some aspects of cell uptake, metabolism, and distribution of bioactive compounds, which are important modulators of bioactivity (Spencer, J. P. E.; et al (2004) Arch. Biochem. Biophys. 423(1):148-161), so it may better predict antioxidant behavior in biological systems. The assay utilizes HepG2 cells because they yield consistent results with lower coefficient of variation. Results obtained from other cell lines, including intestinal Caco-2 cells and RAW 264.7 cells, were similar to those found using HepG2 cells, but with much higher variation (data not shown). In addition, HepG2 cells are a better model choice to address metabolism issues.

In general, the CAA values of the berries (wild blueberry, blackberry, strawberry, blueberry, raspberry, and cranberry) tended to be the highest (FIGS. 10 and 11). They also had among the most total phenolics (FIG. 8) and the top ORAC values (FIG. 9). The high antioxidant efficacy of berries in the CAA and ORAC assays is in agreement with that measured in other antioxidant activity assays (Vinson, J. A.; et al (2001), supra; Pellegrini, N. et al. (2003), supra). Berries tend to be rich in anthocyanins, and fruits rich in those flavonoids have high activity in the TEAC, FRAP, and ORAC assays (Proteggente, A. R. et al (2002), supra). Pomegranate had very high activity in the CAA assay, ranking first in the PBS wash protocol and second in the no PBS protocol. Pomegranate also had the highest activity among the fruits tested by Halvorsen et al. (Halvorsen, B. L. et al (2002), supra) using the FRAP assay. Despite having a very high total phenolic content, pomegranate did not rank highly in the ORAC assay. The melons had the lowest activities of all the fruits in the CAA assay. They had such low effectiveness using the PBS wash protocol that CAA values could not be quantified. The melons also had low total phenolic contents and low ORAC values. Melons ranked low among fruits in antioxidant activity in other studies (Vinson, J. A.; et al (2001), supra; Pellegrini, N. et al. (2003), supra; Halvorsen, B. L. et al (2002), supra), as well.

The CAA values for fruits were significantly positively related to total phenolic content when log-transformed data were analyzed (p<0.05). The correlation coefficients for CAA values and total phenolics were R²=0.811 for the no PBS wash protocol and R²=0.793 for the PBS wash protocol. The log-transformed CAA and ORAC values were also significantly correlated (R²=0.678 for the no PBS wash protocol; R²=0.522 for the PBS wash protocol, p <0.05). This is in contrast to a study involving broccoli extracts, in which prevention of DCFH oxidation in HepG2 cells by broccoli extracts was not correlated to ORAC or total phenolics (Eberhardt, M. V. et al (2005) J. Agric. Food Chem. 53:7421-7431). From the results of our study, total phenolic content is likely a better predictor for the cellular antioxidant activity of fruits than ORAC value, despite the commonality of measuring peroxyl radical scavenging abilities in both of the antioxidant activity assays.

The EC₅₀ values for CAA were similar in the no PBS wash and PBS protocols for pomegranate, blackberry, cranberry, apple, red grape, peach and pear, whereas the rest of the fruits showed lower activities and higher EC₅₀ values in the PBS wash protocol. This is likely a reflection of the type and location of the fruit antioxidants in the HepG2 cells. The differences in solubility, molecular size, and polarity of the wide variety of compounds present in fruits and vegetables give each of them unique bioavailability and distribution at the cellular, organ, and tissue levels, allowing for bioactivity at many sites (Liu, R. H. (2004), supra). Some phenolics, such as quercetin, epigallocatechin gallate, and luteolin, showed similar cellular antioxidant activity in both the no PBS wash protocol and the PBS wash protocol (Wolfe, K. L. and Liu, R. H. (2007), supra). Others, such as gallic acid, caffeic acid, and catechin, displayed a dramatic decrease in activity when a PBS wash was done between phytochemical and oxidant (ABAP) treatments, compared when no PBS was performed (Wolfe, K. L. and Liu, R. H. (2007), supra). Those phenolics that are better absorbed by the HepG2 cells or tightly bound to the cell membrane are more likely to be present to exert their radical scavenging activities after the cells are washed in the PBS wash protocol than those that are poorly absorbed or only loosely associated with the cell membrane and washed away easily. Thus, the difference in EC₅₀ values (and CAA values) between the two protocols is likely a good indicator of the extent of uptake and cell membrane association of the antioxidant compounds present in the fruit extracts. Cellular antioxidant quality is a measure of the cellular antioxidant activity provided by 100 μmol of phenolics found in the fruit, so it gives a relative potency of the antioxidants present. An index of antioxidant quality, expressed as phenolic content/IC₅₀ for inhibition of lipoprotein oxidation, has also been used by Vinson et al. (Vinson, J. A. et al (2001), supra) to assess fruits. Pomegranate had the highest antioxidant quality in both the PBS wash and no PBS wash protocols (Table 6). Wild blueberry, strawberry, blackberry, and raspberry also ranked highly in both protocols. For all fruits in our study, the antioxidant quality was lower from the PBS wash protocol than from the no PBS protocol, even for those fruits with similar EC₅₀ values in both protocols (Tables 5 and 6). This is due to the quercetin standard's aberrant behavior of having higher activity, and a lower EC₅₀ value, in the PBS wash protocol than in the no PBS wash protocol, which was also seen previously (Wolfe, K. L. and Liu, R. H. (2007), supra). Because the cellular antioxidant quality values for each fruit in the two protocols were not comparable, the values were normalized. Wild blueberry, raspberry, and blueberry had lower cellular antioxidant quality in the PBS wash protocol than in the no PBS protocol, indicating that, relative to the other fruits, the phenolic antioxidants in these fruits are taken up less well by the cells or bound less tightly to the cell membrane. The normalized antioxidant qualities of pomegranate, blackberry, cranberry, apple, peach, red grape, and pear were higher in the PBS wash protocol, suggesting their phenolics were more closely associated with the cells than those from the other fruits. The contribution of total phenolics from fruits in the American diet was estimated from our total phenolic measurements and per capita loss-adjusted food availability data for the United States (U.S. Department of Agriculture, Economic Research Service. (2007), supra). Apple was the largest contributor to total phenolics (FIG. 12) of all fruits consumed by Americans. In comparison to the other fruits examined, apple had medium phenolic content, but the per capita consumption of apples is high (U.S. Department of Agriculture, Economic Research Service. (2007), supra). Other substantial contributors to phenolic intake were orange, grape, strawberry, and plum. The percent contribution of phenolics from orange and banana were 14.0 and 4.3%, respectively, because of high consumption, despite their comparatively low total phenolic contents (FIG. 8). Our ranking of phenolic contribution from fruits differed greatly from that published in 2001 by Vinson et al. (Vinson, J. A. et al (2001), supra), who placed banana at the top and included watermelon and cantaloupe in the top six. The differences in rankings can be explained by three major factors: juice consumption data were included in our study and not in the analysis by Vinson et al.; phenolics were measured using a catechin standard curve in the earlier study by Vinson et al., instead of the gallic acid standard curve we used; and consumption patterns may have changed. Contribution of CAA activities from fruits in the American diet was also discussed. Strawberry, apple, orange, and grape were the top providers of CAA from the no PBS wash protocol (FIG. 13A), whereas apple, strawberry, grape, and cranberry were the highest contributors from the PBS wash protocol (FIG. 13B). Strawberry ranked well because of its high activity, even though its consumption is not great. Banana did not even place in the top 10 contributors of fruit cellular antioxidant activity in the no PBS wash protocol due to its low CAA value. Orange and banana did not have any activity in the PBS wash protocol, so despite the high intake of oranges and bananas in the United States, they did not supply any PBS wash CAA to the population. Small increases in the consumption of berries, such as blueberry, blackberry, cranberry, and raspberry, would have a large impact on their percent contributions figures because of their very high phenolic content and cellular antioxidant activity.

### Example 3

### Structure-Activity Relationships of Flavonoids in the Cellular Antioxidant Activity Assay

### A. Background

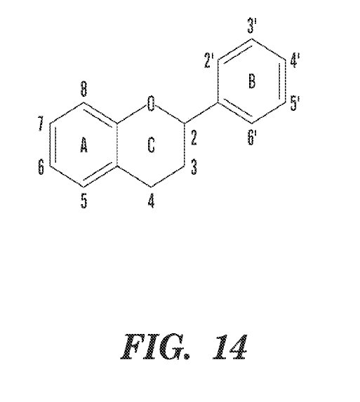

Cancer is the second leading cause of death in the United States (Minino, A et al (2006) In National Vital Statistics Reports; National Center for Health Statistics: Hyattsville, Md., Vol. 54). Cancer is a disease in which abnormally high proliferation of mutated cells occurs. Oxidative stress may be the most important factor causing oxidative DNA damage that can eventually lead to mutations if left unrepaired (Ames, B. N and Gold, L. S. (1991) Mutat. Res. 250(1-2):3-16). Consumption of fruits and vegetables has been linked to reduced risk of cancer in several epidemiological studies (Steinmetz, K. A. and Potter, J. D. (1996), supra; Block, G. et al (1992) Nutr. Cancer 18(1):1-29). The dietary phytochemicals in fruits and vegetables are likely responsible for decreased cancer risk by reducing oxidative stress and modulating signal transduction pathways involved in cell proliferation and survival (Williams, R. J. et al. (2004) Free Radical Biol. Med. 36(7):838-849; Liu, R. H (2004) J. Nutr. 34(12):34795-34855). The flavonoids are a class of widely distributed phytochemicals with antioxidant and biological activity. They have structures consisting of two aromatic rings linked by three carbons in an oxygenated heterocycle (FIG. 14). Differences in the structure of the heterocycle, or C-ring, classify them as flavonols, flavones, flavanols (catechins), flavanones, anthocyanidins, or isoflavonoids (isoflavones) (Liu, R. H. (2004), supra).

Flavonols are characterized by a 2,3-double bond, a 4-keto group, and a 3-hydroxyl group in the C-ring. Flavones lack the 3-hydroxyl moiety, and flavanones have a saturated C-ring. The 2,3-double bond and 4-keto group are absent from flavanols or catechins. The B-ring of isoflavones is linked to C-3 of the C-ring, instead of C-2, as it is for the other flavonoid subclasses. Flavonoids, as constituents of plant foods, have been implicated in the reduction of cancer risk. In the Zutphen Elderly Study, flavonoid intake from fruits and vegetables was inversely associated with all-cause cancer risk and cancer of the alimentary and respiratory tract (Hertog, M. G. L. et al (1994) Nutr. Cancer 22(2):175-184). Lung cancer risk has also been inversely associated with flavonoid (Knekt, P. et al (1997) Am. J. Epidemiol. 146(3):223-230) and quercetin intake (Le Marchand, L. et al (2000) J. Natl. Cancer Inst 92(2):154-160). Although not definitive, many other epidemiological studies have shown a trend for decreased cancer risk with higher flavonoid consumption, and these studies have been reviewed recently (Neuhouser, M. L. et al (2004) Nutr. Cancer 50(1):1-7; Graf, B. A. et al (2005) J. Med. Food 8(3); 281-290). Concepts related to the presence of antioxidants in foods and their potential health benefits to humans are becoming recognized (Liu, R. H., (2004), supra).

Described herein are methods for measuring antioxidant activity that have more biological relevance than simple chemical methods that measure antioxidant activity in controlled systems, but are not reflective of biological activity because they do not account for cell uptake, partitioning of antioxidants between aqueous and lipid phases, or phase I and phase II metabolism. The CAA assay measures the inhibition of peroxyl radical-induced oxidation of dichlorofluorescin by antioxidants in cell culture.

It was proposed by Bors et al. (Bors, W. et al (1990) Methods Enzymol. 186:343-355) that three structural moieties are important for antioxidant and radical-scavenging activity by flavonoids: (1) an o-dihydroxyl group in the B-ring; (2) a 2,3-double bond combined with a 4-oxo group in the C-ring; and (3) hydroxyl groups at positions C-3 and C-5. The structure-activity relationships for flavonoids have been investigated in many chemical antioxidant activity assays (Silva, M. et al (2002) Free Radical Res. 36(11):1219-1227; Cao, G. et al (1997) Free Radical Biol. Med. 22(5):749-760; Rice-Evans, et al (1996) Free Radical Biol. Med. 20(7):933-935; Foti, M. et al (1996) J. Agric. Food Chem. 44:497-501; van Acker, S. A. et al (1996) Free Radical Biol. Med. 20(3):331-342; Arora, A. et al (1998) Free Radical Biol. Med. 24(9):1355-1363; Burda, S. and Oleszek, W. (2001). J. Agric. Food Chem. 49:2774-2779), and the required structural features for high activity are often those proposed by Bors et al. (Bors et al (1990), supra), but not always. Thus, structure-activity relationships depend on the protocol employed, and it is necessary to define them to predict activity under the investigative set of conditions.

Described herein in Example 3 are methods for measuring the antioxidant activity of several flavonoid compounds.

### B. Materials and Methods

### Chemicals

2′,7′-Dichlorofluorescin diacetate (DCFH-DA), fluorescein disodium salt, apigenin, (+)-catechin hydrate, chrysin, daidzein, (−)-epicatechin, (−)-epicatechin gallate (ECG), (−)-epigallocatechin (EGC), (−)-epigallocatechin gallate (EGCG), galangin, genistein, 6-hydroxy-2,5,7,8-tetramethylchoman-2-carboxylic acid (Trolox), kaempferol, luteolin, morin hydrate, naringenin, quercetin dihydrate, rutin hydrate, and taxifolin were purchased from Sigma-Aldrich, Inc. (St. Louis, Mo.). Myricetin and quercetin-3-β-D-glucoside (Q-3-G) were obtained from Fluka Chemical Corp. (Milwaukee, Wis.). Dimethyl sulfoxide was obtained from Fisher Scientific (Pittsburgh, Pa.), and 2,2′-azobis(2-amidinopropane) dihydrochloride (ABAP) was purchased

from Wako Chemicals USA, Inc. (Richmond, Va.). Methanol was obtained from Mallinckrodt Baker, Inc. (Phillipsburg, N.J.). The HepG2 cells were obtained from the American Type Culture Collection (ATCC) (Rockville, Md.). Williams' Medium E (WME) and Hanks' Balanced
Salt Solution (HBSS) were purchased from Gibco Life Technologies (Grand Island, N.Y.). Fetal bovine serum (FBS) was obtained from Atlanta Biologicals (Lawrenceville, Ga.).

### Preparation of Solutions

A 200 mmol/L stock solution of DCFHDA in methanol was prepared, aliquoted, and stored at −20° C. A 200 mmol/L ABAP stock solution in water was prepared, and aliquots were stored at −40° C. Working flavonoid solutions were prepared in dimethyl sulfoxide before further dilution in treatment medium (WME with 2 mM L-glutamine and 10 mmol/L Hepes). Final treatment solutions for cellular antioxidant activity assay contained 0.5% dimethyl sulfoxide, and solutions for cytotoxicity experiments contained 1% dimethyl sulfoxide.

### Cell Culture

HepG2 cells were cultured as described herein in Example 1.

### Cytotoxicity

The cytotoxicity of flavonoids toward HepG2 cells was measured, as described previously (Wolfe, K. L. and Liu, R. H. (2007), supra, Yoon, H. and Liu, R. H. (2007) J. Agric. Food Chem. 55:3167-3173). Briefly, HepG2 cells were seeded at 4×10⁴/well on a 96-well plate in 100 μL growth medium and incubated for 24 h at 37° C. The medium was removed, and the cells were washed with PBS. Flavonoids in 100 μL growth medium were applied to the cells, and the plates were incubated at 37° C. for 24 h. The medium was removed, and the cells were washed with PBS before a volume of 50 μL/well methylene blue staining solution (98% HBSS, 0.67% glutaraldehyde, 0.6% methylene blue) was applied to each well, and the plate was incubated at 37° C. for 1 h. The dye was removed, and the plate was immersed in fresh deionized water until the water was clear. The water was tapped out of the wells, and the plate was allowed to air-dry briefly before 100 μL of elution solution (49% PBS, 50% ethanol, 1% acetic acid) was added to each well. The microplate was placed on a benchtop shaker for 20 min to allow uniform

elution. The absorbance was read at 570 nm with blank subtraction using the MRX II Dynex spectrophotometer (Dynex Inc., Chantilly, Va.). The median cytotoxic concentration (CC₅₀) was calculated for each flavonoid.

### Cellular Antioxidant Activity (CAA) of Flavonoids

The CAA assay protocol is described herein in Example 2.

### Quantification of Cellular Antioxidant Activity (CAA)

Quantification was performed as described herein in Example 2.

### Measurement of Oxygen Radical-Scavenging Capacity (ORAC)

ORAC measurements were performed as described herein in Example 2.

### Octanol-Water Partition Coefficients (P)

Log P values for the selected flavonoids were estimated using the log P add-on for ChemSketch 10.0 (Advanced Chemistry Development, Inc., Toronto, ON, Canada).

### Statistical Analyses

All results are presented as mean±SD, and statistical analyses were performed using Minitab 15 (Minitab Inc., State College, Pa.). Differences between means were detected by ANOVA, followed by multiple comparisons using Fisher's least significant difference test. ANOVA was performed on log-transformed EC₅₀ values for CAA because the assumptions of normally distributed residuals and equal variances were not met by the untransformed data. Correlations were determined using linear regression. Results were considered to be significant when p<0.05.

### C. Results

### Efficacy of Selected Flavonoids in the CAA Assay

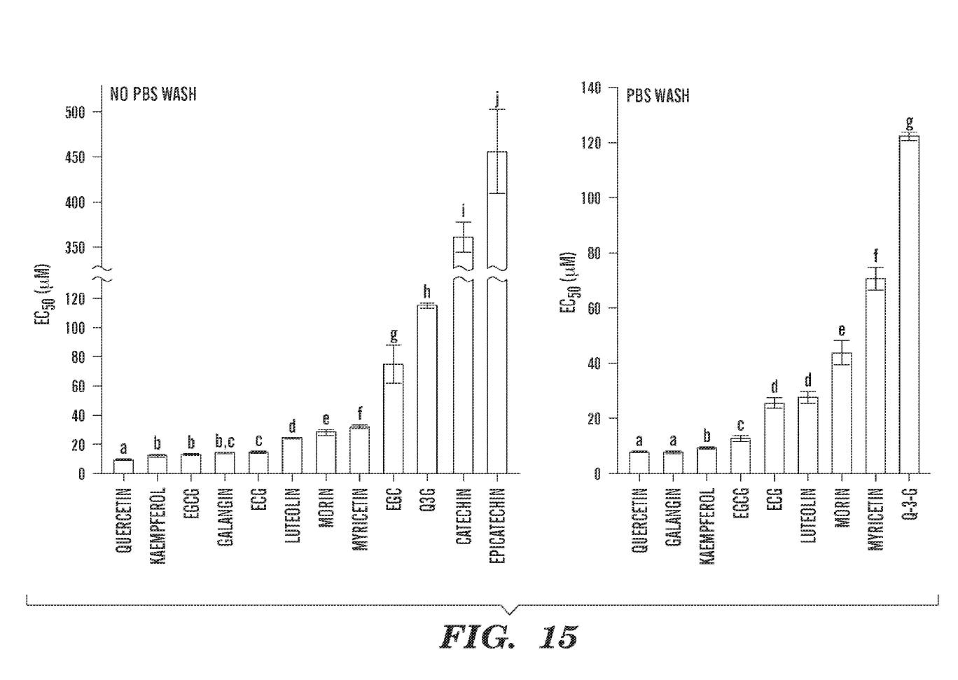

Nineteen flavonoids were evaluated for their cellular antioxidant activities using the methods described herein. The EC₅₀ values for those with quantifiable activity are depicted in FIG. 15, and the EC₅₀ values and their corresponding CAA values for each flavonoid tested are listed in Table 7. When the cells were not washed with PBS between flavonoid and ABAP treatments (no PBS wash protocol), quercetin had the highest activity, or lowest EC₅₀ value (p<0.05), followed by kaempferol, EGCG, and galangin, which had similar EC₅₀ values (p>0.05). The efficacies of the remaining flavonoids in the no PBS wash protocol were in the following order: ECG>luteolin>morin>myricetin>EGC>Q-3-G>catechin>epicatechin; taxifolin had low, but unquantifiable, activity.

When the HepG2 cells were washed with PBS between flavonoid and ABAP treatments (PBS wash protocol), the order of efficacy was quercetin=galangin>kaempferol>EGCG>ECG) luteolin>morin>myricetin>Q-3-G. EGC, catechin, epicatechin, and taxifolin had low activity at the concentrations tested. Genistein, daidzein, apigenin, naringenin, chrysin, and rutin had no activity in either protocol.

### Structure-Activity Relationships of Selected Flavonoids in CAA Assay

The molecular structures of flavonoids that dictate their efficacies in the cellular antioxidant activity were investigated. The generic structure of flavonoids is illustrated in FIG. 14. Flavonoids undergo extensive phase I and phase II metabolism within enterocytes upon absorption and other tissues after transport (Spencer, J. P. E. et al. (2004) Arch. Biochem. Biophys. 423(1):148-161), which will ultimately affect their bioactivities. The incorporation of cellular metabolism into the assay is one of the features that make the CAA an improvement over chemistry antioxidant activity assays. HepG2 cells were used because the results are similar to those obtained from intestine-like Caco-2 cells, but with much less variation (data not shown). Because flavonoid metabolism will influence efficacy, a flavonoid structure-activity examination was warranted as a first step toward characterizing the CAA assay and as a comparison of the CAA assay to chemistry antioxidant activity assays. The three structural features proposed to be essential for flavonoid antioxidant activity by Bors et al. (Bors, et al (1990), supra) sa B-ring o-dihydroxy group, a 2,3-double bond combined with a 4-oxo group in the C-ring, and a 3-hydroxyl groups were examined, as well as quercetin glycosylation and structures particular to isoflavones and flavanols.

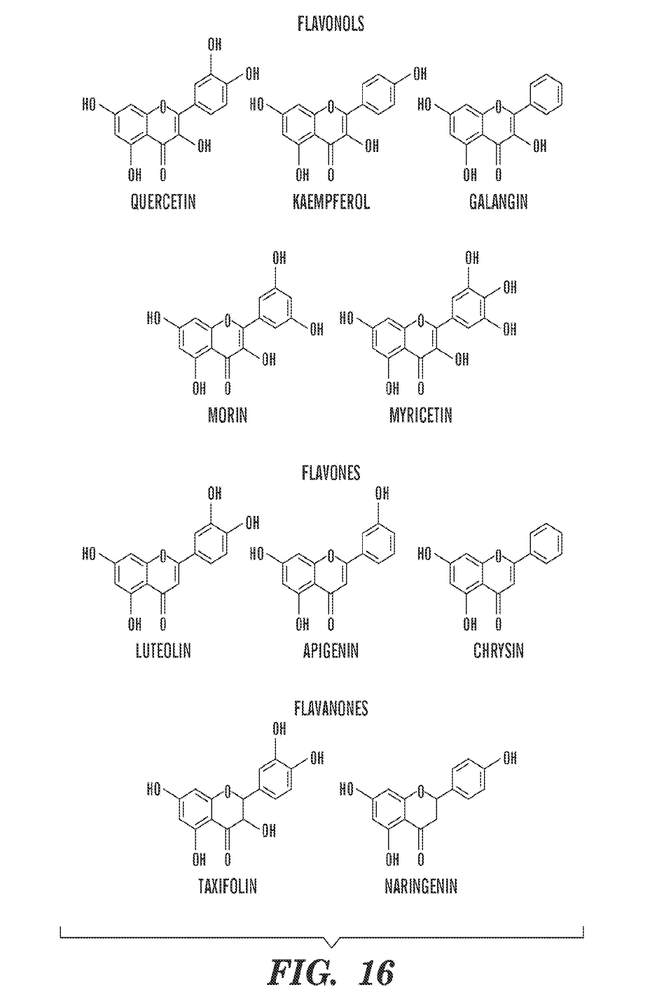

### B-Ring Hydroxylation of Flavonols, Flavones, and Flavanones (FIG. 16)

The number and positioning of the B-ring hydroxyl groups in flavonoids were important to cellular antioxidant activity. FIG. 16 shows the hydroxylation patterns of tested flavonoids from the flavonol, flavone, and flavanone subclasses. Of the flavonols tested in the no PBS wash protocol, quercetin, which has a 3′,4′-o-dihydroxyl group, had the highest activity (p<0.05) with an EC₅₀ of 8.93±0.44 μmol/L (Table 7). Kaempferol and galangin had similar efficacies, despite the lack of B-ring hydroxyl groups on galangin, and had only slightly higher EC₅₀ values, or slightly lower activities, than quercetin (p<0.05). Morin, which has two B-ring hydroxyl groups in the meta configuration, had much lower activity (EC₅₀=27.6±1.8 mol/L; p<0.05) than quercetin. The presence of a m-diphenolic moiety in the B-ring reduced activity compared to the ortho configuration in the TEAC assay, as well (Rice-Evans, C. A. et al (1996), supra). Myricetin has an extra 5′-hydroxyl group compared to quercetin and had even lower activity than morin (EC₅₀=31.1±1.0 μM). Of those tested, luteolin was the only flavone with activity in the CAA assay. The flavanone, taxifolin, had low but unquantifiable activity, whereas naringenin had none. In the PBS wash protocol, the flavonols with lowest EC₅₀ values for cellular antioxidant activity were quercetin (7.71±0.26 μmol/L) and galangin (7.56±0.46 μmol/L), followed closely by kaempferol. Morin, possessing 3′,5′-m-hydroxylation, had a higher EC₅₀ value (43.9±0.43 mol/L; p<0.05), and the addition of another hydroxyl group in myricetin further decreased activity. Again, only the flavone and flavanone with B-ring catechol groups had cellular antioxidant activity. These results indicate that a 3′,4′-o-dihydroxyl group is an indicator of substantial antioxidant activity for flavonoids in the CAA assay, especially for those not belonging to the flavonol subclass. This is consistent with previous reports that a B-ring catechol group is essential for high antioxidant activity in many different systems (Silva, M. M.; et al (2002), supra; Rice-Evans, C. A. et al (1996), supra; Foti, M et al (1996), supra; van Acker, S. A.; et al (1996), supra; Arora, A. et al (1998), supra; Burda, S. (2001), supra; Wang, et al (1999) Free Radical Biol. Med. 27(5-6):683-694). The higher antioxidant activity of flavonoids with an o-dihydroxyl group in the B-ring has been attributed to their greater radical stability through increased electron delocalization (Bors, W. et al (1990), supra) and intramolecular hydrogen bonding between the 3′- and 4′-hydroxyls (van Acker, S. A., et al (1996) Chem. Res. Toxicol. 9(8):1305-1312). An additional 5′-hydroxyl group in the B-ring, as seen in myricetin, has been shown to decrease antioxidant activity in other assays, which may be due to a pro-oxidant effect introduced by the pyrogallol group (van Acker, S. A.; et al (1996), supra).

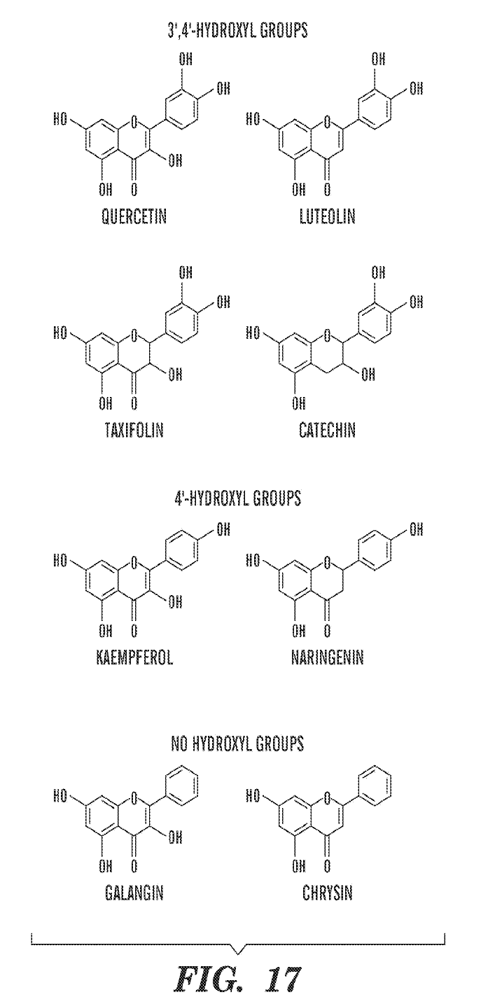

### Presence of 2,3-Double Bond, 4-Keto Group, and 3-Hydroxyl Moiety (FIG. 17)

The EC₅₀ values for flavonoids in the no PBS wash and PBS wash protocols showed similar trends (FIG. 15). For flavonoids with a B-ring catechol group (quercetin, luteolin, taxifolin, and catechin), the loss of any of the C-ring functional groups, the 2,3-double bond, 4-keto group, or 3-hydroxyl group, tended to result in a reduction of activity. When the 2,3-double bond conjugated to the 4-keto group was present, the absence of the 3-hydroxyl group, as in luteolin, moderately increased the EC₅₀ value and decreased cellular antioxidant activity. Removal of the 3-hydroxyl group from a flavonol introduces an approximately 20° twist of the B-ring relative to the A- and C-rings, and nonplanar molecules cannot delocalize electrons across the molecule effectively and have less scavenging activity (van Acker, S. A. et al (1996) Chem. Res. Toxicol. 9(8):1305-1312). In addition, hydrogen bonding between the 4-keto and 3-hydroxyl or 5-hydroxyl groups stabilizes flavonoid radicals van Acker, S. A. et al (1996) Chem. Res. Toxicol. 9(8):1305-1312). The 4-keto group, along with the 5-hydroxyl moiety, is the most important site for the chelation of transition metal ions, which can catalyze oxidative chain reactions (Mira, L. et al (2002) Free Radical Res. 36(11):1199-1208). The 2,3-double bond combined with the 4-keto group delocalizes electrons from the B-ring (Bors, W. et al (1990), supra), and the loss of one or both characteristics dramatically reduced cellular antioxidant activity. This is demonstrated when the EC₅₀ values of quercetin to taxifolin and catechin and that of kaempferol to naringenin are compared. Similar trends were seen in the TEAC assay (Rice-Evans, C. A. et al (1996), supra); however, C-ring unsaturation did not affect antioxidant activity in the ORAC assay (Silva, M. M. et al, (2002), supra). The 2,3-double bond may be more important for cellular antioxidant activity than the 3-hydroxyl group because a greater increase in EC₅₀ values accompanied the loss of that structural feature; compared to quercetin, luteolin maintained higher activity than taxifolin. In the absence of B-ring hydroxylation, the 3-hydroxyl group was important to antioxidant activity, as shown by the low EC₅₀ value for galangin and the absence of activity by chrysin.

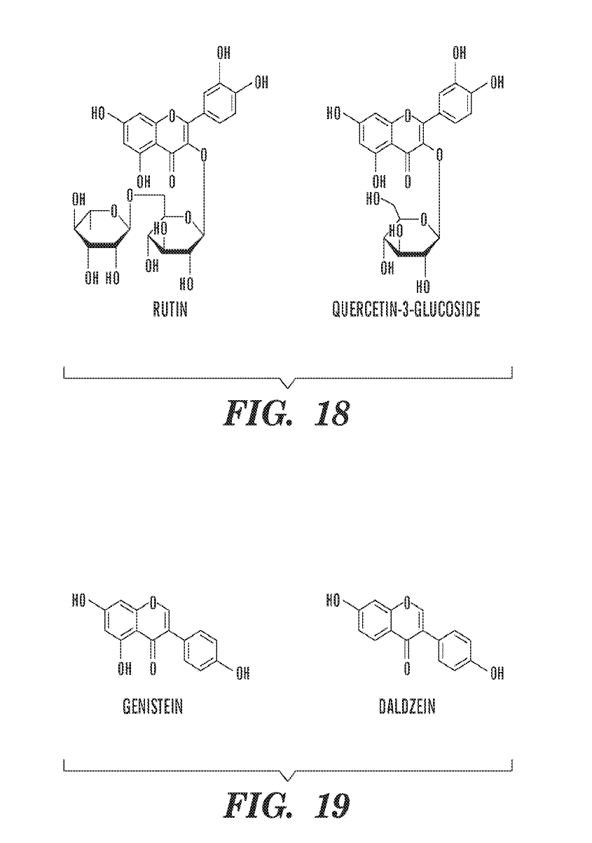

### Glycosylation (FIG. 18)

The 3-glycosylation of quercetin dramatically affected its antioxidant activity in the CAA assay, and also the type of esterified sugar was important. Q-3-G maintained a low amount of activity, whereas rutin had none. It is interesting to note that rutin had the highest antioxidant activity in the ORAC assay (Table 8). The much higher EC₅₀ values of Q-3-G and rutin compared to luteolin, which has the same base structure, could possibly be explained by the greater twist of the B-ring compared to the A- and C-rings introduced by glycosylation, as the torsion angle for rutin is almost 30°, whereas the torsion angle for luteolin is only around 20° (van Acker, S. A. et al (1996) Chem. Res. Toxicol. 9 (8):1305-1312).

### Isoflavones (FIG. 19)

The isoflavones genistein and daidzein had no activity in the CAA assay (Table 7). Genistein and daidzein were effective reducers of the ATBS•+cation in the TEAC assay, but performed poorly at reducing the Fe(III) complex in the FRAP assay, quenching galvinoxyl radicals, and inhibiting microsomal lipid peroxidation (Mitchell, J. H. et al (1998) Arch. Biochem. Biophys. 360(1):142-148). The experiments showed that isoflavones are poor hydrogen donors and have activities only at levels beyond which are achievable in vivo. Guo et al. (Guo, Q. et al (2002) Toxicology 179(1-2):171-180) also reported limited antioxidant activity of isoflavones against a variety of oxidants and free radicals, and genistein had low efficacy against ABAP-induced oxidation of liposomes (Silva, M. M. et al (2002), supra). In agreement, another earlier study found genistein to be a poor antioxidant in liposome and micelle systems oxidized using ABAP (Record, I. R. et al (1995) J. Nutr. Biochem. 6(9):481-485), although it was an effective antioxidant in assays where hydrogen peroxide or iron was involved in the oxidation, despite not being a good metal chelator. The isoflavone metabolite, equol, which is identical to daidzein except for having a saturated C-ring, had much higher antioxidant activity against Fe(II)-, Fe(III)-, and ABAP induced oxidation of liposomes compared to genistein and daidzein (Arora, A.; et al (1998) Arch. Biochem. Biophys. 356(2):133-141). The absence of a 2,3-double bond could be a major determinant of isoflavone antioxidant activity. It is not surprising, therefore, that genistein and daidzein did not have activity in the CAA assay, which involves ABAP-induced oxidation of a cell membrane. Further research is needed to determine if isoflavones with no 2,3-double bond have cellular antioxidant activity.

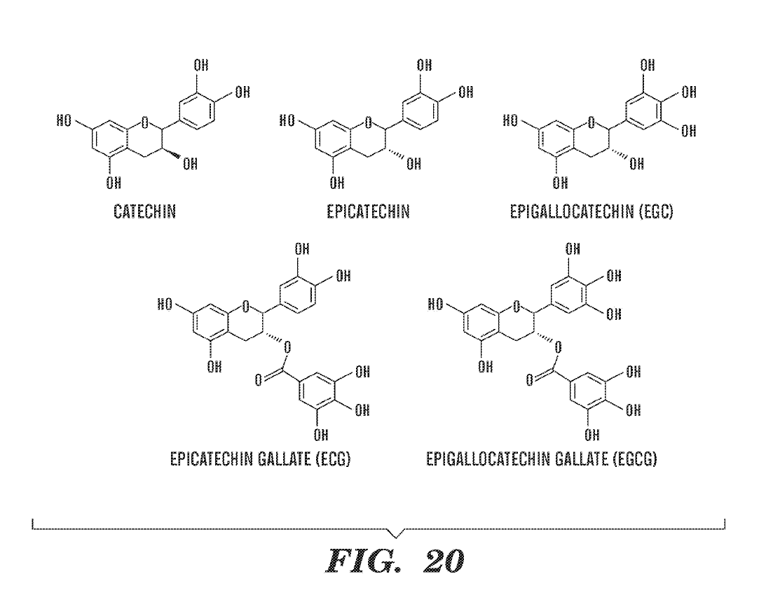

### Flavanols (Catechins) (FIG. 20)

Catechin and epicatechin had low activity (EC₅₀=360±17 and 457±47 μM, respectively) in the no PBS wash protocol and very low, unquantifiable, activity in the PBS wash protocol. In both protocols, the presence of a galloyl group in the flavanols EGCG and ECG imparted them with very high activity, and low EC₅₀ values, in the CAA assay compared to catechin, epicatechin, and EGC. An additional B-ring hydroxyl group at the 5′-position gave EGC a much lower EC₅₀ value than catechin and epicatechin in the no PBS wash protocol and slightly increased the activity of EGCG over ECG in both methods (FIG. 15). This trend is similar to that for the scavenging activities of flavanols against ABAP-generated radicals in phosphate buffer (Guo, Q., et al (1999) Biochim. Biophys. Acta 1427(1):13-23) and in the TEAC assay (Rice-Evans, C. A., et al (1996), supra). Correlations between CAA and Lipophilicity. Log P (octanol-water partitioning coefficient) values were estimated using computer software (Table 8) and compared to the EC₅₀ values for CAA. Some of the most lipophilic compounds, apigenin, genistein, chrysin, and daidzein, had no cellular antioxidant activity, and so were not included in the correlation analysis. Many of the more hydrophilic flavonoids, such as epicatechin, catechin, Q-3-G, rutin, and taxifolin, also had low or no cellular antioxidant activity. The EC₅₀ values obtained from the PBS wash CAA method were not related to the log P values of the flavonoids (R²=0.232, p>0.05, n=9), but the EC₅₀ values for the cellular antioxidant activity of flavonoids in the no PBS wash protocol were significantly negatively correlated to their lipophilicity (R²=0.864, p<0.001, n=11). The antioxidant activity values from the no PBS wash protocol may have been more reflective of the interactions of the flavonoids with the cell membrane, as the PBS wash likely removed flavonoids with weak interactions, leaving only those that were taken up by the cells, deeply embedded in the lipid bilayer, or tightly bound to the cell membranes to scavenge peroxyl radicals. Octanol-water partitioning coefficients are a measure of lipophilicity and are commonly used to predict the distribution and fate of toxins and pharmaceuticals in the body and chemicals in the environment (Crosby, D. G. (1998) Environmental Toxicology and Chemistry; Oxford University Press: New York). Glycosylation and hydroxylation both decrease the lipophilicity of flavonoids, and sugar esterification is the greater modulator (Rothwell, J. A.; et al (2005) J. Agric. Food Chem. 53(11):4355-4360). The lipophilicity of flavonoids may play a role in their accessibility to free radicals, so membrane partitioning is thought to be important in dictating their antioxidant activity (Brown, J. E., et al (1998) Biochem. J. 330(Part 3):1173-1178; Saija, A., et al (1995) Free Radical Biol. Med. 19(4):481-486). Flavonoids with very high or very low lipophilicity had low antioxidant activities against Fe(III)-induced lipid peroxidation of mouse liver microsomes (Yang, B. et al (2001) Anal. Sci. 17(5):599-604), and that may also be the case in the CAA assay. As was observed in the ABAP-induced oxidation of linolenic acid in micelles (Foti, M., et al (1996), supra), flavonoids with higher log P values tended to have greater antioxidant activity, but structural features dictated the activities of compounds with similar lipophilicities.

### Correlations Between CAA and ORAC Values

The ORAC assay measures the ability of antioxidants to scavenge peroxyl radicals generated by ABAP and delay the decay in fluorescence of the fluorescein probe. The ORAC values for selected flavonoids are listed in Table 8. Rutin, genistein, and catechin had the highest activities in the ORAC assay (13.7±1.7, 13.4±2.8, and 12.4±4.0 μmol of TE/μmol, respectively; p<0.05), followed by apigenin, taxifolin, and naringenin, which were not significantly different from catechin (p>0.05). Galangin, EGC, chrysin, myricetin, EGCG, and morin had the lowest antioxidant activities in the ORAC assay (2.63±1.31, 3.11±0.73, 3.79±0.67, 4.55±0.50, 4.55±0.40, and 6.12±1.95 μmol of TE/μmol, respectively; p<0.05). The antioxidant activity ranking of tested flavonoids in the ORAC assay was different from the results reported by Cao et al. (Cao, G.; et al (1997), supra) and Ou et al. (Ou, B. et al (2001) J. Agric. Food Chem. 49: 4619-4626), but was more in agreement with the ranking reported by Aaby et al. (Aaby, K.; et al (2004) J. Agric. Food Chem. 52:4595-4603). Our ORAC values of tested flavonoids also tended to be higher than the values presented in those studies. Variations in results can likely be explained by differences in protocols. Cao et al. (Cao, G.; et al (1997), supra) and Aaby et al. (Aaby, K.; et al (2004), supra) both used β-phycoerythrin, not fluorescein, as a probe. The Prior group later showed that the values obtained from using a fluorescein probe tended to be higher compared to those from using β-phycoerythrin and that the compounds with the highest activities using one probe did not always have the highest activities with the other (Ou, B. et al (2001), supra). In addition, the reagent concentrations, solvents used to dissolve the flavonoids, pH of the buffers, and reaction times differed. The degree of hydroxylation has been cited as the biggest determinant of antioxidant activity in the ORAC assay (Silva, M. M.; et al (2002), supra; Cao, G.; et al (1997), supra); however, our ORAC data do not support that idea. To attempt to explain the CAA of flavonoids by their radical scavenging abilities in a simple system, their CAA and ORAC values were compared. There was no significant association between flavonoid ORAC and CAA values (R²=0.214, p>0.05, n=12 for the no PBS wash protocol; R²=0.080, p>0.05, n=9 for the PBS wash protocol). This was also found when the relationship between ORAC and the prevention of oxidative stress in HepG2 cells for broccoli extracts was examined (Eberhardt, M. V. et al (2005) J. Agric. Food Chem. 53:7421-7431). In fact, many of the flavonoids with the highest ORAC values had no CAA (rutin, genistein, apigenin, and naringenin) or low CAA (catechin, taxifolin, and epicatechin). Conversely, galangin and EGCG were among the flavonoids with the lowest ORAC values, but they exhibited high activity in the CAA assay. Quercetin, kaempferol, and luteolin, which had high cellular antioxidant activity, were only moderately effective in the ORAC assay compared to the other tested flavonoids. The lack of correlation between the CAA and ORAC assays is likely due to the biological components of the CAA assay; because it monitors oxidative stress in cells, not a test tube, it accounts for some aspects of cell uptake, distribution, and metabolism of antioxidant compounds.

### Abbreviations Used

ABAP, 2,2′-azobis(2-amidinopropane) dihydrochloride; CAA, cellular antioxidant activity; CV, coefficient of variation; DCF, dichlorofluorescein; DCFH, dichlorofluorescin; DCFH-DA, dichlorofluorescin diacetate; DPPH, 2,2-diphenyl-picrylhydrazyl; EC₅₀, median effective concentration; EGCG, epigallocatechin gallate; FRAP, Ferric Reducing/Antioxidant Parameter; GAE, gallic acid equivalents; HBSS, Hanks' Balanced Salt Solution; ORAC, Oxygen-Radical Absorbance Capacity; PBS, phosphate-buffered saline; PSC, Peroxyl Radical Scavenging Capacity; QE, quercetin equivalents; ROS, reactive oxygen species; TEAC, Trolox Equivalent Antioxidant Capacity; TOSC, Total Oxyradical Scavenging Capacity; TRAP, Total Radical-Trapping Antioxidant Parameter; WME, Williams' Medium E.

TABLE 1
EC₅₀ values for the inhibition of peroxyl radical-induced DCFH oxidation by
selected pure phytochemical compounds and fruits (mean ± SD, n = 3) and their cytotoxic
concentrations.

| | No PBS Wash | | PBS Wash | | ²Cytotoxicity |
|---|---|---|---|---|---|
| Compound | EC₅₀ (μM) | CV (%) | EC₅₀ (μM) | CV (%) | (μM) |
| ¹Quercetin | 5.92 ± 0.07 | 1.18 | 5.09 ± 0.19 | 3.65 | >20 |
| ¹Kaempferol | 7.85 ± 0.51 | 6.53 | 6.31 ± 0.21 | 3.34 | 30 |
| EGCG | 14.0 ± 1.0 | 7.39 | 15.8 ± 0.4 | 2.81 | >100 |
| Myricetin | 16.1 ± 1.70 | 10.6 | 15.4 ± 0.5 | 2.96 | 200 |
| ¹Luteolin | 26.1 ± 0.26 | 1.01 | 23.1 ± 1.0 | 4.48 | 20 |
| ¹Gallic acid | 65.4 ± 7.3 | 11.1 | 335 ± 26 | 7.81 | >500 |
| Ascorbic acid | 67.5 ± 9.4 | 14.0 | >500 | | >500 |
| ¹Caffeic acid | 95.3 ± 15.3 | 16.1 | 525 ± 38 | 7.25 | >500 |
| Catechin | 292 ± 32 | 11.0 | >500 | | >500 |
| Epicatechin | >200 | | >600 | | >500 |
| Ferulic acid | >250 | | >500 | | >500 |
| Phloretin | >25 | | >25 | | 25 |
| Resveratrol | >40 | | >40 | | 40 |
| Taxifolin | >150 | | >150 | | 150 |
| Fruit | EC₅₀ (mg/mL) | CV (%) | EC₅₀ (mg/mL) | CV (%) | (mg/mL) |
| ¹Blueberry | 3.440 ± 0.239 | 6.94 | 10.81 ± 0.44 | 4.09 | 60 |
| ¹Cranberry | 11.31 ± 0.29 | 2.59 | 36.17 ± 1.20 | 3.31 | 60 |
| ¹Apple | 21.31 ± 3.34 | 16.0 | 38.60 ± 3.26 | 8.45 | >100 |
| ¹Red grape | 24.49 ± 1.73 | 7.05 | 42.33 ± 2.22 | 5.23 | >100 |
| ¹Green grape | 62.89 ± 3.19 | 5.07 | 53.01 ± 3.12 | 5.89 | >100 |

¹EC₅₀ values for no PBS wash and PBS wash are significantly different (p < 0.05)
²Dose at which the cell number is reduced by more than 10% after 24 h treatment

TABLE 1
EC₅₀ values for the inhibition of peroxyl radical-induced DCFH
oxidation by selected pure phytochemical compounds and fruits
(mean ± SD, n = 3) and their cytotoxic concentrations.

| | No PBS Wash | | PBS Wash | | ²Cyto- |
|---|---|---|---|---|---|
| | | CV | | CV | toxicity |
| Compound | EC₅₀ (μM) | (%) | EC₅₀ (μM) | (%) | (μM) |
| ¹Quercetin | 5.92 ± 0.07 | 1.18 | 5.09 ± 0.19 | 3.65 | >20 |
| ¹Kaempferol | 7.85 ± 0.51 | 6.53 | 6.31 ± 0.21 | 3.34 | 30 |
| EGCG | 14.0 ± 1.0 | 7.39 | 15.8 ± 0.4 | 2.81 | >100 |
| Myricetin | 16.1 ± 1.70 | 10.6 | 15.4 ± 0.5 | 2.96 | 200 |
| ¹Luteolin | 26.1 ± 0.26 | 1.01 | 23.1 ± 1.0 | 4.48 | 20 |
| ¹Gallic acid | 65.4 ± 7.3 | 11.1 | 335 ± 26 | 7.81 | >500 |
| Ascorbic | 67.5 ± 9.4 | 14.0 | >500 | | >500 |
| acid | | | | | |
| ¹Caffeic | 95.3 ± 15.3 | 16.1 | 525 ± 38 | 7.25 | >500 |
| acid | | | | | |
| Catechin | 292 ± 32 | 11.0 | >500 | | >500 |
| Epicatechin | >200 | | >600 | | >500 |
| Ferulic acid | >250 | | >500 | | >500 |
| Phloretin | >25 | | >25 | | 25 |
| Resveratrol | >40 | | >40 | | 40 |
| Taxifolin | >150 | | >150 | | 150 |
| | | CV | | CV | |
| Fruit | EC₅₀ (mg/mL) | (%) | EC₅₀ (mg/mL) | (%) | (mg/mL) |
| ¹Blueberry | 3.440 ± 0.239 | 6.94 | 10.81 ± 0.44 | 4.09 | 60 |
| ¹Cranberry | 11.31 ± 0.29 | 2.59 | 36.17 ± 1.20 | 3.31 | 60 |
| ¹Apple | 21.31 ± 3.34 | 16.0 | 38.60 ± 3.26 | 8.45 | >100 |
| ¹Red grape | 24.49 ± 1.73 | 7.05 | 42.33 ± 2.22 | 5.23 | >100 |
| ¹Green | 62.89 ± 3.19 | 5.07 | 53.01 ± 3.12 | 5.89 | >100 |
| grape | | | | | |

¹EC₅₀ values for no PBS wash and PBS wash are significantly different (p < 0.05)
²Dose at which the cell number is reduced by more than 10% after 24 h treatment

TABLE 2
Variation among EC₅₀ values for representative pure phytochemical
compounds and fruit extracts obtained when no PBS wash was
performed between antioxidant and ABAP treatments (mean ± SD).

| | | Intraexperimental | | Interexperimental | |
|---|---|---|---|---|---|
| Compound | Trial | ¹EC₅₀ (μM) | CV (%) | EC₅₀ (μM) | CV (%) |
| Quercetin | 1 | 5.92 ± 0.07 | 1.18 | | |
| | 2 | 6.07 ± 0.25 | 4.20 | | |
| | 3 | 6.21 ± 0.24 | 3.86 | | |
| | 4 | 5.28 ± 0.23 | 4.40 | | |
| | 5 | 5.98 ± 0.18 | 3.01 | 5.89 ± 0.36 | 6.06 |
| Gallic Acid | 1 | 65.4 ± 7.3 | 11.13 | | |
| | 2 | 51.3 ± 3.0 | 5.77 | | |
| | 3 | 77.3 ± 4.9 | 6.36 | | |
| | 4 | 63.3 ± 2.7 | 4.19 | 64.3 ± 10.7 | 16.6 |
| Fruit | Trial | ¹EC₅₀ (mg/mL) | CV (%) | EC₅₀ (mg/mL) | CV (%) |
| Blueberry | 1 | 3.44 ± 0.24 | 6.94 | | |
| | 2 | 3.49 ± 0.39 | 11.17 | | |
| | 3 | 3.83 ± 0.29 | 7.56 | | |
| | 4 | 2.20 ± 0.16 | 7.39 | | |
| | 5 | 2.60 ± 0.06 | 2.13 | 3.11 ± 0.68 | 22.0 |

¹n = 3

TABLE 3
Variation among EC₅₀ values for representative pure phytochemical
compounds and fruit extracts obtained when a PBS wash was
performed between antioxidant and ABAP treatments (mean ± SD).

| | | Intraexperimental | | Interexperimental | |
|---|---|---|---|---|---|
| Compound | Trial | ¹EC₅₀ (μM) | CV (%) | EC₅₀ (μM) | CV (%) |
| Quercetin | 1 | 5.55 ± 0.09 | 1.60 | | |
| | 2 | 4.48 ± 0.18 | 3.94 | | |
| | 3 | 5.40 ± 0.20 | 3.73 | | |
| | 4 | 5.09 ± 0.19 | 3.65 | | |
| | 5 | 5.06 ± 0.19 | 3.77 | 5.12 ± 0.58 | 11.3 |
| Gallic Acid | 1 | 289 ± 12 | 4.05 | | |
| | 2 | 270 ± 32 | 11.78 | | |
| | 3 | 350 ± 21 | 6.14 | | |
| | 4 | 347 ± 37 | 10.60 | | |
| | 5 | 335 ± 26 | 7.81 | 318 ± 36 | 11.4 |
| Fruit | Trial | ¹EC₅₀ (mg/mL) | CV (%) | EC₅₀ (mg/mL) | CV (%) |
| Blueberry | 1 | 11.2 ± 1.2 | 11.08 | | |
| | 2 | 10.3 ± 0.8 | 7.95 | | |
| | 3 | 11.6 ± 0.4 | 3.45 | | |
| | 4 | 9.52 ± 0.48 | 5.05 | | |
| | 5 | 10.8 ± 0.4 | 4.09 | 10.7 ± 0.8 | 7.56 |

¹n = 3

TABLE 4
Comparison of antioxidant quality of fruit extracts using the
cellular antioxidant activity (CAA) assay (mean ± SD, n = 3)

| | No PBS Wash | PBS Wash |
|---|---|---|
| | ¹Cellular antioxidant | ¹Cellular antioxidant |
| | activity (μmol QE/100 | activity (μmol QE/100 |
| Fruit | μmol total phenolics) | μmol total phenolics) |
| Blueberry | ^{a}8.70 ± 0.19 | ^{A}1.82 ± 0.07 |
| Cranberry | ^{b}3.36 ± 0.09 | ^{C, D}0.914 ± 0.03 |
| Apple | ^{b}3.07 ± 0.45 | ^{B}1.45 ± 0.12 |
| Red grape | ^{c}1.67 ± 0.12 | ^{D}0.839 ± 0.044 |
| Green grape | ^{d}1.04 ± 0.05 | ^{C}0.973 ± 0.057 |

¹Values with no letters in common are significantly different (p < 0.05)

TABLE 5
Cellular Antioxidant Activities of Selected Fruits Expressed
as EC₅₀ and CAA Values (Mean ± SD, n = 3)

| | no PBS wash | | PBS wash | | cytotoxicity |
|---|---|---|---|---|---|
| fruit | EC₅₀^{b} (mg/mL) | CAA (μmol of QE/100 g) | EC₅₀^{b} (mg/mL) | CAA (μmol of QE/100 g) | CC₅₀^{c} (mg/mL) |
| wild blueberry | 2.53 ± 0.10 | 292 ± 11 | 6.77 ± 1.06 | 74.1 ± 12.5 | >150 |
| pomegranate^{a} | 2.95 ± 0.11 | 250 ± 10 | 3.03 ± 0.07 | 163 ± 3.6 | >150 |
| blackberry^{a} | 3.19 ± 0.15 | 232 ± 11 | 3.21 ± 0.14 | 154 ± 6.8 | >150 |
| strawberry | 5.46 ± 0.66 | 136 ± 18 | 11.8 ± 0.9 | 42.2 ± 3.3 | >150 |
| blueberry | 5.95 ± 1.33 | 128 ± 30 | 27.0 ± 6.2 | 19.0 ± 4.7 | >150 |
| raspberry | 6.52 ± 0.60 | 114 ± 11 | 14.2 ± 0.9 | 35.0 ± 2.3 | >150 |
| cranberry^{a} | 15.6 ± 2.3 | 47.9 ± 6.5 | 14.7 ± 0.8 | 33.6 ± 20 | >150 |
| plum | 22.9 ± 5.5 | 33.5 ± 8.6 | 38.3 ± 0.3 | 12.9 ± 0.1 | >150 |
| cherry | 27.3 ± 3.9 | 27.4 ± 4.1 | 73.0 ± 7.7 | 6.81 ± 0.8 | >150 |
| apple^{a} | 34.4 ± 6.0 | 21.9 ± 4.0 | 29.0 ± 3.4 | 17.2 ± 2.0 | >150 |
| red grape^{a} | 45.3 ± 1.4 | 16.3 ± 0.5 | 39.6 ± 5.5 | 12.6 ± 1.8 | >150 |
| kiwifruit | 46.4 ± 7.2 | 18.1 ± 2.6 | 108 ± 8 | 4.58 ± 0.31 | 76.1 ± 4.6 |
| mango | 48.5 ± 4.6 | 15.3 ± 1.5 | 78.0 ± 2.6 | 6.33 ± 0.21 | >150 |
| pineapple | 49.8 ± 2.6 | 14.8 ± 0.8 | NQ | | >150 |
| orange | 54.0 ± 2.8 | 13.7 ± 0.7 | NQ | | 68.5 ± 14.9 |
| lemon | 60.3 ± 4.9 | 12.3 ± 1.0 | 134 ± 6 | 3.68 ± 0.16 | ND |
| grapefruit | 63.4 ± 3.2 | 11.6 ± 0.6 | NQ | | 63.9 ± 4.3 |
| peach^{a} | 78.2 ± 6.4 | 9.47 ± 0.82 | 81.7 ± 21.7 | 6.31 ± 1.53 | >150 |
| pear^{a} | 101 ± 10 | 7.35 ± 0.67 | 96.5 ± 7.2 | 5.13 ± 0.40 | >150 |
| nectarine | 108 ± 13 | 6.91 ± 0.89 | NQ | | >150 |
| honeydew | 183 ± 12 | 4.03 ± 0.28 | NQ | | >150 |
| avocado | 207 ± 17 | 3.58 ± 0.29 | NQ | | 24.3 ± 0.1 |
| cantaloupe | 209 ± 21 | 3.54 ± 0.35 | NQ | | >150 |
| banana | 235 ± 16 | 3.15 ± 0.21 | NQ | | >150 |
| watermelon | NQ | | NQ | | >150 |

^{a}EC₅₀ values obtained from the no PBS wash and PBS wash protocols are not significantly different (p > 0.05).
^{b}NQ, EC₅₀ values are not quantifiable due to low activity.
^{c}ND, CC₅₀ values are not quantifiable due to lock of dose-response.

TABLE 6
Cellular Antioxidant Quality of Fruit Phenolics in the Cellular
Antioxidant Activity Assay (Mean ± SD, n = 3)

| | cellular antioxidant quality^{d} | |
|---|---|---|
| | (μmol of QE/100 μmol of phenolics) | |
| fruit | no PBS wash | PBS wash |
| pomegranate^{a} | 12.6 ± 0.5 a | 8.2 ± 0.2 a |
| wild blueberry^{b} | 11.6 ± 0.4 b | 2.9 ± 0.5 c |
| strawberry | 9.9 ± 1.3 c | 3.0 ± 0.2 c |
| blackberry^{a} | 9.5 ± 0.4 d | 6.3 ± 0.3 b |
| raspberry^{b} | 8.1 ± 0.8 d | 2.5 ± 0.2 d |
| blueberry^{b} | 7.7 ± 1.8 d | 1.1 ± 0.3 gh |
| kiwifruit | 4.5 ± 0.7 e | 1.3 ± 0.1 g |
| honeydew^{c} | 4.4 ± 0.3 e | |
| mango | 4.2 ± 0.4 e | 1.7 ± 0.1 ef |
| lemon | 4.1 ± 0.3 e | 1.2 ± 0.1 gh |
| orange^{c} | 4.1 ± 0.2 e | |
| cantaloupe^{c} | 3.8 ± 0.4 e | |
| pineapple^{c} | 3.2 ± 0.2 f | |
| cherry | 3.1 ± 0.5 fg | 0.8 ± 0.1 i |
| cranberry^{a} | 2.8 ± 0.4 fg | 2.0 ± 0.1 e |
| grapefruit^{c} | 2.8 ± 0.1 fg | |
| avocado^{c} | 2.5 ± 0.2 fgh | |
| apples^{a} | 2.4 ± 0.2 fgh | 1.9 ± 0.2 e |
| Plum | 2.4 ± 0.6 ghi | 0.9 ± 0.0 hi |
| peach^{a} | 2.2 ± 0.2 ghi | 1.5 ± 0.4 fg |
| nectarine^{c} | 1.8 ± 0.2 hij | |
| red grape^{a} | 1.7 ± 0.1 hij | 1.3 ± 0.2 g |
| pear^{a} | 1.3 ± 0.1 ij | 0.9 ± 0.1 hi |
| banana^{c} | 1.0 ± 0.1 j | |
| watermelon | | |

^{a}Normalized cellular antioxidant quality from no PBS wash protocol is significantly lower than normalized cellular antioxidant quality from PBS wash protocol (p < 0.05).
^{b}Normalized cellular antioxidant quality from no PBS wash protocol is significantly higher than normalized antioxidant quality from PBS wash protocol (p < 0.05).
^{c}Cellular antioxidant quality for no PBS wash protocol is significantly different from zero (p < 0.05).
^{d}Values in each column with no letters in common are significantly different (p < 0.05).

TABLE 7
EC₅₀ and CAA values for flavonoids in the CAA assay (Mean ± SD, n = 3)

| | no PBS wash | | PBS wash | | |
|---|---|---|---|---|---|
| | EC₅₀ | CAA | EC₅₀ | CAA | cytotoxicity |
| flavonoid | (μmol/L) | (μmol of QE/100 μmol) | (μmol/L) | (μmol of QE/100 μmol) | CC₅₀ (μM) |
| quercetin | 8.93 ± 0.44 | 99.1 ± 4.8 | 7.71 ± 0.26 | 105.7 ± 3.7 | >100 |
| kaempferol | 11.9 ± 0.8 | 74.6 ± 4.8 | 9.57 ± 0.27 | 85.1 ± 2.4 | >100 |
| EGCG | 12.3 ± .7 | 72.1 ± 3.8 | 12.8 ± 0.9 | 63.8 ± 4.4 | >100 |
| galangin | 13.3 ± .2 | 66.3 ± 1.1 | 7.56 ± 0.46 | 107.8 ± 6.4 | >80 |
| ECG | 14.2 ± 0.4 | 62.1 ± 1.9 | 25.6 ± 1.8 | 31.9 ± 2.2 | >200 |
| luteolin | 23.8 ± .0 | 37.1 ± 0.0 | 27.7 ± 2.0 | 29.5 ± 2.1 | >80 |
| morin | 27.6 ± 1.8 | 32.1 ± 2.1 | 43.9 ± 4.3 | 18.6 ± 18 | >200 |
| myricetin | 31.1 ± 1.0 | 28.4 ± 0.9 | 70.7 ± 4.2 | 11.5 ± 0.7 | >200 |
| EGC | 74.2 ± 12.9 | 12.1 ± 2.2 | >100 | | >200 |
| Q-3-G | 115 ± 2 | 7.7 ± 0.1 | 122 ± 1 | 6.6 ± 0.1 | >200 |
| catechin | 360 ± 17 | 2.5 ± 0.1 | >800 | | >1000 |
| epicatechin | 457 ± 47 | 1.9 ± 0.2 | >800 | | >1000 |
| taxifolin | >200 | | >200 | | >200 |
| genistein | no activity | | no activity | | >100 |
| daidzein | no activity | | no activity | | >100 |
| apigenin | no activity | | no activity | | >80 |
| naringenin | no activity | | no activity | | >200 |
| chrysin | no activity | | no activity | | >200 |
| rutin | no activity | | no activity | | >200 |

TABLE 8
ORAC and Log P values for selected flavonoids

| flavonoid | ORAC^{a, b} (μmol of TE/μmol) | lipophilicity (log P) |
|---|---|---|
| quercetin | 8.04 ± 2.37 cde | 2.07 |
| kaempferol | 7.19 ± 1.29 def | 2.05 |
| EGCG | 4.55 ± 0.40 fgh | 2.08 |
| galangin | 2.63 ± 1.31 gh | 2.83 |
| ECG | 7.71 ± 1.57 cde | 2.67 |
| luteolin | 8.55 ± 0.85 cde | 2.4 |
| morin | 6.12 ± 1.95 efg | 2.62 |
| myricetin | 4.55 ± 0.50 fgh | 2.11 |
| EGC | 3.11 ± 0.73 cde | ND^{c} |
| Q-3-G | 8.11 ± 1.86 cde | 1.75 |
| catechin | 12.4 ± 4.0 ab | 0.49 |
| epicatechin | 9.14 ± 1.31 cd | 0.49 |
| taxifolin | 9.74 ± 1.20 bcd | 1.82 |
| genistein | 13.4 ± 2.8 a | 2.96 |
| daidzein | 8.52 ± 2.11 cde | 2.78 |
| apigenin | 10.7 ± 1.5 bc | 3.04 |
| naringenin | 9.23 ± 2.79 bcd | 2.42 |
| chrysin | 3.79 ± 0.67 gh | 2.88 |
| rutin | 13.7 ± 1.7 a | 1.76 |

^{a}Mean ± SD, n ≧ 3.
^{b}Values with no letter in common are significantly different (p < 0.05).
^{c}No data available.

1. A method of measuring antioxidant capacity of a test compound, the method comprising the steps of:
(a) contacting a cultured cell with 2′,7′-dichlorofluorescin diacetate in the presence and absence of a test compound, wherein said 2′,7′-dichlorofluorescin diacetate enters said cell and is cleaved to 2′,7′-dichlorofluorescin;
(b) contacting said cell with a peroxyl radical initiator;
(c) measuring fluorescence in an emission wavelength of 2′,7′-dichlorofluorescein at a plurality of time points; and
(d) determining the area-under-the-curve of a graph plotting 2′,7′-dichlorofluorescein diacetate fluorescence vs. time for said cultured cell in the presence and absence of said test compound,
wherein a decrease in said area-under-the-curve in the presence of said test compound, relative to said area-under-the-curve in the absence of said test compound indicates antioxidant capacity of said test compound.

2. The method of claim 1 wherein said peroxyl radical initiator comprises a 2,2′-azobis(2-amidinopropane) salt.

3. The method of claim 2 wherein said 2,2′-azobis(2-amidinopropane) salt comprises 2,2′-azobis(2-amidinopropane) dihydrochloride.

4. The method of claim 1, further comprising comparing said antioxidant capacity of said test compound to an antioxidant capacity of a standard compound, wherein said antioxidant capacity of a standard compound is generated by the steps of:
(a) contacting a cultured cell with 2′,7′-dichlorofluorescin diacetate in the presence of a standard compound, wherein the 2′,7′-dichlorofluorescin diacetate enters the cell and is cleaved to 2′,7′-dichlorofluorescin;
(b) contacting the cell with a peroxyl radical initiator;
(c) measuring fluorescence in an emission wavelength of 2′,7′dichlorofluorescein at a plurality of time points; and
(d) determining the area-under-the-curve of a graph plotting 2′,7′-dichlorofluorescein diacetate fluorescence vs. time for the cultured cell in the presence the standard compound.

5. The method of claim 4, wherein said standard compound is selected from the group consisting of quercetin, galangin, EGCG and kaempferol.

6. The method of claim 1 wherein said emission wavelength is 538 nm.

7. The method of claim 1 wherein said test compound is produced by a plant.

8. The method of claim 7 wherein said test compound is a phytochemical.

9. The method of claim 1 wherein said cultured cell is a eukaryotic cell.

10. The method of claim 9 wherein said eukaryotic cell is a human cell.

11. The method of claim 9 wherein said eukaryotic cell is a cell of a human cell line.

12. (canceled)

13. The method of claim 1, further comprising a step of washing said cultured cell prior to the step of contacting said cell with said peroxyl initiator and comparing antioxidant activity data derived from washed cells with antioxidant activity data derived from unwashed cells.

14. A method of predicting in vivo antioxidant capacity of a compound, the method comprising the steps of:
a) contacting a first cultured cell with 2′,7′-dichlorofluorescin diacetate, in the presence of a test compound to form a first mixture,
b) contacting a second cultured cell with 2′,7′-dichlorofluorescin diacetate, in the absence of said test compound to form a second mixture, wherein said 2′,7′-dichlorofluorescin diacetate enters said first and said second cells and is cleaved therein to 2′,7′-dichlorofluorescin;
c) contacting said first and second mixtures with a peroxyl radical initiator; and
d) measuring fluorescence in an emission wavelength of 2′,7′-dichlorofluorescein at a plurality of time points in said first and said second mixtures,
e) determining area-under-the-curve of a graph plotting 2′,7′-dichlorofluorescein diacetate fluorescence vs. time for said first and second mixtures,
wherein a decrease in said area-under-the-curve at an emission wavelength of 2′,7′-dichlorofluorescein in said first mixture, relative to said area-under-the-curve in said second mixture provides a prediction of in vivo antioxidant capacity of said test compound.

15. (canceled)

16. (canceled)

17. (canceled)

18. (canceled)

19. (canceled)

20. (canceled)

21. (canceled)

22. (canceled)

23. (canceled)

24. (canceled)

25. A kit for measuring the antioxidant capacity of a compound, the kit comprising:
a) 2′,7′-dichlorofluorescin diacetate;
b) a peroxyl radical initiator;
c) a standard;
d) computer readable medium comprising instructions for determining antioxidant capacity of a test compound, and
d) packaging materials therefor.

26. The kit of claim 25, further comprising a viable eukaryotic cell.

27. (canceled)

28. (canceled)

29. The kit of claim 25, wherein said peroxyl radical initiator comprises a 2,2′-azobis(2-amidinopropane) salt.

30. (canceled)

31. (canceled)

32. A method for determining an absolute value of antioxidant activity for a test compound, the method comprising the steps of:
a. contacting a first cultured cell with 2′,7′-dichlorofluorescin diacetate, in the presence of a test compound,
b. contacting a second cultured cell with 2′,7′-dichlorofluorescin diacetate, in the absence of said test compound, wherein said 2′,7′-dichlorofluorescin diacetate enters said first and second cells and is cleaved therein to 2′,7′-dichlorofluorescin;
d. contacting said first and second cells with a peroxyl radical initiator; and
e. measuring fluorescence in an emission wavelength of 2′,7′-dichlorofluorescein at a plurality of time points in said first and second cultured cells,
f. determining the ratio of area-under-the-curve of a graph plotting 2′,7′-dichlorofluorescein diacetate fluorescence vs. time for said first and second cultured cells,
g. normalizing the ratio of area-under-the-curve of step (f) to area-under-the-curve of a graph plotting 2′,7′-dichlorofluorescein diacetate fluorescence vs. time for a standard compound.

33. (canceled)

34. The method of claim 32, wherein an absolute value of antioxidant activity is determined for a test compound by applying the values obtained in claim 31 to Equation (1)
$$CAA abs = \frac{( 1 - ( \int SA / \int CA ) )}{( 1 - ( \int Sq / \int CA ) )} \qquad Equation ( 1 )$$
wherein ∫SA is the area-under-the-curve for fluorescence vs. time of said test compound, ∫CA is the area-under-the-curve for fluorescence vs. time in the absence of said test compound, and ∫S_{q} is the area-under-the-curve for fluorescence vs. time of said standard compound, and wherein CAA_{abs} is the absolute value of antioxidant activity for a test compound.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. A computer-readable medium comprising instructions for obtaining an absolute antioxidant value from fluorescence measured at a plurality of time points, the medium comprising:
(a) instructions for receiving a plurality of fluorescence values, the values representing fluorescence at a plurality of time points for a cultured cell in the presence and absence of a test compound;
(b) instructions for receiving a plurality of fluorescence values, the values representing fluorescence at a plurality of time points for a cultured cell in the presence of a standard compound;
(c) instructions for calculating an absolute antioxidant value, CAA_{abs}, for said test compound, said instructions comprising applying the values received according to instructions (a) and (b) to the relationship of Equation (1)
$$CAA abs = \frac{( 1 - ( \int SA / \int CA ) )}{( 1 - ( \int Sq / \int CA ) )} \qquad Equation ( 1 )$$
wherein ∫SA is the area-under-the-curve for fluorescence vs. time of said test compound, ∫CA is the area-under-the-curve for fluorescence vs. time in the absence of said test compound, and ∫S_{q} is the area-under-the-curve for fluorescence vs. time of said standard compound; and
(d) instructions for transmitting a value for CAA_{abs} to an output device.
